(19) United States
(12) Patent Application Publication
(10) Pub. No.: US 2026/0057139 A1
(43) Pub. Date: Feb. 26, 2026

(54) METHODS AND SYSTEMS FOR PHENOTYPIC FIT ANALYSIS

(71) Applicant: GeneDx, LLC, Gaithersburg, MD (US)

(72) Inventors: Vincent D. Ustach, Baltimore, MD (US); Vladimir G. Gainullin, Rockville, MD (US); Stephen McGee, Greenville, SC (US); Kevin Arvai, Alexandria, VA (US)

(21) Appl. No.: 19/182,385

(22) Filed: Apr. 17, 2025

Related U.S. Application Data
(60) Provisional application No. 63/686,202, filed on Aug. 23, 2024.
(60) Provisional application No. 63/743,768, filed on Jan. 10, 2025.

(51) Int. Cl. G06F 30/20; G16B 20/00; G16B 30/00; G16B 40/20

(52) U.S. Cl. CPC G06F 30/20; G16B 20/00; G16B 30/00; G16B 40/20

(57) ABSTRACT

The present disclosure provides a method for performing phenotypic fit analysis. The method comprises computer processing an input dataset to produce a set of genes and a set of gene-phenotype associations (GPAs) associated with the set of genes. The method further comprises determining, for a subject, a plurality of subject-gene similarity subscores, based at least in part on the GPAs associated with the set of genes. The method further comprises determining a predicted likelihood of association between the subject and at least a subset of the set of genes, based at least in part on the plurality of subject-gene similarity subscores.

### CROSS-REFERENCE

This application claims the benefit of U.S. Provisional Application No. 63/686,202, filed Aug. 23, 2024, and U.S. Provisional Application No. 63/743,768, filed Jan. 10, 2025, which are each incorporated herein by reference in their entirety.

### BACKGROUND

Diagnostic genetic testing may involve examining a subject's DNA to detect genetic mutations that may cause illness or disease. Diagnostic genetic testing may play a fundamental role in determining an individual's risk of developing certain diseases and uncovering genetic causes of illnesses that a subject may be suffering from.

### SUMMARY

The present disclosure recognizes a need for improved methods for analyzing relationships between a subject's clinical symptoms and their genetic profile.

In an aspect, the present disclosure provides a method for performing phenotypic fit analysis, comprising: (a) computer processing an input dataset to produce a set of genes and a set of gene-phenotype associations (GPAs) associated with the set of genes; (b) determining, for a subject, a plurality of subject-gene similarity subscores, based at least in part on the GPAs associated with the set of genes; and (c) determining a predicted likelihood of association between the subject and at least a subset of the set of genes, based at least in part on the plurality of subject-gene similarity subscores.

In some embodiments, the method further comprises performing automated curation and integration of a plurality of discrete datasets to generate the input dataset. In some embodiments, the input dataset comprises one or more of: disease-phenotype annotations, patient descriptions, and structured clinical data. In some embodiments, the method further comprises extracting the disease-phenotype annotations from one or more databases. In some embodiments, the method further comprises performing literature mining on a set of published literature to produce the patient descriptions. In some embodiments, the literature mining comprises automated retrieval and formatting of text documents to construct a comprehensive corpus. In some embodiments, the literature mining comprises processing the comprehensive corpus using natural language processing (NLP) to identify GPAs without manual discovery. In some embodiments, the literature mining comprises generating vector embeddings from the text documents in the comprehensive corpus using a trained doc2vec model.

In some embodiments, the method further comprises generating a dec2vec similarity subscore based at least in part on comparing the patient descriptions to the text documents in the comprehensive corpus. In some embodiments, the literature mining comprises training a doc2vec model configured to learn and generate vector embeddings from the text documents in the comprehensive corpus. In some embodiments, the patient descriptions comprise at least one unpublished gene that is not present in the OMIM-ORPHANET database.

In some embodiments, the method further comprises generating the structured clinical data. In some embodiments, the structured clinical data comprises electronic health records (EHR), or at least one unpublished gene not present in the OMIM-ORPHANET database, or both. In some embodiments, (a) comprises computer processing Human Phenotype Ontology (HPO) terms, or non-exact HPO terms, or both. In some embodiments, the GPAs are columnar data of phenotypes associated with each of the set of genes.

In some embodiments, the method further comprises collecting the GPAs for a given gene of the set of genes by grouping subjects having a causative variant on the given gene, and flattening the set of phenotypes for each subject. In some embodiments, (b) is based at least in part on a set of phenotypes of the subject. In some embodiments, the plurality of subject-gene similarity subscores comprises at least 2, at least 3, at least 4, at least 5, at least 6, at least 7, at least 8, or more than 8 different subject-gene similarity subscores. In some embodiments, the plurality of subject-gene similarity subscores comprises a hybrid relative semantic similarity (HRSS) score, a Jaccard similarity score, a word2vec cosine similarity score, a doc2vec score, or a combination thereof. In some embodiments, (b) is based at least in part on protein-protein interactions, gene family data, orthologs, paralogs, biological pathways, gene-phenotype data from animal sources, or a combination thereof. In some embodiments, the predicted likelihood of association comprises a predicted probability of association. In some embodiments, (c) comprises use of a trained machine learning model. In some embodiments, the trained machine learning model is selected from the group consisting of a random forest, a neural network, a decision tree, a k-nearest neighbor, a linear regression, a logistic regression, and a combination thereof. In some embodiments, the trained machine learning model is trained using case data to learn relationships between a diagnostic and non-diagnostic disease state of a gene in a subject case and the plurality of subject-gene similarity subscores. In some embodiments, the method is performed without genotype information of the subject. In some embodiments, the method further comprises prioritizing the at least the subset of the set of genes, based at least in part on the predicted likelihood of association determined in (d). In some embodiments, the prioritizing is based at least in part on a set of HPO terms assigned to the subject, medical text describing the subject, and the set of genes. In some embodiments, the input dataset comprises case data for a set of subjects.

In an aspect, the present disclosure provides a system for performing phenotypic fit analysis, comprising: a database that is configured to store an input dataset; and one or more computer processors operatively coupled to the database, wherein the one or more computer processors are individually or collectively programmed to: (i) process an input dataset to produce a set of genes and a set of gene-phenotype associations (GPAs) associated with the set of genes; (ii) determine, for a subject, a plurality of subject-gene similarity subscores, based at least in part on the GPAs associated with the set of genes; and (iii) determine a predicted likelihood of association between the subject and at least a subset of the set of genes, based at least in part on the plurality of subject-gene similarity subscores.

In an aspect, the present disclosure provides a non-transitory computer readable medium comprising machine-executable code that, upon execution by one or more computer processors, implements a method for performing phenotypic fit analysis, the method comprises: (a) computer processing an input dataset to produce a set of genes and a set of gene-phenotype associations (GPAs) associated with the set of genes; (b) determining, for a subject, a plurality of subject-gene similarity subscores, based at least in part on the GPAs associated with the set of genes; and (c) determining a predicted likelihood of association between the subject and at least a subset of the set of genes, based at least in part on the plurality of subject-gene similarity subscores.

In an aspect, the present disclosure provides a method for performing phenotypic fit analysis, comprising: (a) computer processing an input dataset to produce a set of genes and a set of gene-phenotype associations (GPAs) associated with the set of genes; (b) determining, for a subject, a plurality of subject-gene similarity subscores, based at least in part on the GPAs associated with the set of genes; and (c) determining a predicted likelihood of association between the subject and at least a subset of the set of genes, based at least in part on the plurality of subject-gene similarity subscores.

In some embodiments, the method further comprises performing automated curation and integration of a plurality of discrete datasets to generate the input dataset. In some embodiments, the input dataset comprises one or more of: disease-phenotype annotations, patient descriptions, and structured clinical data. In some embodiments, the input dataset comprises two or more of: disease-phenotype annotations, patient descriptions, and structured clinical data. In some embodiments, the input dataset comprises disease-phenotype annotations, patient descriptions, and structured clinical data. In some embodiments, the method further comprises extracting the disease-phenotype annotations from one or more databases. In some embodiments, the one or more databases comprise Online Mendelian Inheritance in Man (OMIM), Orphanet, GeneReviews, or a combination thereof.

In some embodiments, the method further comprises performing literature mining on a set of published literature to produce the patient descriptions. In some embodiments, the literature mining comprises automated retrieval and formatting of text documents to construct a comprehensive corpus. In some embodiments, the literature mining comprises processing the comprehensive corpus using natural language processing (NLP) to identify GPAs without manual discovery. In some embodiments, the text documents are retrieved from one or more article databases. In some embodiments, the article databases comprise MEDLINE/Pubmed, PMC articles from NCBI, GeneReviews, or a combination thereof. In some embodiments, the literature mining comprises generating vector embeddings from the text documents in the comprehensive corpus. In some embodiments, the vector embeddings are generated using a trained doc2vec model. In some embodiments, the method further comprises generating a dec2vec similarity subscore based at least in part on comparing the patient descriptions to the text documents in the comprehensive corpus. In some embodiments, the literature mining comprises training a doc2vec model configured to learn and generate vector embeddings from the text documents in the comprehensive corpus. In some embodiments, the patient descriptions comprise at least one gene that is not present in the OMIM-ORPHANET database. In some embodiments, the method further comprises generating the structured clinical data. In some embodiments, the structured clinical data comprises electronic health records (EHR). In some embodiments, the structured clinical data comprises at least one unpublished gene. In some embodiments, the at least one unpublished gene is not present in the OMIM-ORPHANET database.

In some embodiments, (a) comprises computer processing Human Phenotype Ontology (HPO) terms.

In some embodiments, (a) comprises computer processing non-exact HPO terms. In some embodiments, (a) comprises determining semantic similarity between HPO terms and/or non-exact HPO terms.

In some embodiments, the GPAs are columnar data of phenotypes associated with each of the set of genes. In some embodiments, the method further comprises collecting GPAs for a given gene of the set of genes by grouping subjects having a causative variant on the given gene, and flattening the set of phenotypes for each subject.

The method of claim 1, wherein (b) is further based at least in part on a set of phenotypes of the subject.

In some embodiments, the plurality of subject-gene similarity subscores comprises at least 2, at least 3, at least 4, at least 5, at least 6, at least 7, at least 8, or more than 8 different subject-gene similarity subscores.

In some embodiments, the plurality of subject-gene similarity subscores comprises a hybrid relative semantic similarity (HRSS) score, a Jaccard similarity score, a word2vec cosine similarity score, a doc2vec score, or a combination thereof.

In some embodiments, (b) is further based at least in part on protein-protein interactions, gene family data, orthologs, paralogs, biological pathways, gene-phenotype data from animal sources, or a combination thereof.

In some embodiments, the predicted likelihood of association comprises a predicted probability of association.

In some embodiments, (c) comprises use of a trained machine learning model. In some embodiments, the trained machine learning model is selected from the group consisting of a random forest, a neural network, a decision tree, a k-nearest neighbor, a linear regression, a logistic regression, and a combination thereof. In some embodiments, the trained machine learning model is trained using case data to learn relationships between a diagnostic or non-diagnostic disease state of a gene in a subject case and the plurality of subject-gene similarity subscores.

In some embodiments, the method is performed without genotype information of the subject.

In some embodiments, the method further comprises prioritizing the at least the subset of the set of genes, based at least in part on the predicted likelihood of association determined in (d). In some embodiments, the prioritizing is based at least in part on a set of HPO terms assigned to the subject, medical text describing the subject, and the set of genes.

In some embodiments, the input dataset comprises case data for a set of subjects.

In another aspect, the present disclosure provides a system for performing phenotypic fit analysis, comprising: a database that is configured to store an input dataset; and one or more computer processors operatively coupled to the database, wherein the one or more computer processors are individually or collectively programmed to: (i) process an input dataset to produce a set of genes and a set of gene-phenotype associations (GPAs) associated with the set of genes; (ii) determine, for a subject, a plurality of subject-gene similarity subscores, based at least in part on the GPAs associated with the set of genes; and (iii) determine a predicted likelihood of association between the subject and at least a subset of the set of genes, based at least in part on the plurality of subject-gene similarity subscores.

In another aspect, the present disclosure provides a non-transitory computer readable medium comprising machine-executable code that, upon execution by one or more computer processors, implements a method for performing phenotypic fit analysis, the method comprises: (a) computer processing an input dataset to produce a set of genes and a set of gene-phenotype associations (GPAs) associated with the set of genes; (b) determining, for a subject, a plurality of subject-gene similarity subscores, based at least in part on the GPAs associated with the set of genes; and (c) determining a predicted likelihood of association between the subject and at least a subset of the set of genes, based at least in part on the plurality of subject-gene similarity subscores.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows how retrospective internal SDP case data was split into gene-phenotype annotations, training data, and proof-of-concept test data.

FIG. 2 shows how the gene prioritization tool outperformed the prioritization from individual subscores over the test set.

FIG. 3 shows how as the internal positive case count of the diagnostic gene increased, the predictive ability increased.

FIG. 4 shows that the performance of the tool was independent of the number of HPO terms.

FIG. 5 shows how the term frequency-inverse document frequency (TF-IDF) of all the terms in each gene group were evaluated.

FIG. 6 displays a tally, for each subscore, of the number of cases for which that score had the highest rank for the diagnostic gene, separated by SDC1 case count of the diagnostic gene.

FIG. 7 shows the number of PMIDs every year between 1950 and 2020.

FIG. 8 displays mentions of unique HPO terms and gene symbols between 1951 and 2020.

FIG. 9 shows a computer system that is programmed or otherwise configured to implement methods provided herein.

FIGS. 10A-10F show evaluation of absolute score (Multiscore value) versus rank of diagnostic gene. FIG. 10A shows a histogram of fractional rank of diagnostic gene amongst all case genes. A rank of 0.0 means the diagnostic gene had the highest Multiscore value of all case genes (the most desirable result), and a rank of 1.0 means the diagnostic gene had the lowest Multiscore value. Height of bar is the number of cases with the given rank. FIG. 10B shows a histogram of diagnostic gene rank (blue) overlaid with histogram of diagnostic gene Multiscore value (orange) shown as distance or 1-Multiscore value to easily compare the different metrics. Vertical axis is shown as log scale of case count to elucidate the differences on the long tail of poor performance (high rank, low score). FIG. 10C shows a scatter plot of Multiscore value versus rank of diagnostic gene. Each point is a single case. Green dotted line represents y=x. FIG. 10D shows a scatter plot of mean IC of all case HPO terms versus median Multiscore value of all case. Each point is a single case. Line of best fit shown in black with R2-0.04. FIG. 10E shows a histogram of Multiscore values of all case genes depicting a case with many high scoring case genes. The diagnostic gene Multiscore value (cat1 score) is 0.971 which is the top ranked value (cat1 frac=0.0). FIG. 10F shows a histogram of Multiscore values of all case genes depicting a case with low scoring case genes. The diagnostic gene Multiscore value (cat1 score) is 0.266, however this is still the top ranked value (cat1 frac=0.0).

FIGS. 11A-11C show gene cohort sizes for 2,350 unique genes across 26,586 Single Diagnosis Category 1 (SDC1) cases in DiPR. FIG. 11A shows a histogram of number of SDC1 DiPR cases per gene. Height of bar is the number of gene cohorts with the given bin size, represented on a log scale. Bin size=10 cases. FIG. 11B shows a bar chart of gene counts per cohort size, grouped into singleton (1 case), small cohort (2-5 cases), medium cohort (6-20), and large cohort (more than 21 cases). FIG. 11C shows the number of cases represented by the genes in the cohort sizes in FIG. 11B. These data indicate that most of the 2,350 genes with SDC1 cases are members of singleton or small gene cohorts, however, most of the SDC1 cases are members of large gene cohorts.

FIG. 12 shows case performance separated into gene cohort size of the diagnostic gene. In the analysis, gene cohort sizes were assigned per case and were determined by SDC1 cases that were closed before the month of the case being tested (see main text for details). Diagnostic genes tended to have higher score ranks (worse performance) when there was a low number of SDC1 cases in the DiPR data table. The histograms have been normalized such that the area underneath the histogram is equal for all five cohort sizes.

FIG. 13 shows case performance separated into genes with single diseases and genes with multiple diseases. Disease count was taken from GeneMb validated gene diseases. Blue: Genes with a single disease. Orange: genes with multiple diseases. Genes with multiple diseases were found to have a higher proportion of cases with rank less than 0.02. Vertical axis is visualized with log scale.

FIG. 14A shows an overview of the genotype and phenotype analysis schema. Genotype and phenotype analyses are handled independently. Genotype is used for filtering (genotype-first approach) and phenotype informs the final selection of variants for reporting during clinical analysis.

FIG. 14B shows an example of how the schema works for genes not yet curated in public gene-phenotype association databases. HPOA: disease-phenotype annotations extracted from OMIM using the Sep. 1, 2023 release.

FIG. 15 shows analysis of subscores within Multiscore. Top: Stacked bar plot representation of the tally of the number of cases for which that score had the highest rank for the diagnostic gene. Blue=outright wins, orange=tied for highest. Bottom: Dᵣₐₙₖ; median difference between the score and the winner, for cases where the score was not the winner.

FIG. 16 shows splitting retrospective training case set into gene-phenotype annotations, training data, and testing data. The vertical axis reflects the date of cases closed, which acts as a filter and grouping mechanism for case data. For each month of cases, separated by the date clinical genetic testing was completed, the eight subscores were generated based on the internal and external knowledge available on the last day of the previous month.

FIG. 17 shows an average recall at k of the reported positive finding gene for Multiscore and the subscore features.

FIG. 18 shows an average recall at k of the reported positive finding gene for Multiscore and the benchmarked tools.

FIG. 19 shows an analysis of benchmark analysis comparing Multiscore to Phrank using GDx GPAs, Phrank using HPOA GPAs, and LIRICAL using HPOA and ORPHANET GPAs. Top: Stacked bar plot representation of the tally of the number of cases for which that score had the highest rank for the diagnostic gene. Blue=outright wins, orange=tied for highest. Bottom: Dᵣₐₙₖ; median difference between the score and the winner, for cases where the score was not the winner.

FIG. 20 shows a scatter plot of number of HPO terms assigned to a patient case (horizontal axis) versus the rank of the positive gene (vertical axis).

FIG. 21 shows histogram plots of the number of genes with a given median TF-iDF (horizontal axis) for all internal (GDx) gene-phenotype associations. The histograms are separated by bins of case count.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out.

Diagnostic genomics testing may rely on a subject's (e.g., patient's) genotype to inform filtering strategies and may use their phenotype to ultimately inform reporting. Determining the correlation between clinical features and disease-causing genes may require advanced clinical expertise and extensive knowledge about the physical manifestations of gene variation. The present disclosure recognizes a need for improved methods for analyzing relationships between a subject's clinical symptoms and their genetic profile. The methods and systems described herein may combine curated datasets of gene-phenotype associations and real-world data to rank the likelihood of a match between a gene and an individual's phenotype, with high precision and independently of genotype. Incorporating real patient data may remove the bias against individuals with milder disease or disorders with variable expressivity that may be easily missed by other tools, which may expect perfect matches with the initial cohorts of selected individuals reported in the literature.

In an aspect, the present disclosure provides a method for performing phenotypic fit analysis, comprising: (a) computer processing an input dataset to produce a set of genes and a set of gene-phenotype associations (GPAs) associated with the set of genes; (b) determining, for a subject, a plurality of subject-gene similarity subscores, based at least in part on the GPAs associated with the set of genes; and (c) determining a predicted likelihood of association between the subject and at least a subset of the set of genes, based at least in part on the plurality of subject-gene similarity subscores.

In some embodiments, the input dataset may comprise one or more of: disease-phenotype annotations, patient descriptions, and structured clinical data. In some embodiments, the input dataset may comprise two or more of: disease-phenotype annotations, patient descriptions, and structured clinical data. In some embodiments, the input dataset may comprise disease-phenotype annotations, patient descriptions, and structured clinical data. In some embodiments, the one or more databases may comprise Online Mendelian Inheritance in Man (OMIM), Orphanet, GeneReviews, or a combination thereof. In some embodiments, the literature mining may comprise automated retrieval and formatting of text documents to construct a comprehensive corpus. In some embodiments, the literature mining may comprise processing the comprehensive corpus using natural language processing (NLP) to identify GPAs without manual discovery. In some embodiments, the text documents may be retrieved from one or more article databases. In some embodiments, the article databases may comprise MEDLINE/Pubmed, PMC articles from NCBI, GeneReviews, or a combination thereof. In some embodiments, the literature mining may comprise generating vector embeddings from the text documents in the comprehensive corpus. In some embodiments, the vector embeddings may be generated using a trained doc2vec model. In some embodiments, the literature mining may comprise training a doc2vec model configured to learn and generate vector embeddings from the text documents in the comprehensive corpus. In some embodiments, the patient descriptions may comprise at least one gene that is not present in the OMIM-ORPHANET database. In some embodiments, the structured clinical data may comprise electronic health records (EHR). In some embodiments, the structured clinical data may comprise at least one unpublished gene. In some embodiments, the at least one unpublished gene may not be present in the OMIM-ORPHANET database. In some embodiments, (a) may comprise computer processing Human Phenotype Ontology (HPO) terms. In some embodiments, (a) may comprise computer processing non-exact HPO terms. In some embodiments, (a) may comprise determining semantic similarity between HPO terms and/or non-exact HPO terms. In some embodiments, the GPAs may be columnar data of phenotypes associated with each of the set of genes. In some embodiments, (b) may be further based at least in part on a set of phenotypes of the subject. In some embodiments, the plurality of subject-gene similarity subscores may comprise at least 2, at least 3, at least 4, at least 5, at least 6, at least 7, at least 8, or more than 8 different subject-gene similarity subscores. In some embodiments, the plurality of subject-gene similarity subscores may comprise a hybrid relative semantic similarity (HRSS) score, a Jaccard similarity score, a word2vec cosine similarity score, a doc2vec score, or a combination thereof. In some embodiments, (b) may be further based at least in part on protein-protein interactions, gene family data, orthologs, paralogs, biological pathways, gene-phenotype data from animal sources, or a combination thereof. In some embodiments, the predicted likelihood of association may comprise a predicted probability of association. In some embodiments, (c) may comprise use of a trained machine learning model. In some embodiments, the trained machine learning model may be selected from the group consisting of a random forest, a neural network, a decision tree, a k-nearest neighbor, a linear regression, a logistic regression, and a combination thereof. In some embodiments, the method may be performed without genotype information of the subject. In some embodiments, the prioritizing may be based at least in part on a set of HPO terms assigned to the subject, medical text describing the subject, and the set of genes.

In another aspect, the present disclosure provides a system for performing phenotypic fit analysis. The system may comprise a database that is configured to store an input dataset; and one or more computer processors operatively coupled to the database, wherein the one or more computer processors are individually or collectively programmed to: (i) process an input dataset to produce a set of genes and a set of gene-phenotype associations (GPAs) associated with the set of genes; (ii) determine, for a subject, a plurality of subject-gene similarity subscores, based at least in part on the GPAs associated with the set of genes; and (iii) determine a predicted likelihood of association between the subject and at least a subset of the set of genes, based at least in part on the plurality of subject-gene similarity subscores.

In another aspect, the present disclosure provides a non-transitory computer readable medium comprising machine-executable code that, upon execution by one or more computer processors, implements a method for performing phenotypic fit analysis. The method may comprise: (a) computer processing an input dataset to produce a set of genes and a set of gene-phenotype associations (GPAs) associated with the set of genes; (b) determining, for a subject, a plurality of subject-gene similarity subscores, based at least in part on the GPAs associated with the set of genes; and (c) determining a predicted likelihood of association between the subject and at least a subset of the set of genes, based at least in part on the plurality of subject-gene similarity subscores.

### Computer Systems

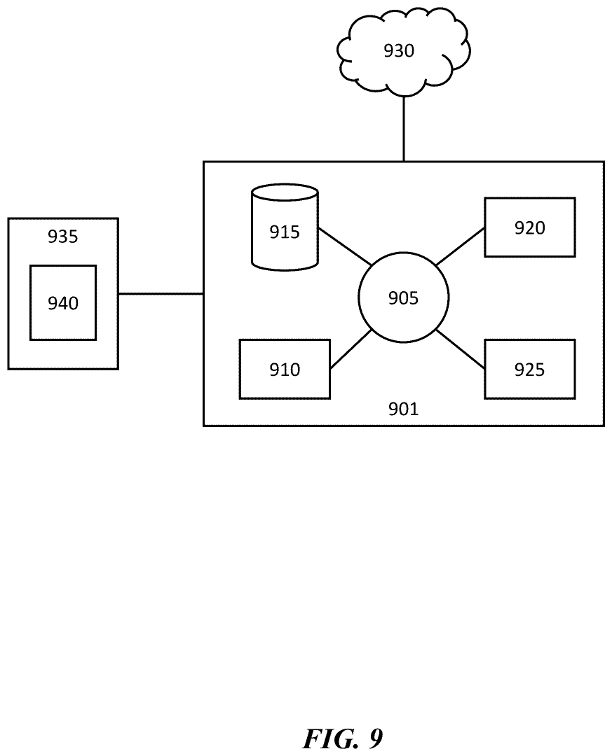

The present disclosure provides computer systems that are programmed to implement methods of the disclosure. FIG. 9 shows a computer system 901 that is programmed or otherwise configured to predict phenotypes from genes. The computer system 901 can regulate various aspects of the present disclosure, such as, for example, gene prioritization or surveying literature. The computer system 901 can be an electronic device of a user or a computer system that is remotely located with respect to the electronic device. The electronic device can be a mobile electronic device.

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet. The network 930 in some cases is a telecommunication and/or data network. The network 930 can include one or more computer servers, which can enable distributed computing, such as cloud computing. The network 930, in some cases with the aid of the computer system 901, can implement a peer-to-peer network, which may enable devices coupled to the computer system 901 to behave as a client or a server.

The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 901 can be included in the circuit. In some cases, the circuit is an application specific integrated circuit (ASIC).

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 901 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 901 via the network 930.

Methods as described herein can be implemented by way of machine (e.g., computer processor) executable code stored on an electronic storage location of the computer system 901, such as, for example, on the memory 910 or electronic storage unit 915. The machine executable or machine readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 935 that comprises a user interface (UI) 940 for providing, for example, information about gene prioritization. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 905. The algorithm can, for example, identify genes most likely to be responsible for a clinical presentation.

### EXAMPLES

### Example 1: Methods and Systems for Gene-Level Prediction of Phenotypic Fit

Diagnostic genomics testing may rely on a subject's (e.g., patient's) genotype to inform filtering strategies and may use their phenotype to ultimately inform reporting. Determining the correlation between clinical features and disease-causing genes may require advanced clinical expertise and extensive knowledge about the physical manifestations of gene variation. Attempts to aggregate and curate phenotype information led to the development of the Human Phenotype Ontology (HPO) (Robinson P N, Köhler S, Bauer S, Seelow D, Horn D, Mundlos S. The Human Phenotype Ontology: a tool for annotating and analyzing human hereditary disease. Am J Hum Genet. 2008 November; 83(5):610-5, which is incorporated by reference herein in its entirety), which established a standard vocabulary to describe phenotypes and multiple databases of gene-phenotype associations like the Online Mendelian Inheritance in Man (OMIM) (Amberger J, Bocchini C A, Scott A F, Hamosh A. McKusick's Online Mendelian Inheritance in Man (OMIM). Nucleic Acids Res. 2009 January; 37 (Database issue): D793-6, which is incorporated by reference herein in its entirety), Orphanet (Aymé S. Orphanet, un serveur d′informations sur les maladies rares [Orphanet, an information site on rare diseases]. Soins. 2003 January-February; (672):46-7. French, which is incorporated by reference herein in its entirety), and GeneReviews (Adam M P, Feldman J, Mirzaa G M, et al., editors. GeneReviews® Seattle (WA): University of Washington, Seattle; 1993-2024. ncbi.nlm.nih.gov/books/NBK1116/, which is incorporated by reference herein in its entirety).

Automated or semi-automated tools to assess phenotypic fit may be developed. These phenotype-based gene prioritization tools may compare the knowledge of a patient case to knowledge of a gene and may rely on curated datasets to prioritize genes or variants using similarity metrics, statistical models, or trained machine learning models. Some tools may use only the case phenotypes but supplement the gene-phenotype knowledge with protein-protein interactions, gene family data, orthologs, paralogs, biological pathways, and/or gene-phenotype data from animal sources. Other tools may combine multiple data types in a knowledge graph, use a classification algorithm, and/or use a graph neural network. Other tools may prioritize variants detected in a patient case and consider the phenotype similarity along with variant frequency and other predictors of pathogenicity. Some tools like AMELIE (Birgmeier J, Haeussler M, Deisseroth C A, et al. AMELIE speeds Mendelian diagnosis by matching patient phenotype and genotype to primary literature. Sci Transl Med. 2020, which is incorporated by reference herein in its entirety) may incorporate a literature search to construct associations between genes, variants, and phenotypes. This may address a major limitation of other models, although at the potential expense of deprioritizing unpublished variants.

However, tools may not rely solely on case phenotypes and gene phenotypes. Electronic health records (EHR) may be used to derive phenotype data. In this example, methods and systems were used to develop a gene prioritization tool using a machine learning model. This tool was able to combine multiple measures of case-gene phenotypic similarity informed by a large internal dataset of well-characterized individuals, in addition to external data sources, in order to provide gene-level predictions of phenotypic fit.

The methods and systems described herein combined curated datasets of gene-phenotype associations and real-world data to rank the likelihood of a match between a gene and an individual's phenotype, with high precision and independently of genotype. Incorporating real patient data removed the bias against individuals with milder disease or disorders with variable expressivity that may be easily missed by other tools that expect perfect matches with the initial cohorts of selected individuals reported in the literature.

### Methods:

Three main sources of data were used: disease-phenotype annotations extracted from Online Mendelian Inheritance in Man (OMIM), Orphanet, or GeneReviews (Human Phenotype Ontology Annotation (HPOA) dataset); patient descriptions mined from the relevant published literature by a literature surveyor tool (see Example 2); and an internal comprehensive structured dataset of clinical data. The structured dataset of clinical data described probands suspected of rare disease and referred for clinical exome sequencing. These individuals were diagnosed with positive findings of a Mendelian disease.

All three data sources were processed to produce gene-phenotype annotations (GPAs) associated with each gene, which were columnar data of all phenotypes associated with each gene. The GPAs used were collected from the internal dataset from patients diagnosed with positive findings of a Mendelian disease. The GPAs for a single gene were collected by grouping all patients diagnosed with a causative mutation on a given gene and flattening the full list of phenotypes for each patient. The cases used were single-gene diagnoses only.

Literature dataset GPAs were collected from phenotypes extracted from publications. The publications were associated with genes via the gene2pubmed database. HPOA dataset GPAs were collected from the disease-level OMIM-ORPHANET phenotype annotations. The HPOA dataset GPAs were flattened onto genes using the mim2gene table. The scoring also depended on a word2vec model and doc2vec model trained internally.

The tool considered the phenotypic profile of the patient and the list of genes. For each gene, data inputs and algorithms were combined to generate eight patient-gene similarity subscores. These subscores were then fed to a random forest (RF) model trained to predict the probability of association between the patient and the gene. To prioritize the genes in a patient case, the tool used a list of phenotype codes (HPO terms) assigned to the patient, medical text describing the patient (such as primary indication, past medical history, and differential diagnosis) and the list of genes to analyze. The literature surveyor tool generated snapshot models that included the publication date to enable fair training and testing of the tool in time-series experiments.

Seven subscores were based on three algorithms that manipulated the GPA datasets. The GPAs from the GDx, Lit, and HPOA datasets fed the hybrid relative semantic similarity (HRSS), Jaccard similarity, and word2vec cosine similarity reference algorithms. The eighth and final subscore was the doc2vec subscore, which did not use GPAs but rather embedded patient medical text as vector embeddings and compared these patient descriptions to sentences from publications in the literature surveyor tool corpus (Table 2).

A goal of the tool was to predict whether a variant on a given gene may deliver a diagnostic result based on phenotypic information alone. The eight subscores served as features for the machine learning classifier, which yielded a probability of disease association between a patient case and a list of genes. The classifier did not have knowledge of phenotypes or Mendelian disease. Rather, it learned the relationship between the combinations of feature subscores that were paired with positive or negative case/gene labels.

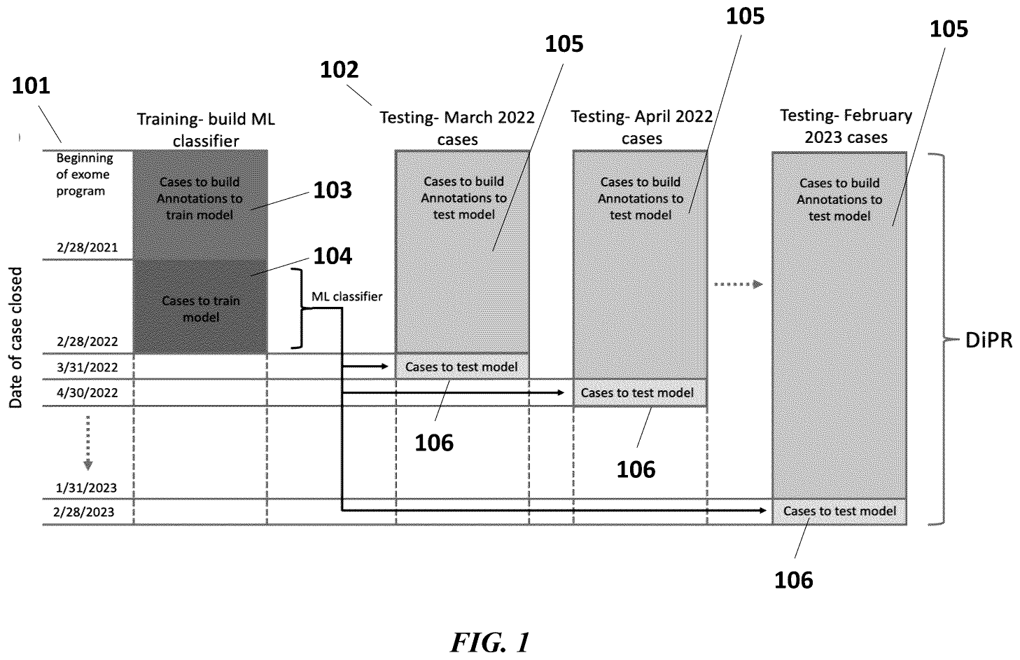

The classifier was trained on a set of positive cases with a single diagnostic gene finding analyzed by exome or genome sequencing internally. The training was devised as a time-series experiment and the internal, HPOA, and literature surveyor annotation data were collected to represent the internal and external knowledge available on the day before the training window (FIG. 1).

The cases were retrospectively tested in one-month batches based on the date the cases were closed (FIG. 1). The internal and literature surveyor gene-phenotype annotations and the literature surveyor doc2vec model were updated for each month batch. A word2vec model was used for all batches. The HPOA release from Sep. 1, 2023 was used for all batches.

In training the machine learning classifier 101, gene-phenotype associations were built from single-diagnosis positive (SDP) cases 103. These gene-phenotype associations had hybrid relative semantic similarity (HRSS), Jaccard similarity, and word2vec cosine similarity subscores. These cases were used to build the annotations for the model. Cases were used to train the model 104. All case genes (genes containing diagnostic and non-diagnostic pipeline variants) in these cases were scored to create the training data features and labels. The classifier model built during training makes predictions to give a final output value during testing and production. Also shown is testing of the machine learning classifier in a time-series proof-of-concept analysis 102. Cases were tested in monthly batches by date of case close. GPAs were built from SDP cases closed before the current test month 105. Cases closed during the current test month 106 are also shown.

### Results:

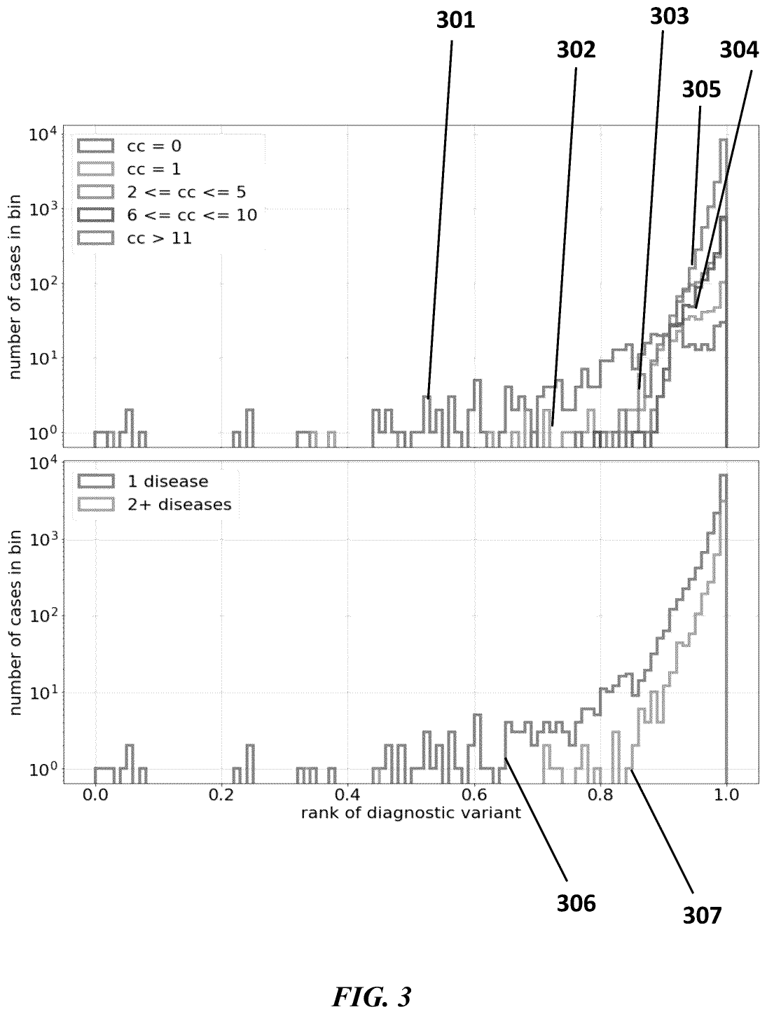

The gene prioritization tool was able to consistently prioritize diagnostic genes in retrospectively analyzed cases. Cases with a diagnostic finding in a single gene identified internally were used, excluding those used for the training dataset, to test model performances. The gene prioritization tool was able to prioritize the diagnostic gene in the top 9.8% of genes tested in 98% of cases (false negative rate of 2%), with a cohort size of 16,882 cases (Table 1). All genes associated with at least one mRNA transcript in the internal knowledge base were tested for each case. Case phenotypes were compared against gene phenotypes in the internal knowledge reference for 19,215 genes and translated to percentiles, with 100% being the top-ranked gene (e.g., best phenotypic fit). As the internal positive case count of the diagnostic gene increased, the predictive ability increased, allowing for stricter filtering thresholds at a constant false negative rate (Table 1 and FIG. 3).

TABLE 1
Filtering thresholds for allowed False Negative (FN) rates. The gene prioritization
tool prioritized the diagnostic gene in the top 9.8% of genes tested
in 98% of cases (FN rate of 2%). cc = diagnostic gene positive case
count seen internally before the present case was analyzed.

| FN rate | cc = 0 | cc = 1 | 2 <= cc <= 5 | 6 <= cc <= 10 | cc > 11 | all cc values |
|---|---|---|---|---|---|---|
| 1 | 0.061 | 0.685 | 0.882 | 0.911 | 0.934 | 0.845 |
| 2 | 0.288 | 0.746 | 0.897 | 0.917 | 0.945 | 0.902 |
| 5 | 0.525 | 0.856 | 0.917 | 0.931 | 0.960 | 0.935 |
| 10 | 0.660 | 0.899 | 0.931 | 0.947 | 0.971 | 0.957 |
| 25 | 0.818 | 0.931 | 0.959 | 0.971 | 0.985 | 0.980 |
| 50 | 0.897 | 0.963 | 0.984 | 0.990 | 0.994 | 0.993 |
| num cases | 425 | 446 | 1691 | 1539 | 12781 | 16882 |

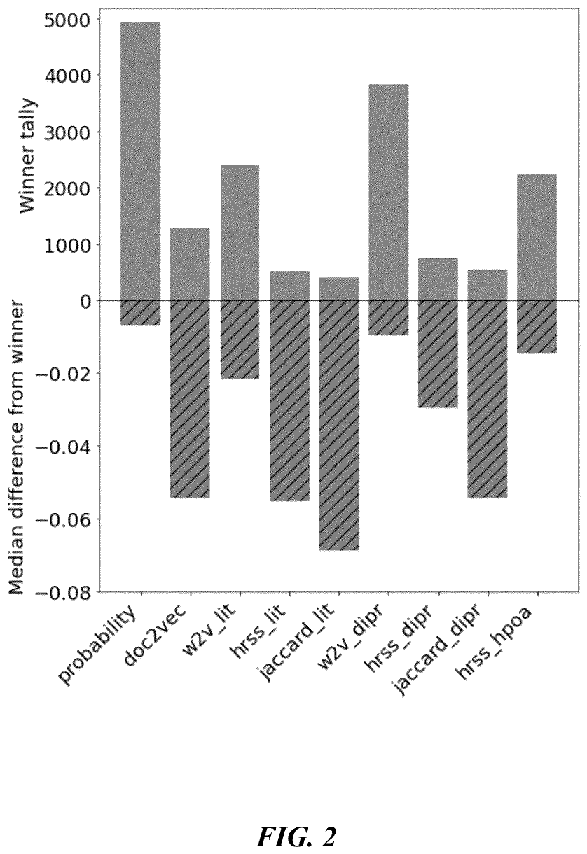

The gene prioritization tool outperformed the prioritization from individual subscores over the test set. The tally for each score, where a given score had the highest rank for the diagnostic gene, is shown in the top bar chart (solid bars) of FIG. 2. The gene prioritization tool RF probability was the highest-ranking score in 4,934 out of 16,876 (29.2%) cases tested. The w2v_dipr score was the highest-ranking score in 3,825 (22.7%) cases. For cases where a score was not the top rank, Dᵣₐₙₖ, the difference between that score's rank and the winning score's rank, was tracked. The bottom bar chart (diagonal lined bars) of FIG. 2 shows the median Dᵣₐₙₖ for each score. Median Dᵣₐₙₖ was lowest for RF probability with a value of −0.71%.

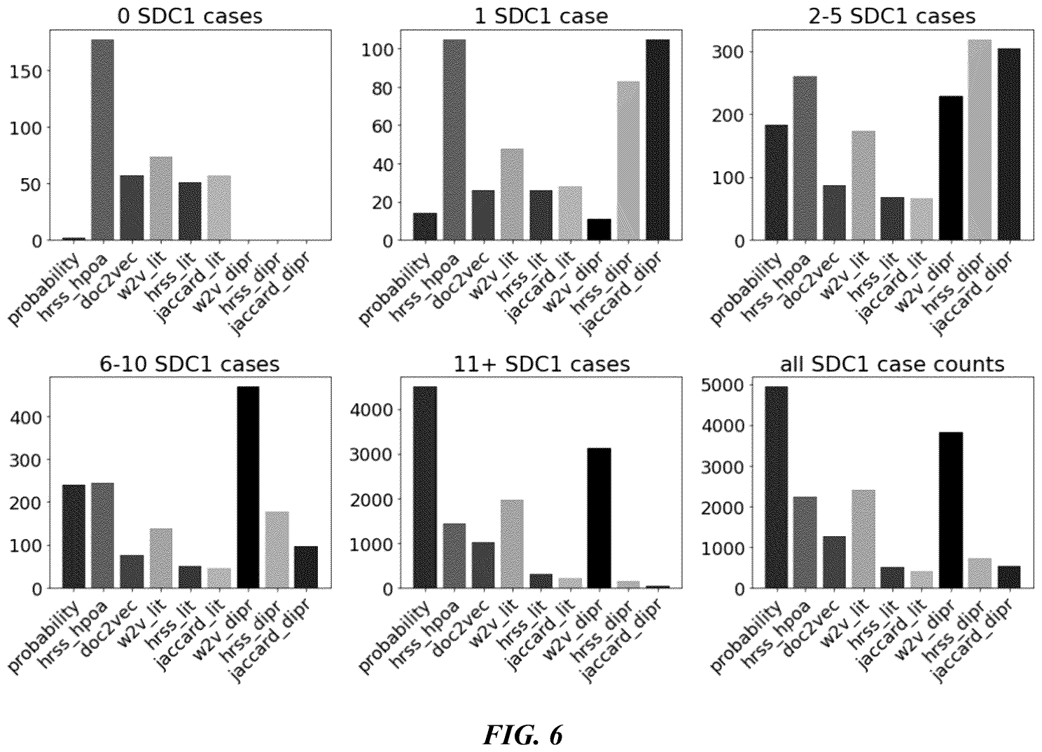

FIG. 6 displays a tally, for each subscore, of the number of cases for which that score had the highest rank for the diagnostic gene, separated by SDC1 case count of the diagnostic gene.

Phenotypic similarity assessed using HPOA or literature data individually resulted in a wider distribution of ranks, with 30-40% of diagnostic genes below the ninetieth percentile phenotypic rank. The tool also had a significant drop in performance when the diagnostic gene had not been previously reported in the internal dataset (Table 1).

Tool performance was not impacted when multiple diseases were associated with a gene. Some disease entries in the internal knowledge reference represented more than one disease in OMIM. Diseases were lumped following the guidance suggested by Thaxton et al. (PMID 35754516, which is incorporated by reference herein in its entirety). Disorders that shared a common inheritance pattern, mechanism of disease, and phenotypic features within and/or between families segregating the same variant(s) were reviewed by curators and lumped. These lumped disease entries represented a continuous spectrum of diseases, and based on case information, it was not always possible to say where in the spectrum a specific patient fell. For example, the CEP290 gene was associated with five diseases in OMIM: Bardet-Biedl syndrome 14 (MIM 615991), Joubert syndrome 5 (MIM 610188), Leber congenital amaurosis 10 (MIM 611755), Meckel syndrome 4 (MIM 611134), and Senior-Loken syndrome 6 (MIM 610189). These diseases present within an age range that spans prenatally all the way through adulthood, with overlapping clinical features. Thus, they were lumped under the CEP290-related ciliopathy umbrella. From a laboratory standpoint, when a patient is evaluated for this disorder, only an overall clinical fit was evaluated, rather than defining a more specific match.

However, when gene-level annotations are used, there may be concerns that the phenotypic information from several distinct diseases may be combined and affect scoring. The tool's performance on genes with a single disease association and on genes with multiple diseases were evaluated (FIG. 3). Diagnostic variants on genes with a single validated disease accounted for 12,385 cases. Diagnostic variants on genes with multiple validated diseases accounted for 4,495 cases. Linear regression analysis indicated a statistically significant positive relationship between number of diseases and rank. However, qualitatively, the distribution of ranks of these two classes of gene were similar. It was concluded that there was no major concern related to scoring genes with more than one disease.

TABLE 2
Description of data source and algorithm of each subscore.

| | | Scoring | |
|---|---|---|---|
| Scorer Name | Data Source | Algorithm | Note |
| hrss_hpoa | HPOA-OMIM | HRSS | Case HPO terms are compared |
| | annotation | | to all gene* HPO terms in |
| | file | | HPOA-OMIM |
| hrss_internal | Internal | HRSS | Case HPO terms are compared |
| | annotation file | | to all gene HPO terms from |
| | | | positive cases with a single |
| | | | diagnostic gene in the |
| | | | internal dataset |
| hrss_lit | Surveyor literature | HRSS | Case HPO terms are compared |
| | annotation file | | to all gene HPO terms found |
| | | | in literature mining |
| jaccard_internal | Internal | Jaccard | Case HPO terms are compared |
| | annotation file | similarity | to all gene HPO terms from |
| | | | positive cases with a single |
| | | | diagnostic gene in the |
| | | | internal dataset |
| jaccard_lit | Surveyor literature | Jaccard | Case HPO terms are compared |
| | annotation file | similarity | to all gene HPO terms found |
| | | | in literature mining |
| w2v_internal | Internal annotation file, | word2vec | Case HPO terms are compared |
| | word2vec model file | | to all gene HPO terms from |
| | | | positive cases with a single |
| | | | diagnostic gene in the |
| | | | internal dataset using HPO |
| | | | word embedding |
| w2v_lit | Surveyor literature | word2vec | Case HPO terms are compared |
| | annotation file, | | to all gene HPO terms found in |
| | word2vec model file | | literature mining using HPO |
| | | | word embedding |
| doc2vec | Surveyor doc2vec | doc2vec | Embedded case text is compared |
| | model file | | to embeddings of all documents; |
| | | | top score for each gene is the |
| | | | doc2vec score |

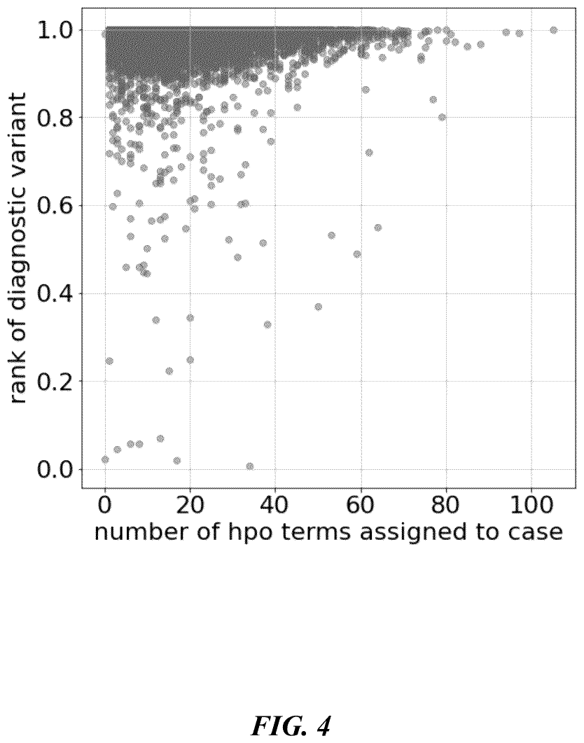

In contrast with other models, the tool was able to use patient data abstracted from clinical notes. The tool was also able to handle non-exact Human Phenotype Ontology (HPO) term matches. By using a combination of similarity scores, the tool recognized semantic similarity between two HPO terms by distance in the HPO hierarchy tree and information content. The tool also compared the overlap between the case and the gene knowledge dataset as a whole. The median number of HPO terms per case in the testing set was 14. The performance of the tool was independent of the number of HPO terms (FIG. 4). FIG. 4 displays a scatter plot of the number of HPOI terms assigned to a patient case (horizontal axis) versus the rank of the diagnostic variant (vertical axis).

The clinical data, which comprised the internal structured clinical dataset and the literature dataset, included 130 genes without OMIM or Orphanet entries that were able to be prioritized as well. This contributed to the diagnosis of 457 cases with diagnostic genes seen 6 times or more associated with newer and ultra-rare disorders that did not have OMIM or Orphanet entries.

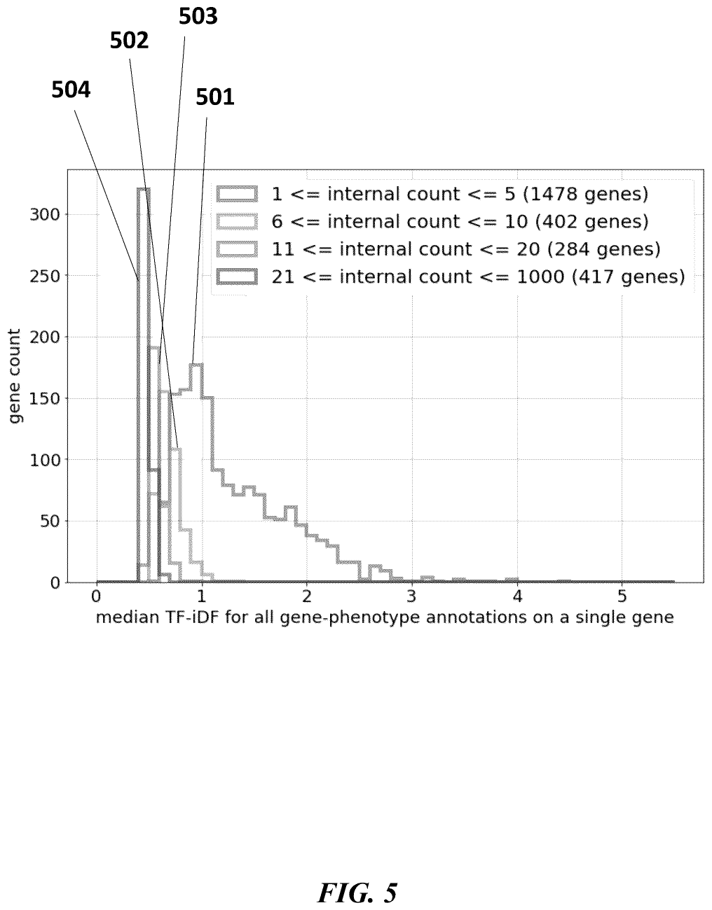

A relevant concern for using real world patient data instead of curated datasets is the introduction of “noise”, e.g., phenotypes present in the affected individual but unrelated to the diagnostic gene. Performance of the scores using the internal dataset was evaluated by the number of individuals included to create the gene reference information (Table 3). The term frequency-inverse document frequency (TF-IDF) of all the terms in each gene group were also evaluated (FIG. 5). TF-IDF may combine the frequency of a term within the cases for one gene with the presence of the term within all genes in the GPA corpus, and is a measure for term importance. As more cases are added to a gene group, the median GPA TF-IDF may decrease due to this noise. Performance improvements of the tool for genes that include >10 individuals in the knowledge reference increased only marginally and at the expense of incorporating low TF-IDF terms (FIG. 5, Table 3). FIG. 5 shows histogram plots of the median TF-iDF for all internal gene-phenotype associations. The histograms are separated by bins of case count. An internal count of 1-5 is indicated by 501. An internal count of 6-10 is indicated by 502. An internal count of 11-20 is indicated by 503. An internal count of 21-1000 is indicated by 504.

TABLE 3
Filtering thresholds and associated FN rates for high case count gene cases.

| FN rate | cc > 11 | cc > 15 | cc > 20 | cc > 25 | cc > 30 | cc > 30 | all cc values |
|---|---|---|---|---|---|---|---|
| 1 | 0.934 | 0.938 | 0.941 | 0.942 | 0.943 | 0.944 | 0.845 |
| 2 | 0.945 | 0.948 | 0.950 | 0.951 | 0.952 | 0.953 | 0.902 |
| 5 | 0.960 | 0.962 | 0.964 | 0.965 | 0.966 | 0.967 | 0.935 |
| 10 | 0.971 | 0.973 | 0.974 | 0.974 | 0.975 | 0.976 | 0.957 |
| 25 | 0.985 | 0.986 | 0.986 | 0.987 | 0.987 | 0.988 | 0.980 |
| 50 | 0.994 | 0.995 | 0.995 | 0.995 | 0.995 | 0.995 | 0.993 |
| num cases | 12781 | 11809 | 10782 | 9898 | 9054 | 8353 | 16882 |

Table 4 shows filtering thresholds and associated FN rates for genes with no HPOA data.

TABLE 4
Filtering thresholds and associated FN rates for genes with no HPOA data.

| FN rate | cc = 0 | cc = 1 | 2 <= cc <= 5 | 6 <= cc <= 10 | cc > 11 | all cc values |
|---|---|---|---|---|---|---|
| 1 | 0.028 | 0.359 | 0.781 | 0.900 | 0.908 | 0.157 |
| 2 | 0.046 | 0.400 | 0.886 | 0.903 | 0.911 | 0.379 |
| 5 | 0.058 | 0.682 | 0.895 | 0.906 | 0.912 | 0.544 |
| 10 | 0.239 | 0.712 | 0.906 | 0.915 | 0.918 | 0.708 |
| 25 | 0.489 | 0.900 | 0.921 | 0.922 | 0.933 | 0.912 |
| 50 | 0.603 | 0.926 | 0.937 | 0.937 | 0.953 | 0.935 |
| num cases | 57 | 56 | 165 | 79 | 100 | 457 |

### Discussion

Other algorithms may assess phenotypic similarity compared to a reference set of Mendelian conditions. This may need expert and often manual curation. It may also report performance using simulated or small cohorts of real-world data (PMID 19800049, 26192085, 29997393, 29360927, 29980210). Even when using real patient data, cohorts may be part of gene discovery programs or cases from the literature, which include deep, high-quality phenotyping by genetic specialists and may not always translate to the clinical information provided to genomics testing laboratories performing case analysis (e.g., Xrare or LIRICAL). The tool described in this example may combine curated datasets of gene-phenotype associations and real-world data to rank the likelihood of a match between a gene and an individual's phenotype, with high precision and independently of variant characteristics. The tool may only consider the gene and may not consider inheritance patterns or mechanism of disease of variants associated with the gene disease pair, allowing information to be easily integrated from different sources.

Incorporating real patient data may remove the bias toward those individuals with milder disease or disorders with variable expressivity that may be easily missed by other tools that expect perfect matches with the initial cohorts of selected individuals reported in the literature. This may be consistent with the findings of AI-MARVEL (Mao D, Liu C, Wang L, et al. AI-MARVEL-A Knowledge-Driven AI System for Diagnosing Mendelian Disorders. NEJM AI. 2024 Apr. 25; 2836-9386, which is incorporated by reference herein in its entirety), which showed that an exact match with OMIM phenotypes was not critical to identify diagnostic genes. Combined with the literature surveyor tool, using the dynamic and growing internal cohort of clinical data may also allow the rescue of those genes not yet curated in OMIM or ORPHANET. This contributed to the diagnosis of an additional 2.7% of cases.

However, a concern of using real-world data is the noise from phenotypes that are not directly related to the diagnostic finding in a case. Previous work compared the phenotyping between the Deciphering Developmental Disorders (DDD) cohort and the internal cohort, showing that DDD patients had significantly fewer HPO terms on average. This was likely attributable to clinical geneticists recording HPO terms considered significant and distinctive for DDD patients, while this tool can incorporate all information available in provided clinic notes. Analysis showed that tool performance remained consistent, since it combines both curated datasets and real-world data to create the knowledge reference.

A combination of a phenotypic fit score like this tool provides and other measures of variant pathogenicity may result in a much smaller number of variants requiring review for phenotype-driven tests. This may allow scalability in case throughput and broader access for patients and may be considered an improvement over previous tools.

### Supplemental Information:

Table 2 displays information about the eight subscores used in the tool described herein. Subscores were a combination of data source and scoring algorithm. (Note: The HPOA-OMIM table contains disease-phenotype annotations. In this example, the tool compared similarity at the gene level. Therefore, all disease-phenotype annotations were flattened into gene-phenotype annotations by combining the data for all diseases on a gene.)

FIG. 3 displays a rank of the diagnostic gene by case count per gene and disease count per gene. Shown on the top is a histogram plot of the rank of diagnostic gene, separated by the bins of the internal case count. Lines representing case count (cc)=0 (301), cc=1 (302), 2≤cc≤5 (303), 6≤cc≤10 (304), and cc>11 (305) are shown. Cases assigned to genes with higher cases counts tended to have a higher rank of the diagnostic gene from the gene prioritization tool. This demonstrated the importance of well-established internal gene-phenotype annotations. Histograms are separated into cases where the diagnostic gene was never seen internally (cc=0, 301), cases where the diagnostic gene was seen once (cc=1, 302), cases where the diagnostic gene was seen 2-5 times (303), cases where the diagnostic gene was seen 6-10 times (304), and cases where the diagnostic gene was seen 11 or more times (305). Internal case count was measured by the number of single diagnosis positive cases closed by the end of the previous month, as the cases are run through the gene prioritization tool in month-wide batches. Shown on the bottom is a histogram plot of the rank of the diagnostic gene, separated by the number of diseases associated with the diagnostic gene. A histogram for cases where the diagnostic gene is associated with a single disease is indicated by 306. A histogram for cases where the diagnostic gene is associated with multiple diseases is indicated by 307. Both top and bottom plots have a log₁₀-scaled vertical axis.

### Supplementary Methods:

### Input/Output:

The gene prioritization tool returned the following outputs: (1) a probability score for each gene; (2) eight subscore values for each gene; (3) a rank for each gene; (4) an internal case count for each gene. The range of possible subscore values was [0.0,1.0] with 0.0 representing no match and 1.0 representing a perfect match. The internal case count was defined as the number of single-diagnosis positive (SDP) cases found for each gene in the date-versioned internal case table that was used to build the internal gene-phenotype annotations. The rank was defined as the fractional rank of the gene against all tested genes. The range for this value was [0.0, 1.0] where higher numbers indicated higher rank. Genes with equal probability scores got the highest possible ranks.

The RF model was trained on retrospective case data to learn the relationship between the diagnostic/non-diagnostic disease state of a gene in a patient case (the labels) and the eight subscores (the features). The model prediction was therefore an inference on which genes may lead to a diagnostic finding in a patient case. The classifier was a Random Forest (RF) model, an ensemble averaging of many decision trees trained on subsets of the data. The classifier was trained on a time-window of SDP cases from the internal dataset.

### Hybrid Relative Semantic Similarity (HRSS)

HRSS may not require exact phenotype matches to return a nonzero similarity score. If two phenotypes are on the same main branch of the HPO tree, the HRSS similarity of these two terms may be greater than zero. The “main branches” or “chapters” of the HPO tree were represented by the terms that are the child terms of HP:0000118 “Phenotypic Abnormality”, which was the root term of the relevant subontology of the HPO: hpo.jax.org/app/browse/term/HP:0000118, which is incorporated by reference herein in its entirety. Individual HRSS scores were defined between pairs of HPO terms. The HRSS subscore used in the tool was the best match average (BMA) of pairwise HRSS scores of two lists of HPO terms (the case phenotype list and the gene phenotype list).

The procedure was as follows: (1) For two lists of HPO terms of length m and n, generate a matrix of size m*n of the pairwise HRSS scores. (2) Generate a best match vector of size m+n, comprising the top score in each of m rows and n columns of the pairwise matrix. (3) Calculate the BMA via the arithmetic mean of the best match vector.

### Jaccard

Jaccard similarity may consider the number of exact matches of members of two lists of HPO terms. It may be defined as the length of the intersection of two lists, divided by the length of the union of two lists. The Jaccard subscore between a case and a gene was defined by the case terms and gene terms in the gene-phenotype annotation file.

### HPO-Based Word2Vec Model

Word2vec is a “bag-of-words” NLP method that may measure a quantitative contextual similarity between two words using word embedding. Word embedding generally refers to the process of converting a word into a vector of numbers (“word to vector”). The vector can then be compared to other vector-embedded words using standard algorithms like cosine similarity. A word2vec model may learn the context of words in a vocabulary by training a neural net. The context of a word may be defined as the words that typically appear near it in a document.

In the tool described herein, a pretrained word2vec model was used. The vocabulary for this model was the phenotype terms in the HPO. The model was trained in-house to learn the context of phenotypes from analyzing the relevant literature. HPO terms were identified. A vocabulary of 6,630 HPO terms was learned by the word2vec model. Each HPO term was represented by a vector with a length of 400.

Related words may have similar context, therefore the embeddings of related words may tend to be “close” to each other by cosine similarity. Cosine similarity served as a quantitative relationship between two words in the trained vocabulary. Table 5 shows a larger cosine similarity between two related words, “HP:0001250” (“Seizure”) and “HP:0007359” (“Focal-onset seizure”), and much lower cosine similarity between two unrelated words, “HP:0001250” (“Seizure”) and “HP:0010442” (“Polydactyly”).

TABLE 5
Cosine similarity of word embeddings (vectors) between
related and unrelated words in the HPO word2vec model.

| HPO | HPO term | HPO | HPO term | Cosine |
|---|---|---|---|---|
| term 1 | 1 name | term 2 | 2 name | similarity |
| HP: 0001250 | “Seizure” | HP: 0007359 | “Focal-onset | 0.505 |
| | | | seizure” | |
| HP: 0001250 | “Seizure” | HP: 0010442 | “Polydactyly” | 0.118 |

In the tool described herein, the word2vec similarity was implemented. The word2vec similarity between a list of case phenotypes and a list of GPAs for a single gene was defined as the cosine similarity between the average vector embeddings for each of the two lists.

### Doc2vec

Doc2vec is another “bag-of-words” NLP algorithm for semantic similarity measurements. Doc2vec can create document embeddings, where an entire document is converted into one vector (“document to vector”). All documents in the literature surveyor corpus were converted into vectors of the same size, allowing documents to be compared regardless of their original length (word count). In the implementation of the literature surveyor doc2vec model in the gene prioritization tool described herein, a document was defined in two ways: (1) a single sentence from gene-linked publications in the literature surveyor corpus; and (2) the medical text of a single patient case.

The doc2vec model was trained in-house to learn the embeddings of medical text describing patients diagnosed with genetic disease. The tool created a document embedding of case medical text via the doc2vec model. This embedded vector was then compared to documents (sentences) in the corpus linked to the genes of interest. These document-gene relationships were defined by the gene2pubmed table from NCBI. For a given gene, the doc2vec subscore was the maximum cosine similarity value of all tested documents.

The case text was preprocessed by transforming multiword phenotypes into a single “word” (e.g., token). For example, “Global developmental delay” (“HP:0001263”) became “global_developmental_delay.” The case text was subsequently embedded using DOC2VEC_MODEL.infer_vector. The document embedding vector length for case text and surveyor corpus sentences was 200.

All relevant genes in the case gene list were scored simultaneously. The gene2pubmed dataframe contained HGNC IDs linked to document keys from surveyor and was filtered down to contain only documents in the corpus that matched the relevant genes. The word embeddings from these relevant documents were loaded into the object category_docvecs and were scored against the case medical text document embeddings using cosine similarity applied by category_docvecs.most_similar. The result was a table that contained the highest cosine similarity score for each gene, e.g., the doc2vec subscores for each gene.

### More Data Dependencies

Other data dependencies included: a word2vec model trained in-house to learn the embeddings of HPO terms; a doc2vec model trained in-house using the literature surveyor tool to learn the embeddings of patient case medical text; the HPO graph; and xa_phen_filter.pkl, a table of multi-word (complex) phenotype names in the HPO network. In natural language processing (NLP), these are called “multi-word units” or “collocations”. During text encoding (word2vec and doc2vec scoring), it was desired to consider multi-word units in the HPO network. For example, the words “generalized”, “abnormality”, “of” and “skin” do not have significant meaning separately. However, “generalized abnormality of skin” may correspond to “HP:0011354”. Therefore, by scanning text for complex phenotype names and replacing whitespace with underscores (“generalized abnormality of skin” was converted to “generalized_abnormality_of_skin”) before encoded, a single token was created for a multi-word unit. This table was built by the literature surveyor tool.

Another data dependency was the mim2gene table, a key of OMIM disease ID values to gene symbols. This was used to flatten disease-level information to gene-level information. This file was provided by OMIM. Another data dependency was gene2pubmed, a regularly-updated database containing manually-curated associations between NCBI Gene and PubMed literature.

### Example 2: Literature Surveyor Tool

In this example, a toolkit was developed for managing and data mining scientific literature describing gene-phenotype relationships. This software enabled the automated downloading and formatting of publicly-available abstracts and full-text documents to construct a comprehensive corpus. This tool provided a framework leveraging natural language processing (NLP) methodologies to identify associations between genes and phenotypes, which may need to otherwise be manually discovered by literature search during clinical analysis. The literature surveyor tool performed three functions: (1) downloading and processing of all articles in the corpus; (2) calculating gene-phenotype annotations found in the corpus; and (3) training a doc2vec model to learn and generate embeddings from gene and phenotype-rich articles within the corpus. The sources of documents comprising the corpus included MEDLINE/Pubmed, PubMed Central (PMC) articles from the National Center for Biotechnology Information (NCBI), and GeneReviews.

The literature surveyor tool comprised a Python-based toolkit used for managing and data mining literature about gene-disease relationships. This software included functionality for automated aggregation and formatting of publicly available abstracts and full-text documents. A framework was also provided that enabled the use of novel natural language processing (NLP) methodologies to help identify genes that may be associated with certain phenotypes.

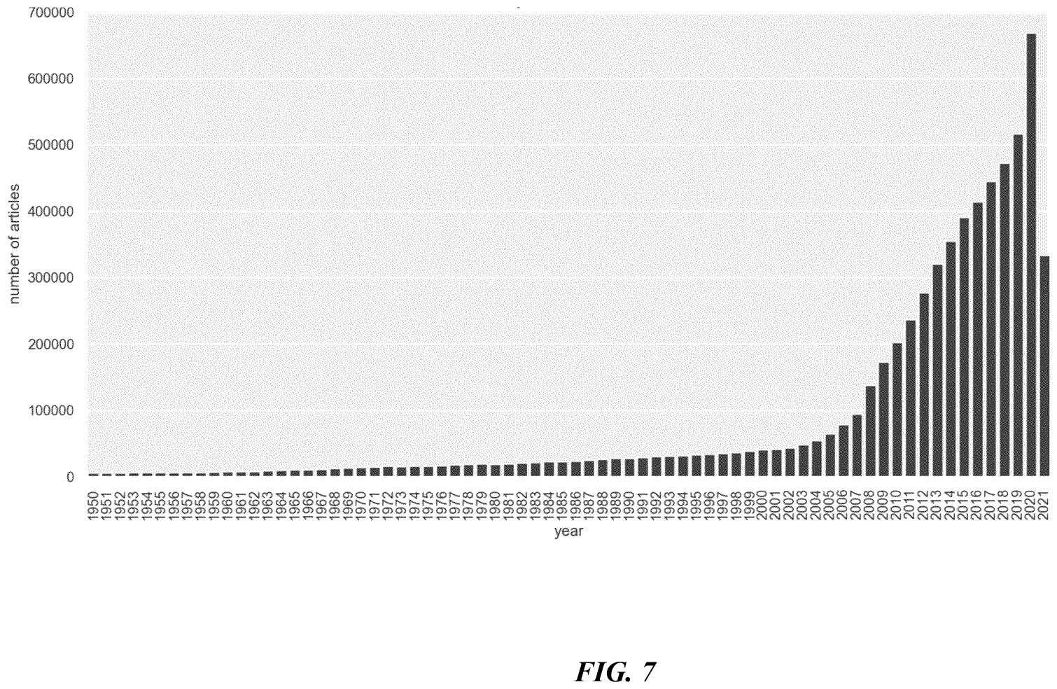

A significant majority of scientific knowledge is stored in text format, and the number of articles published annually is increasing. The literature surveyor tool stored and accessed approximately 7.2 million PMIDs downloaded from the open-access (OA) subset (last updated Jun. 4, 2021). With around 691,000 PMIDs published in 2020 alone (FIG. 7), any non-automated processing of this information is infeasible. FIG. 7 shows the number of PMIDs every year between 1950 and 2020.

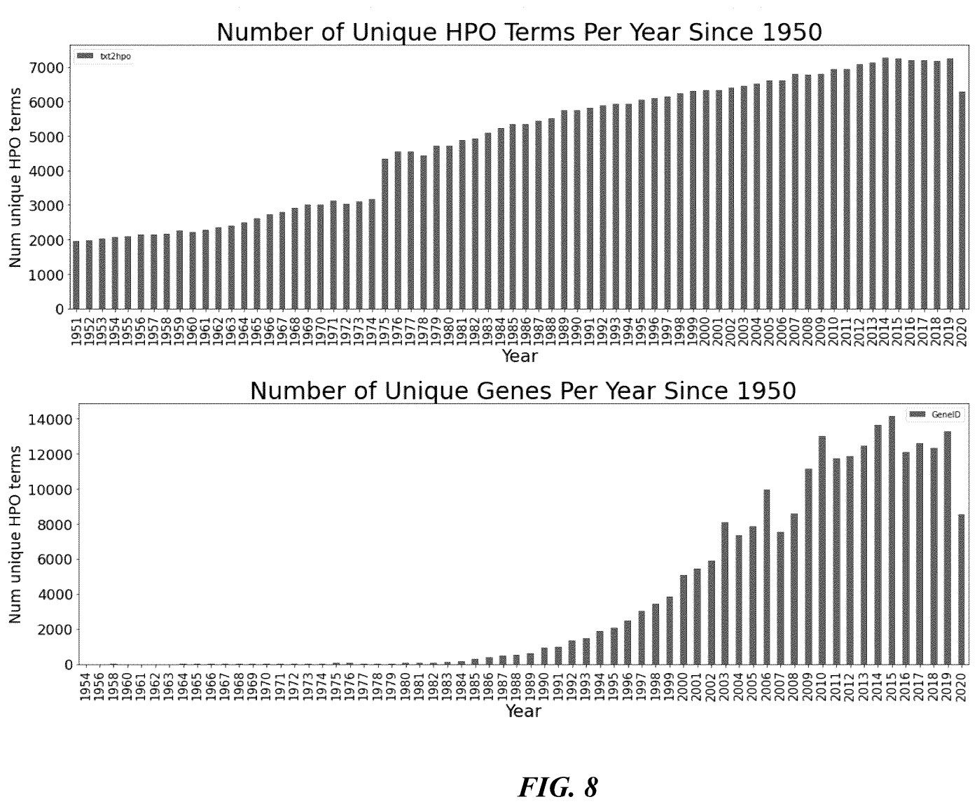

Additionally, by automating this process, it was possible to catalog emerging phenotype information and generate meaningful insight into new gene-disease associations. FIG. 8 shows mentions of unique HPO terms and gene symbols between 1951 and 2020.

The literature surveyor software can be downloaded via github and installed to a virtual environment using the following commands:
git clone https://github.com/GeneDx/surveyor.git
cd surveyor
pip install -r requirements.txt
pip install .
3 “Core” Functions

This section outlines the three “core” functions of the literature surveyor software. These include the downloading and processing of all articles from the OA subset (and from purchased articles stored in OSCAR), the calculation of normalized TF-IDF scores for all gene-HPO combinations, and the training of a doc2vec model. Though there are several other stand-alone functions that performed a variety of useful tasks for querying public databases and literature, these were the three needed to run the main functionality of the literature surveyor tool. Table 6 provides a summary of these modules.

TABLE 6
Three “core” function of the literature survey tool.

| Process | Module(s) |
|---|---|
| Download/parse/merge all articles | update_all_sources |
| Generate tf-idf format | make_tfidf_output |
| Generate input for doc2vec and train model | preprocess; train_doc2vec |

### Download/Parse/Merge Articles:

The update_all_sources command served as a main component to sequentially run the sync_all, parse_all, and merge data modules. This process required a considerable amount of time and space if implemented for the first time (several hours and ˜0.75 Tb). Additionally, the parse_all module of the code incorporated the parsing of internal PDF documents. This required that a Grobid-OCR server was running. Grobid requires JVM 8 and may be downloaded/installed using the following commands:
wget https://github.com/kermitt2/grobid/archive/0.6.1.zip
unzip 0.6.1.zip
./grobid/gradlew clean install

Then, the Grobid server may be started:
./grobid/gradlew run

It was necessary to ensure that locally-stored articles (PDF format) were stored in the ˜/.surveyor/downloads/article_pdfs/. With the server running, you may perform the following all-encompassing command to gather/parse/merge all of the articles necessary to run the literature surveyor tool.
cd surveyor
python3 surveyor/modules/data_manager.py update_all_sources

This resulted in the creation of a large pickle file (merged_data.pkl) stored in the ˜/.surveyor/parsed/output/folder. The format for this file was a data frame with the following column information: section description (intro, methods, etc.), text, PMID, txt2hpo terms identified, PMC, and publication date.

Generate the tf-idf Output:

This process took the merged_data.pkl file and generated the normalized TF-IDF scores per HPO-gene combination. This file may subsequently be used by other software (e.g., the gene prioritization tool). Once the update_all_sources command was successfully run and the merged_data.pkl file generated, the following command may be run:
python3 surveyor/modules/data_manager.py make_tfidf_out

This command called upon the modules/stats.hpo_gene_tfidf( ) module to generate the normalized TF-IDF scores per HPO-gene combination. Initially, the software calculated the raw tf-idf scores for a gene x HPO term combination using the following formula:

$$raw tf - {idf}_{ij} = {n}_{ij} * log \frac{g}{{N}_{j}}$$

In this formula, i refers to a gene, j represents an HPO term, n represents the number of pmids per HPO-gene combination, g represents the total number of unique gene ids, and N represents the number of genes per HPO term. Subsequently, the software normalized the scores as per the Maximum TF normalization method outlined in Subsequently, the software normalizes the scores as per the Maximum TF normalization method outlined in (nlp.stanford.edu/IR-book html/htmledition maximum-tf-normalization-1.html). This was done using the following formula:

$$normalized tf - {idf}_{ij} = α + ( 1 - α ) * \frac{{n}_{ij}}{{t}_{i}} * log \frac{g}{{N}_{j}}$$

In this formula, t represents the maximum term frequency per gene i, and alpha (Table 7) represented a smoothing parameter between 0 and 1. The output of this command was saved in the following file: ˜/.surveyor/parsed/output/tfidf_gene_hpo_annotation.tsv. The format of the file resembled the following:
HP:0001894 FBXO27 4.277349472802073
HP:0005506 FBXO27 3.0423165933128713
HP:0031273 FBXO27 1.5537710076996434
HP:0001894 SH2B3 0.8846686841002591

### Generate Tokenized Input File for Doc2Vec Training:

This section outlines the commands that were necessary to train a doc2vec model on the literature. This process required that the merged_data.pkl be generated via the update_all_sources modules, as well as a pickle file named xa_phen_filter.pkl. This file was a dataframe that needed to be generated by querying XA for the primary_indication and past_medical_history fields of a pre-specified subset of (or all) patients. In XA, the following commands needed to be issued to select this information for all patients:
SELECT primary_indication.past_medical_history FROM cases;
This command may be directly saved to a dataframe if using the psycopg2 and pandas libraries by issuing the pandas.read_sql( ) command. Alternatively, the results of this command in PSQL may be output to a file using \copy or a combination of \i and †o (if the above command was saved as a .sql file). By running the command below, the primary_indication and past_medical_history fields may be concatenated and parsed for punctuation, capitalization, and a pre-defined dictionary of “non-phenotypic” terms (/surveyor/internal/data/non_phenos.py). This file needed to be stored in the
˜/.surveyor/internal/data/folder.
cd surveyor/internal/

Then, in a Python terminal:
import utils
utils.build_xa_phenos_filter([path_to_xa_dataframe], [frequency_threshold]

The frequency threshold (Table 7) referred to the minimum number of mentions that a phrase must have in order for it to be included in the xa_phen_filter.pkl file. This output file was stored in the ˜/.surveyor/downloads/common folder. Once this file was generated and saved, the following command may be run from the command line:
python3 surveyor/modules/corpus_builder.py preprocess

The output of this process was stored in the following location:
˜/.surveyor/parsed/output/merged_data_d2v_preprocess.pkl. The format for this file is a data frame with the following column information: text, PMID, taxonomy ID, GeneID (NCBI), number of genes identified in the Text field, phenotype associated and extracted from the Text field using txt2hpo, and HPO terms identified from Text using txt2hpo.

Once the file above has been generated, the command below may be run to train the model:
python3 surveyor/models/text2gene_d2v.py

This command utilized a library to train a doc2vec model using the command above. This stored a “_self-assessment.pkl” file in the ˜/.surveyor/models/text2gene_d2v/ directory. The fields contained within this file are listed as follows: entry identifier (“tag”), rank, P-value, list of tokens (“text”), and length of the token list.

### Detailed Article Information Used by the Literature Surveyor Tool:

This section provides a description for the types of records and articles that were downloaded and processed for the literature surveyor tool. Though the initial gathering of this information required a considerable amount of time and memory, subsequent updates to the downloaded dataset were fast and efficient. This synchronization process was due to a comparison that the literature surveyor tool made between the existing catalog of downloaded articles and those stored in Pubmed.

### Baseline Set of MEDLINE/Pubmed Citation Records:

This baseline was updated in December of each year. All files (alongside md5 checksums) were downloaded via FTP and stored in ˜/.surveyor/downloads/medline/.

Daily Updated List of Citation Records from MEDLINE/Pubmed:

The PubMed Baseline Repository was updated daily. The literature surveyor tool updated the existing versions of these files which included revised and deleted citations. This process was fairly quick depending on the size of the update.

PMC Articles Stored in oa_package Directory from NCBI:

These files were stored as archives (.tar.gz) and contained NXML versions of the article, any associated images, supplementary materials, pdf versions of the article (if available), and videos for streaming (if available). Each of these files was stored in the ˜/.surveyor/downloads/pmc directory.

PMC Articles in Manuscript Form from NCBI:

These files (XML) pertained to the entire author manuscript collection which the literature surveyor tool downloaded via PMC's FTP service. These files were updated every Monday and Thursday.

GeneReviews from NCBI:

These files pertained to all GeneReviews chapters which were stored as pickle (.pkl) files in ˜/.surveyor/downloads/genereviews/. Each entry within the pickle files contained 4 fields (chapter text, pmid, section identifiers, and PMC).

### Internal Article PDFs:

The literature surveyor contained the ability to parse internal PDF files stored in the ˜/.surveyor/downloads/article/pdfs folder. Processing the internal files required that a grobid server was actively running (described above). The information within each PDF file was extracted and saved in three separate formats: XML (˜/.surveyor/parsed/article_xmls/), JSON (˜/.surveyor/parsed/article_jsons/), and PKL (˜/.surveyor/parsed/article_pkls/).

### Literature Surveyor Tool Update Schedule:

All articles were stored locally on the ds-nas and were updated biweekly (via update_all_sources). Additionally, all tf-idf output and doc2vec models were updated immediately following the synchronization, parsing, and merging of all downloaded literature.

TABLE 7
Default parameters described in this example.

| | Default | | Implemented |
|---|---|---|---|
| | value | Description | in . . . |
| alpha | 0.4 | Smoothing term used | Generate tf- |
| | | to dampen the | idf output |
| | | contribution made | |
| | | by term frequency | |
| Frequency | 49 | Minimum number of | Doc2vec |
| threshold | | mentions that a phrase | training |
| | | must have in order for | |
| | | it to be included in | |
| | | the xa_phen_filter.pkl | |
| | | file | |

### Example 3: Multiscore Tool

Reporting in genomic testing may be phenotype driven; therefore, identifying variants in genes with a phenotypic fit may be fundamental. Current approaches may determine this measure by manual review by a clinical analyst and may be assisted by a pipeline search (e.g., “Phenotype” search) that assesses Human Phenotype Ontology (HPO) curated phenotypic data only. However, new disease genes, not yet curated in Online Mendelian Inheritance in Man (OMIM) have no phenotypic information and therefore may pose challenges.

Using systems and methods of the present disclosure, a multiscore tool was developed to out-perform current approaches in specificity, consistency, and scalability. Using systems and methods of the present disclosure, a multiscore tool was developed that combines multiple measures of phenotypic similarity informed by external and internal data sources and powered by machine learning (ML) to score the similarity between a phenotypic profile of an individual (including a list of HPO terms plus free text in the form of clinical descriptions) and phenotypes associated with a gene. A goal may be to provide reliable predictions for prioritizing variants for exome or genome-based testing based on case-level phenotypic information assisting the variant filtering.

The multiscore tool may have various data dependencies, and uses scorers and phenotypic information to yield predictive multiscore values. For example, a year of retrospective cases in a disease phenotype database (Disease Phenotype Resource (DiPR)) were scored using Multiscore.

The term “Annotations”, as used herein, generally refers to DiPR (internal) and Surveyor (literature) based gene-phenotype associations which provide inputs for the Hybrid Relative Semantic Similarity (HRSS), word2vec, and Jaccard Scorers.

The term “DiPR”, as used herein, generally refers to Disease Phenotype Resource, a generated data table of Exome Sequenced cases, which may be updated periodically (e.g., monthly) based on date of case closed.

The term “false negative”, as used herein, generally refers to a patient case in the analysis where the diagnostic variant may be filtered out per threshold rules. In general, a false negative refers to a test result where the test did not capture a positive result. False negatives may be considered a highly costly error in medical diagnostics.

The term “feature score”, as used herein, generally refers to an input to the Multiscore Classifier model. These may be expressed as floating point numbers between 0 and 1, inclusive, that represent the similarity between input phenotypic data and each gene. For each gene, there is one feature score per Scorer.

The term “flatten”, as used herein, generally refers to an approach in which disease data is collapsed onto genes and stored at the gene level, including OMIM disease data. In production, only the gene HGNC ID of the variant list is entered in the request payload. In this context, disease-phenotype annotations and gene-phenotype annotations are equivalent.

The term “Human Phenotype Ontology (HPO)”, as used herein, generally refers to a graph of standardized phenotypic abnormalities encountered in human disease (hpo.jax.org/).

The term “Multiscore”, as used herein, generally refers to a software tool combining multiple measures of phenotypic similarity scoring to predict overall relevance of variant data in an Exome or Genome test.

The term “Multiscore model”, as used herein, generally refers to a machine learning classifier, in which a machine learning based algorithm combines multiple features to yield a combined predictive score. In Multiscore, the model gives the final score for the similarity between phenotypic data and a gene. For example, a Multiscore model may comprise a Random Forest Classifier. The ML model may not understand gene-phenotype relationships, only how similarity score features from the Scorers may relate to identification of a molecular diagnosis (e.g., “cat1” result). The model is comprise a set of files which may contain a binary model file, index files, overflow vector files, and more. The model input may comprise eight feature scores. The model output may comprise a final score (e.g., Multiscore value) describing the match between a case and a gene.

The term “Online Mendelian Inheritance in Man (OMIM)”, as used herein, generally refers to an online resource for inherited diseases, providing known disease-phenotype associations.

The term “Phenopy”, as used herein, generally refers to a phenomics similarity scoring software tool.

The term “pipeline search variants”, as used herein, generally refers to variants that are found to be of interest and to be interrogated for manual review.

The term “Scorer”, as used herein, generally refers to combinations of scoring algorithms and source data that produce a similarity score comparing a gene to a phenotype list (hrss_hpoa, hrss_dipr, hrss_lit, w2v_dipr, w2v_lit, jaccard_dipr, jaccard_lit) or a gene to free text (doc2vec). The similarity scores may be feature scores for the Multiscore model.

The term “Surveyor”, as used herein, generally refers to a literature mining software tool.

The term “test data”, as used herein, generally refers to held-out test cases scored for Multiscore software studies.

The term “training data”, as used herein, generally refers to past cases used to train the Multiscore model.

The term “txt2hpo”, as used herein, generally refers to a Natural Language Processing (NLP) software tool used for extracting HPO terms from text.

The term “XomeAnalyzer”, as used herein, generally refers to a data processing pipeline for annotating and filtering variant data from Exome and Genome tests.

### Abbreviations:

DiPR: Disease Phenotype Resource
ES: Exome Sequencing
FN: False Negative
GS: Genome Sequencing
HGNC: HUGO Gene Nomenclature Committee
HPO: Human Phenotype Ontology
HRSS: Hybrid Relative Semantic Similarity
ML: Machine Learning
NLP: Natural Language Processing
OMIM: Online Mendelian Inheritance in Man
POC: Proof-of-concept
RF: Random Forest
SDC1: Single diagnosis, Category 1. Refers to cases in DiPR where only one gene is given a “Causative Mutation” relevance.
XA: XomeAnalyzer

In the downstream processing of Exome Sequencing and Genome Sequencing (ES/GS) samples, XA returns pipeline search variants in a proband based on classification status, population frequency, inheritance pattern, variant type, phenotype association, and more. Multiscore fills the unmet need of reducing the number of pipeline search variants that require manual review, by scoring based on phenotypic similarity and then de-prioritizing those that are unlikely to lead to diagnosis. Multiscore differs from the current Phenotype pipeline search approach because it can apply across all pipeline searches and considers a richer truth-set data source than the Phenotype pipeline search.

Multiscore relies on several input source data dependencies, including:
gene-phenotype annotations from internal ES case data extracted from the DiPR dataset, updated periodically (e.g., monthly).
gene-phenotype annotations extracted from literature mining by Surveyor, updated periodically (e.g., biweekly).
gene-phenotype annotations extracted from OMIM-ORPHANET annotations (purl.obolibrary.org/obo/hp/hpoa/phenotype.hpoa).
a word2vec natural language processing (NLP) trained on HPO terms and committed to Phenopy. This is a static model and does not update with changes to the HPO.
a doc2vec NLP model updated bi-weekly by Surveyor.

The Multiscore app receives a request payload (see below) containing a case identifier, a list of HPO terms, free text describing the patient, and a list of HGNC IDs, and computes phenomics-driven similarity computations. Multiscore runs the request data payload through eight subscore-generating tools called Scorers. Each Scorer contains a unique combination of dependency data source and scoring algorithm. Each Scorer generates a similarity score between the phenomics data (HPO terms and case text) and each gene in the list of gene IDs. For n Scorers and m gene IDs, Multiscore generates a table of n*m similarity scores.

In an example, the number of Scorers is eight (n=8). More Scorers may be added to improve predictive performance. Multiscore has been designed to be extensible and modular, where feature generating Scorers can be added, tuned, or removed.

The similarity scores from each Scorer are treated as feature scores and fed into a Random Forest (RF) classifier (the Multiscore Classifier model) that generates the final scores (e.g., the Multiscore Values). There is one Multiscore Value for each gene. The Multiscore model has been trained on retrospective case data to learn which subscore Scorer values are associated with diagnostic genes. The Multiscore Value is the model's prediction on which genes may lead to a diagnostic finding.

For n Scorers, a single final score, and m gene IDs, the response data from Multiscore can be viewed as a final table of (n+1)*m similarity scores. An example result is given in Table 8. The actual response payload is in json format.

TABLE 8
Example result of Multiscore query of m-many gene IDs

| | Multiscore | Scorer 1 | Scorer 2 | Scorer 3 | | Scorer n |
|---|---|---|---|---|---|---|
| Gene ID | Value | Value | Value | Value | . . . | Value |
| GENE₁ | MV₁ | GENE₁SV₁ | GENE₁SV₂ | GENE₁SV₃ | . . . | GENE₁SVₙ |
| GENE₂ | MV₂ | GENE₂SV₁ | GENE₂SV₂ | GENE₂SV₃ | . . . | GENE₂SVₙ |
| GENE₃ | MV₃ | GENE₃SV₁ | GENE₃SV₂ | GENE₃SV₃ | . . . | GENE₃SVₙ |
| . . . | . . . | . . . | . . . | . . . | . . . | . . . |
| GENE_{M} | MV_{M} | GENE_{M}SV₁ | GENE_{M}SV₂ | GENE_{M}SV₃ | . . . | GENE_{M}SVₙ |

Multiscore is queried by the XomeAnalyzer via a FastAPI app to score the similarity between the phenotypic profile of an ES or GS proband and all the pipeline search variants on that case. At present, Multiscore only considers the gene related to each variant and does not consider inheritance, mechanism of disease, or the molecular strength (pathogenicity) of the variant.

Multiscore may only reliably score disease genes with at least 1 SDC1 case in DiPR. Services calling Multiscore may only expect to receive actionable data on validated disease genes; any other status such as “candidate” may be considered suspect for downstream use.

Multiscore is expected to rank the diagnostic gene in the top 50% of all scored genes in 97% of cases. This may lead to 50% of genes considered for analysis to be thrown out, and the diagnostic gene may be deprioritized in 3.2% of cases. This is based on a analysis of retrospective cases, which is described herein. These numbers may be based on an embodiment in which Multiscore may be the only filter in the ranker that prioritized and hid some of the XA pipeline search variants. The variants may be ranked by phenotype match without any filtering of the pipeline variants.

A list of HPO term codes may be used for describing a patient. All terms may be related to Mendelian disease and children of the term “HP: 0000118” aka “phenotypic abnormality” hpo.jax.org/app/browse/term/HP: 0000118.

Free text may be used for describing a patient. In Multiscore training, testing, and development, free text describing a patient from the XA fields was used: cases.primary_indication, cases.past_medical_history, and cases.differential_diagnosis.

A set of genes may be used, such as a list of HGNC IDs that the input phenomic information is scored against.

For each gene in the request payload, a Multiscore value is returned as a probability, and a score for each Scorer is returned in the scores structure, with the names of each Scorer given as property1, property2, etc., with the Scorer values for each. The contents of scores is for testing and development purposes. The range of possible Scorer values is [0,1] (inclusive), with 0 representing no match and 1 representing a perfect match. The internal_case_count is defined as the number of DiPR single-diagnosis cat1 (SDC1) cases found for each gene in the date-versioned DiPR case table that was used to build the DiPR-based gene-phenotype annotations. The final value in the response schema is the fractional rank of the gene against all tested genes. The range for this value is [0.0, 1), where lower numbers indicate higher rank.

Eight scoring algorithms (e.g., Scorers) generate feature scores that are fed into an ML classifier to give a final score (e.g., the Multiscore result). The classifier comprises a Random Forest (RF) model, an ensemble averaging of many decision trees trained on subsets of the data. The source for the RF classifier is the Python package scikit-learn, (scikit-learn.org/stable/index.html) and uses the method sklearn.ensemble.RandomForestClassifier. Note that other ML models are possible and Multiscore has been designed with the option for replacement of the classifier. The classifier is trained on closed cases from the internal Disease Phenotype Resource (DiPR) data set.

A DiPR gene-phenotype annotation file data source may be used. The original source for the internal GDx data is a comprehensive structured dataset of clinical data describing probands suspected of rare disease and referred for clinical exome sequencing called the Disease Phenotype Resource (DiPR). The gene-phenotype relationships from all single-gene diagnosis Causative Mutation category 1 (SDC1) DiPR cases are compiled into a table with the following columns: HPO term, HGNC ID, and Term Frequency-inverse Document Frequency (TF-iDF). All phenotypes that have been seen across all SDC1 cases for each gene are stored in this table. These gene-phenotype relationships are called annotations; in this case they are called the DiPR-based annotations. NOTE: the TF-iDF values for gene-phenotype associations are not used in Scorer calculations at this time.

An OMIM disease-phenotype association file data source may be used. The Online Mendelian Inheritance in Man (OMIM) resource provides a downloadable data table phenotype.hpoa (purl.obolibrary.org/obo/hp/hpoa/phenotype.hpoa) of HPO annotations (HPOA) for all Mendelian diseases found in humans. OMIM-HPOA gene-phenotype annotations are built from this file. OMIM also provides the structure of the HPO directed graph (HPO network) in downloadable a text file (purl.obolibrary.org/obo/hp.obo). Phenopy downloads these files upon initialization including when Multiscore loads Phenopy methods. Since Multiscore currently operates at the gene level, all diseases are flattened onto their respective gene and the gene-phenotype annotations represent all phenotypes listed on all diseases for a gene.

A literature disease-phenotype annotation file data source may be used. The Surveyor package (github.com/GeneDx/surveyor) was used to mine the relevant published literature and produces a gene-phenotype annotation file in the same format as the DiPR annotation above. This table is also called the literature-based annotations.

A literature-based doc2vec model file data source may be used. The surveyor package (see above) generates a doc2vec model of gene-phenotype associations trained on free text describing Mendelian disease cases found in the relevant literature. This model is updated periodically (e.g., bi-weekly) by Surveyor, and Multiscore employs the most recent model.

There may be secondary literature-based data dependencies from Surveyor. Several other supporting files are supplied by Surveyor. For example, gene2pubmed.gz is an index of literature in the Surveyor literature corpus, including a key of PubMed ID values to gene symbols. As another example, mim2gene.pkl is a key of OMIM disease ID values to gene symbols, which is used to flatten disease-level information to gene-level information. As another example, xa_phen_filter.pkl is a table of multi-word (complex) phenotype names in the HPO network. In natural language processing (NLP), these are called “multi-word units” or “collocations”. During text encoding (word2vec and doc2vec scoring) multi-word units were considered in the HPO network, for example, the words “generalized”, “abnormality”, “of” and “skin” do not have significant meaning separately, however “generalized abnormality of skin” is the name of “HP: 0011354”. Therefore, by scanning text for complex phenotype names and replacing whitespace with underscores (“generalized abnormality of skin” is converted to “generalized_abnormality_of_skin”) before encoding, a single token can be created for a multi-word unit.

Updates to DiPR Scorer annotations may be made. The Scorer annotations are updated using a manual data pipeline, however efforts are made to automate these processes in a Dagster pipeline. Multiscore uses pydantic conventions for data dependencies and expects names based on environment variables committed to the Multiscore code repository (e.g., see multiscore.k8s.values.yaml). For example, the DiPR based gene-phenotype annotations file defined is set by the environment variable MULTISCORE_DATA_DISEASE_TO_PHENOTYPE_CUSTOM which translates to DISEASE_TO_PHENOTYPE_CUSTOM in python. The dependency files are uploaded to the ML Platform artifact repository at the expected locations and a history of dependency files is stored on servers for historical analysis and explainability. The Phenopy OMIM files phenotype.hpoa and hp.obo are also refreshed on a schedule. Phenopy downloads these files via phenopy.config.download_resource_files( )

A Hybrid relative semantic similarity (HRSS) algorithm may be used for scoring. HRSS is a similarity metric combining information content and HPO graph structure. HRSS is fully described in Wu 2013 et al. (pubmed.ncbi.nlm.nih.gov/23741529/), which is incorporated by reference herein in its entirety. Unlike Jaccard similarity (see below), HRSS does not require exact phenotype matches to return a nonzero similarity score. As long as two phenotypes are on the same main branch of the HPO tree, the HRSS similarity of these two terms is greater than zero. The main branches of the HPO tree are all the children terms of HP: 0000118 “Phenotypic Abnormality” which is the root term of the phenotypic abnormality subontology of the HPO:

hpo.jax.org/app/browse/term/HP: 0000118. The HRSS similarity for two lists of HPO terms of length m and n is calculated by generating a matrix of all m*n pairwise HRSS scores and taking the average of the top score in each column and in each row aka a best-match average. The HRSS score between a case and gene is defined as the HRSS list similarity between all case terms and all gene terms in the gene-phenotype annotation file. HRSS in Multiscore is implemented using the Phenopy method phenopy.score.Scorer.score_hpo_pair_hrss.

A Jaccard similarity algorithm may be used for scoring. Jaccard similarity is defined as the length of the intersection of two sets divided by the length of the union of two sets. Unlike HRSS similarity (see above), Jaccard considers the number of exact matches in two lists. The Jaccard score between a case and a gene is defined by the case terms and gene terms in the gene-phenotype annotation file. Jaccard similarity in Multiscore is implemented using the Phenopy method phenopy.score.Scorer.score.

An HPO-based word2vec model may be used for scoring. Word2vec is an NLP method that measures a quantitative contextual similarity between two words using word embedding. Word embedding is the process of converting a word into a vector of numbers (“word to vector”). The vector can then be compared to other vector-embedded words using standard algorithms like cosine similarity. A word2vec model must be trained to learn the context of words in a vocabulary. The context of a word is defined as the words that typically appear near it in a document. A word in the trained vocabulary does not have to be a real word; since word2vec doesn't understand the English language before training a model, it can be a new word. In our example case of a model trained for use in a disease phenotype space, “HP: 0001290” can be treated as a word. This is the HPO term for “Generalized hypotonia”. The training is performed using a neural network.

In Multiscore, a pretrained word2vec model committed to Phenopy is used. This is a word2vec model of HPO term word embeddings trained on patient descriptions in the relevant literature. HPO terms were identified using the GDx developed software tool txt2hpo (github.com/GeneDx/txt2hpo) and the model subsequently learned the semantic context of these terms. In other words, once an HPO term was identified in the literature text, the words nearby the HPO term were collected, and over the corpus of relevant literature, the word2vec model learned which words tended to appear near which HPO terms. A vocabulary of 6,630 HPO terms was learned by the word2vec model. Each HPO term is represented by a vector with a length of 400.

Related words tend to have vectors “close” to each other, which can be measured by cosine similarity. The cosine similarity of the vectors of two related words is larger than two unrelated words. See Table 2 for the cosine similarity between two related words, “HP:0001250” (“Seizure”) and “HP: 0007359” (“Focal-onset seizure”), and much lower cosine similarity between two unrelated words, “HP: 0001250” (“Seizure”) and “HP: 0010442” (“Polydactyly”).

TABLE 9
Cosine similarity of word embeddings (vectors)
between related and unrelated words in the
HPO word2vec model committed to Phenopy.

| HPO | HPO term | HPO | HPO term | Cosine |
|---|---|---|---|---|
| term 1 | 1 name | term 2 | 2 name | similarity |
| HP: 0001250 | “Seizure” | HP: 0007359 | “Focal-onset | 0.505 |
| | | | seizure” | |
| HP: 0001250 | “Seizure” | HP: 0010442 | “Polydactyly” | 0.118 |

In Multiscore, a word2vec similarity is implemented. The word2vec model is a binary file that was trained and has been committed to the Phenopy package. Word vectors are loaded within initialization of the Python class object phenopy.score.Scorer. The word2vec similarity result is calculated in the Phenopy method phenopy.score.Scorer.score, including taking the cosine similarity between the average vector of two-word lists.

Using the example of Multiscore to explain further, each word in two term lists (the case terms and the gene terms from the gene-phenotype annotations) is embedded and the average vector of each term list is found. Then the cosine similarity is taken between the average vector of the case terms and the average vector of the gene terms.

A Doc2vec model may be used for scoring. Doc2vec is another NLP algorithm for semantic similarity measurements. As the name implies, doc2vec creates document embeddings, where an entire document is converted into one vector (“document to vector”). All documents in the literature corpus are converted into vectors of the same size which allows documents to be compared regardless of their original length (e.g., word count).

The surveyor package mines relevant literature for gene-phenotype associations and outputs a doc2vec model. The model contains a document embedding (vector) for each document (publication) in the corpus of literature. Multiscore creates a document embedding of queried case text, and then this embedded vector is compared to all publications the corpus pertaining to the queried case genes. The top score for each gene represents the doc2vec Scorer value for that gene. Consider, for example, a case that contains the gene HGNC: 21316 (ANKRDJJ) with 100 publications linked to HGNC: 21316 (ANKRDJJ) in the surveyor corpus. The vector of embedded case text is scored against the vectors for all 100 embedded publications linked to HGNC: 21316, and the top similarity score is the doc2vec Scorer value for HGNC: 21316.

In Multiscore testing and development, the case text for an ES case is the sum of the ExomeAnalyzer Database fields cases.primary_indication, cases.past_medical_history, cases.differential_diagnosis. The doc2vec similarity scores are calculated in the method multiscore.doc2vec.d2v_similarity. The doc2vec model object doc2vec_model is defined. A new model file is provided by Surveyor periodically (e.g., biweekly).

The case text is preprocessed in multiscore.text.process_text which formats the text and transforms multiword phenotypes into a single “word” so it is treated as a single token (see above), e.g., “Global developmental delay” (“HP: 0001263”) becomes “global_developmental_delay.” The case text is subsequently embedded into a vector using DOC2VEC_MODEL.infer_vector. The document embedding vector length for case text and surveyor corpus publications is 200.

All relevant genes in the case gene list are scored simultaneously. The gene2pubmed dataframe contains HGNC IDs linked to document keys from surveyor and is filtered down to contain only documents in the corpus that match the relevant genes. The word embeddings from these relevant documents are loaded into the object category_docvecs defined by g13enism.models.keyedvectors.WordEmbeddingsKeyedVectors and are scored against the case text word embeddings using cosine similarity applied by category_docvecs.most_similar. The result is a table (pandas dataframe object) named query that contains the highest score for each gene, e.g., the doc2vec Scorer values between the case text and for each case gene.

The Scorers that generate feature scores for the classifier each represent a unique combination of source data and scoring algorithm to calculate a similarity between the phenotypic profile and the gene of interest. Table 10 provides details on all Scorers in Multiscore. Multiscore is designed with extensibility in mind; a new Scorer can be easily added by scoring the retrospective training data using the new Scorer and re-training the Multiscore Classifier Model.

TABLE 10
Description of Scorers in Multiscore. Scorers are
a combination of data source and scoring algorithm.

| | | Scoring | |
|---|---|---|---|
| Scorer Name | Data Source | Algorithm | Note |
| hrss_hpoa | HPOA-OMIM | HRSS | Case HPO terms are |
| | annotation | | compared to all gene* |
| | file | | HPO terms in HPOA- |
| | | | OMIM |
| hrss_dipr | DiPR | HRSS | Case HPO terms are |
| | annotation | | compared to all gene |
| | file | | HPO terms from SDC1 |
| | | | DiPR cases |
| hrss_lit | Surveyor | HRSS | Case HPO terms are |
| | literature | | compared to all gene |
| | annotation | | HPO terms found in |
| | file | | literature mining |
| jaccard_dipr | DiPR | Jaccard | Case HPO terms are |
| | annotation | similarity | compared to all gene |
| | file | | HPO terms from SDC1 |
| | | | DiPR cases |

*Note that the HPOA-OMIM table contains disease-phenotype annotations.

At present, Multiscore compares similarity at the gene level, therefore all disease-phenotype annotations are flattened into gene-phenotype annotations by combining the data for all diseases on a gene.

Training the Multiscore Classifier was performed as follows. The Multiscore Classifier model was trained on retrospective DiPR cases. A goal of the classifier is to analyze phenotypic Scorer values and predict whether a variant on a given gene receives a diagnostic result of “Causative Mutation” aka “category 1” or “cat1”. Mechanically this approach comprises reading the eight Scorer values and assigning a final Multiscore value. The features were Scorer values for a gene, and labels were cat1/not cat1 status (I/O). In machine learning, training on features to predict labels is called “Supervised Learning.”

The Multiscore Classifier was trained on a one-year window of SDC1 DiPR cases. In Multiscore, model training can be called a time-series experiment: the Scorer data dependencies were built using case data and literature data that were known at the beginning of the one-year window, and the training data (scorers and features) were built based on cases in the one-year window.

A detailed description of the training process follows below:

1) Build Multiscore Scorer data dependencies. Build gene-phenotype annotations for DiPR SDC1 cases. Run surveyor in a special retrospective mode called surveyor snapshot, which generates output based on literature available on or before a certain date.

2) Generate all eight Scorer values for all genes with XA variant pipeline search results for the set of DiPR SDC1 cases. These cases are the training data. The Scorer values are the features, and the cat1 (1) and not-cat1 (0) status are the labels. Feature generation resulted in 5,729,406 genes analyzed across 28,379 cases, with 4,947 positive labels aka genes with cat1 variants. The variant pipeline search results were collected using the scripts retrospectives.dipr.pipeline_var_search.py and retrospectives.dipr.pipeline_var_details.py, which respectively trigger fresh XA pipeline searches to obtain (lims_case_id, variant_id) pairs and subsequently the detailed variant data on those pairs. Due to internal data issues in XA (gene assignment etc.), variants with variant_type of CNV_dup and CNV_del were removed, and SDC1 cases where these were the diagnostic variant were removed. Scores were generated using the script multiscore.scripts.make_scores.py.

3) Train the ML Classifier on the training data. The negative labels were downsampled to create a positive-to-negative ratio of 1:2. This produces the trained ML Classifier model file. Features were generated from the scores using multiscore.scripts.make_features.py and the model file was trained and generated using multiscore.scripts.validation.py.

The FastAPI app for Multiscore returns a json of Multiscore values, Scorer values, and gene cohort size for each gene in the inbound payload. The Multiscore value is expected to accurately identify correlations between a gene and the given phenotypic information in the inbound payload: high values for close matches, and low values for unrelated gene-phenotype entries.

The Multiscore Classifier was tested and validated as follows. In order to test the above expectations, a set of retrospective SDC1 cases in DiPR were studied. Multiscore Values were generated for all case genes from diagnostic and non-diagnostic XA pipeline variants on disease genes. The case genes were ranked to understand the ability of the classifier to correctly identify diagnostic genes based on phenotypic case data. This analysis provides context for future development of filtering thresholds for Multiscore values that may lead to fewer variants sent for clinical review.

Testing the Multiscore Classifier was performed by simulating how the software runs in production. In production, phenotypic case data is scored against all pipeline variant disease genes using the temporally available data dependencies. Testing was performed on retrospective ES (DiPR) case data using the data dependencies that are available when the cases were analyzed. The cases were scored in one-month batches based on the case closed date, and each batch has temporally appropriate data dependencies. Surveyor is run periodically (e.g., bi-weekly), and the latest run from the previous month was utilized; in production the latest available surveyor model is used.

A detailed breakdown of the methods follows:

1) A total of 4,973 SDC1 DiPR cases were scored using temporally relevant data dependencies in one month batches. lims_case_id was used as the case identifier. Due to internal data issues in XA (gene assignment etc.), variants with variant_type of CNV_dup and CNV_del were removed, and SDC1 cases where these types were the diagnostic variant were removed.

2) Case data were prepared as follows: a) Case free text for the doc2vec scorer was based on the text column from the case row in DiPR. The text column is a concatenation of the XA entries cases.primary_indication, cases.past_medical_history, cases.differential_diagnosis. b) Case HPO terms were based on the hpo_terms column from the case row in DiPR. Those values are the members of set (set (xa_hpo_terms+txt2hpo)-xa_rej_hpo_terms), where xa_hpo_terms=HPO terms annotated to case by human abstractors, and xa_rej_hpo_terms=HPO terms rejected by human abstractors. c) txt2hpo=HPO terms added by analyzing above “text” column with the NLP-based tool txt2hpo (github.com/GeneDx/txt2hpo). Case genes were based on all XA pipeline search variants on Mendelian disease genes (gene has is_mendelian tag plus at least one disease with is validated status in geneMB).

3) The score of the diagnostic gene (variant) was compared to all (non-diagnostic) case genes.

A total of 4,973 SDC1 cases were evaluated in Multiscore. The performance was evaluated based on the proposed usage of Multiscore phenotypic fit data. In XA, Multiscore helps triage variants in a case that are more likely to lead to a diagnosis. Some variants in the pipeline search results (the case genes) may be filtered out, which leads to higher throughput due to less time spent per case. Filtering out pipeline search results, however, may introduce a risk of removing some case genes that could lead to diagnosis. A False Negative (FN) result was defined as a case where the previously assigned diagnostic variant is filtered by some chosen method.

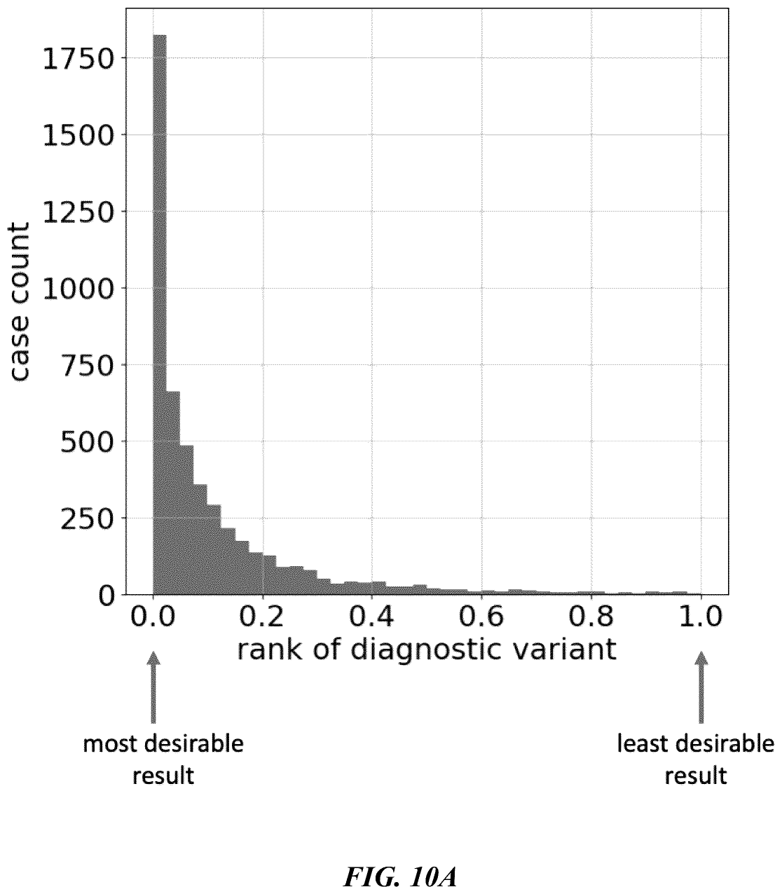

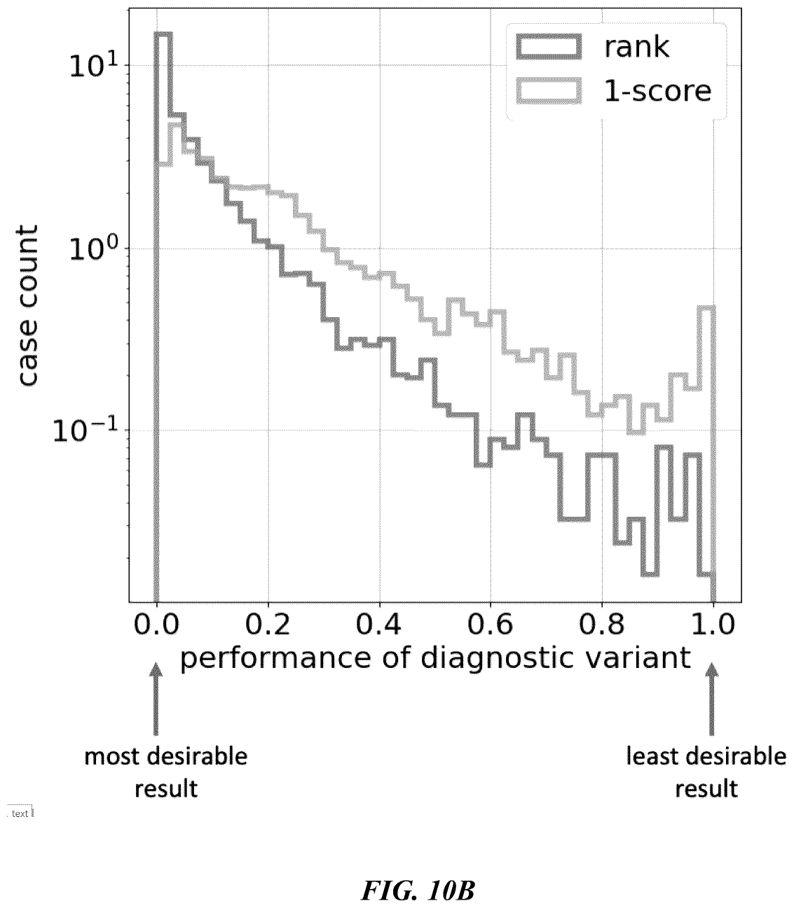

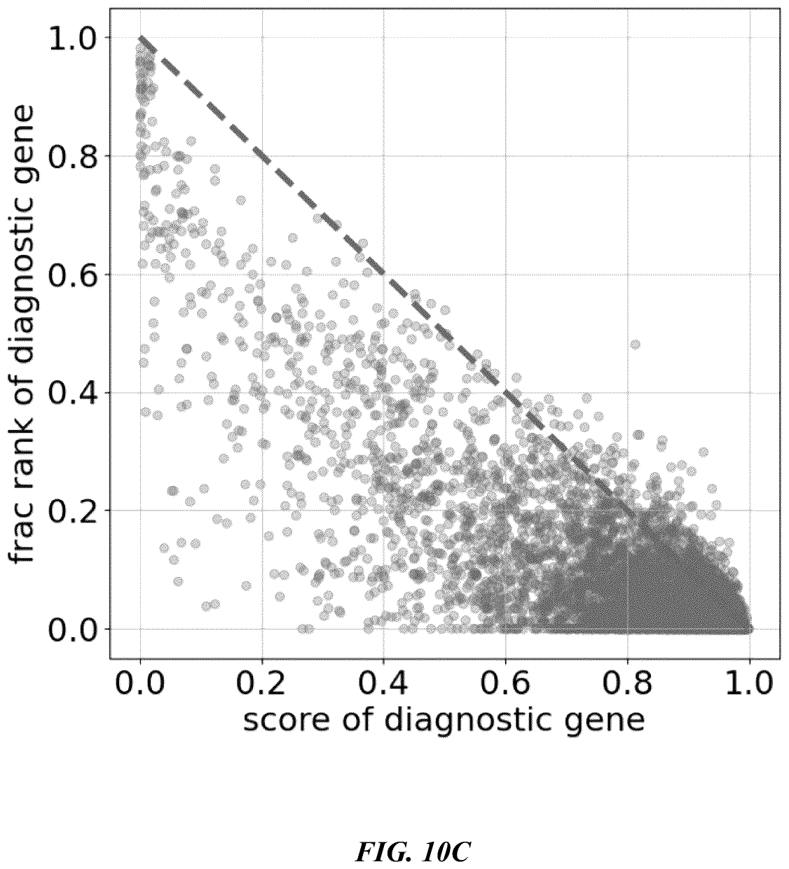

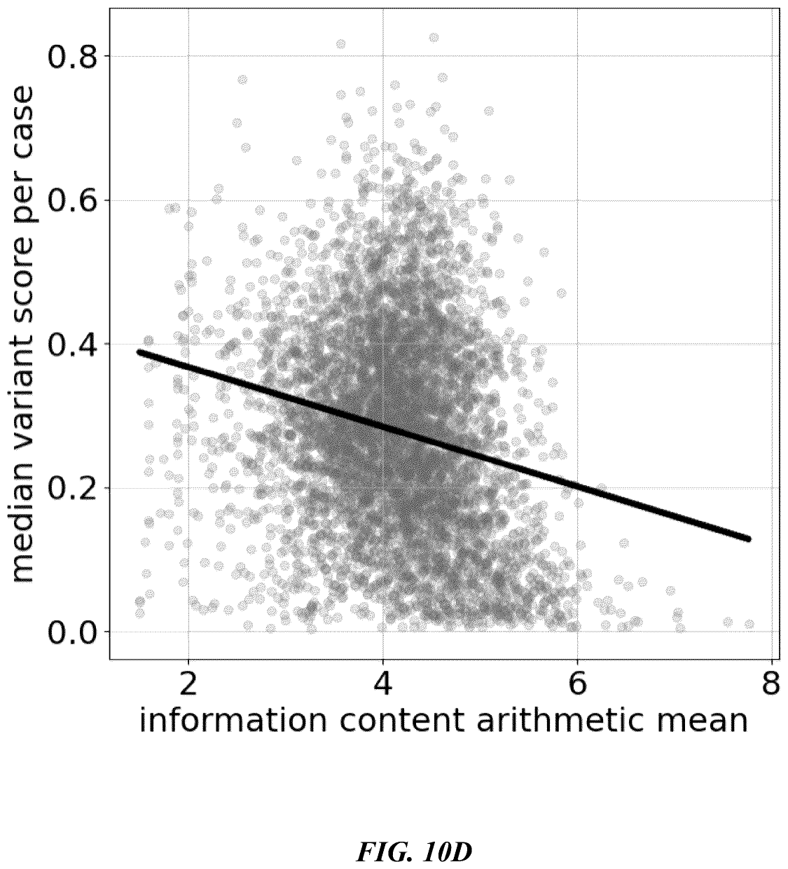

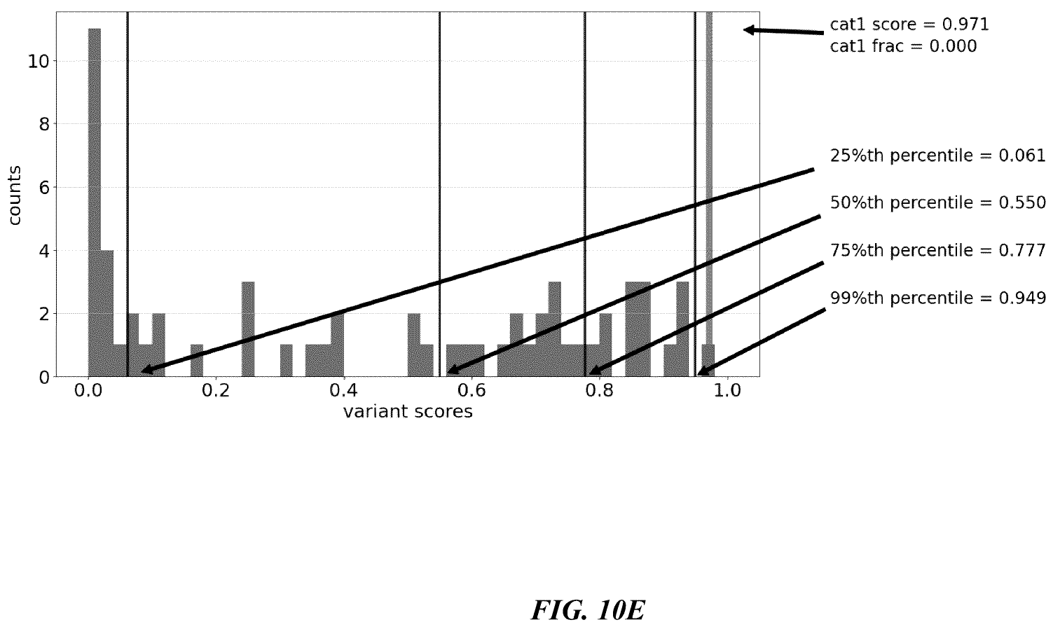

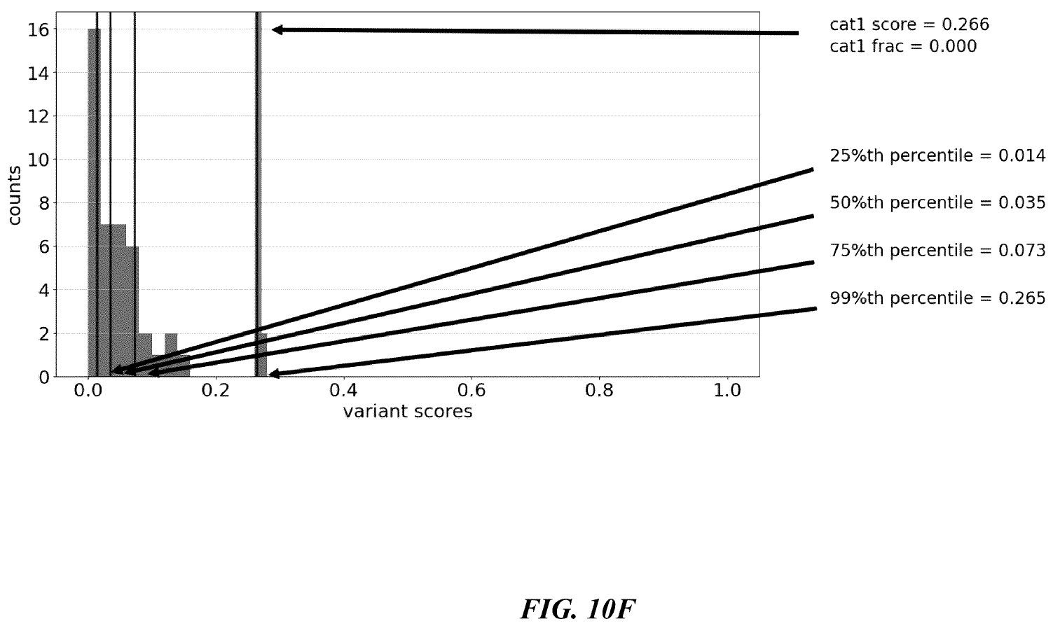

Various sensible filtering methods may be used. For example, filtering by absolute score comprises dropping/masking all case genes (variants) where the Multiscore value for that gene is less than a certain threshold value (e.g., “drop all variants in genes whose Multiscore value is lower than 0.50.”). As another example, filtering by score rank comprises dropping/masking all genes where the score (Multiscore value) for that gene is below the threshold set by percentile (e.g., “drop all variants in genes in the bottom 50% of scores for that case.”). Filtering by score rank was performed. Ranking of relevant items may be performed in Information Retrieval, such as defining relevant documents or web pages. See FIGS. 10A-10F for comparisons of rank and absolute score.

FIG. 10A shows the distribution of rank of the diagnostic gene for each case, described as a fractional value over the number of case genes. The rank is the percentile; a rank of 0.2 means the diagnostic variant was in the top 20% of scores of all pipeline genes. For example, if the diagnostic gene has the sixth highest score and there are 200 case genes, the rank for this gene is (6-1)/200=0.025. One was subtracted from the rank so that the top rank is defined to be 0.0. FIG. 10A shows that in most cases, Multiscore performs quite favorably; the diagnostic variant ranked in the top 10% of case genes 68% of the time.

FIG. 10B compares the distribution of ranks (blue) to the distribution of scores (orange) of all cases. Scores are plotted as distance (1.0-score) to allow direct comparison of the rank and score metrics. A log scale axis is often helpful in visualizing data separated by orders of magnitude. A log scale was used on the vertical axis to illustrate the long tail of cases with low performance. The score is shifted to the right compared to rank (worse performance) in almost all histogram bins.

FIG. 10C compares the score and rank of individual cases on a scatter plot. Most cases are sub-linear (below the green dotted line y=1−x) which shows that a case can often have a diagnostic gene with a low score while maintaining a favorable rank. This is because the distribution of scores for all case genes can shift, which may be caused by the specificity of the case terms.

FIG. 10D shows the mean information content (IC) of all terms versus the median Multiscore value of all case genes. The IC of a term i is defined as IC=−log(nᵢ/nₜₒₜ), where m is the number of genes annotated to term i in DiPR SDC1 cases and Mot is the total number of genes with an SDC1 diagnosis in DiPR. IC is a measure of specificity: the less frequently a term is used, the higher its semantic value. The best fit line (Pearson's R2=0.04) in FIG. 10D indicates that the usage of high IC terms in a case tends to result in fewer genes with strong phenotypic matches. FIGS. 10E-10F show two example cases where the distribution of scores has no effect on the rank of the diagnostic gene.

FIGS. 10A-10F show evaluation of absolute score (Multiscore value) versus rank of diagnostic gene FIG. 10A shows a histogram of fractional rank of diagnostic gene amongst all case genes. A rank of 0.0 means the diagnostic gene had the highest Multiscore value of all case genes (the most desirable result), and a rank of 1.0 means the diagnostic gene had the lowest Multiscore value. Height of bar is the number of cases with the given rank. FIG. 10B shows a histogram of diagnostic gene rank (blue) overlaid with histogram of diagnostic gene Multiscore value (orange) shown as distance or 1-Multiscore value to easily compare the different metrics. Vertical axis is shown as log scale of case count to elucidate the differences on the long tail of poor performance (high rank, low score). FIG. 10C shows a scatter plot of Multiscore value versus rank of diagnostic gene. Each point is a single case. Green dotted line represents y=x. FIG. 10D shows a scatter plot of mean IC of all case HPO terms versus median Multiscore value of all case. Each point is a single case. Line of best fit shown in black with R2=0.04. FIG. 10E shows a histogram of Multiscore values of all case genes depicting a case with many high scoring case genes. The diagnostic gene Multiscore value (cat1 score) is 0.971 which is the top ranked value (cat1 frac=0.0). FIG. 10F shows a histogram of Multiscore values of all case genes depicting a case with low scoring case genes. The diagnostic gene Multiscore value (cat1 score) is 0.266, however this is still the top ranked value (cat1 frac=0.0).

TABLE 11
Rank threshold compared to the false negative
(FN) rate. Results for 4,973 SDC1 cases

| rank | number of | |
|---|---|---|
| threshold | FNs | % FNs |
| 0.1 | 1513 | 30.8% |
| 0.2 | 777 | 15.8% |
| 0.3 | 424 | 8.6% |
| 0.4 | 267 | 5.4% |
| 0.5 | 164 | 3.3% |
| 0.6 | 112 | 2.3% |
| 0.7 | 63 | 1.3% |
| 0.8 | 36 | 0.7% |
| 0.9 | 19 | 0.4% |

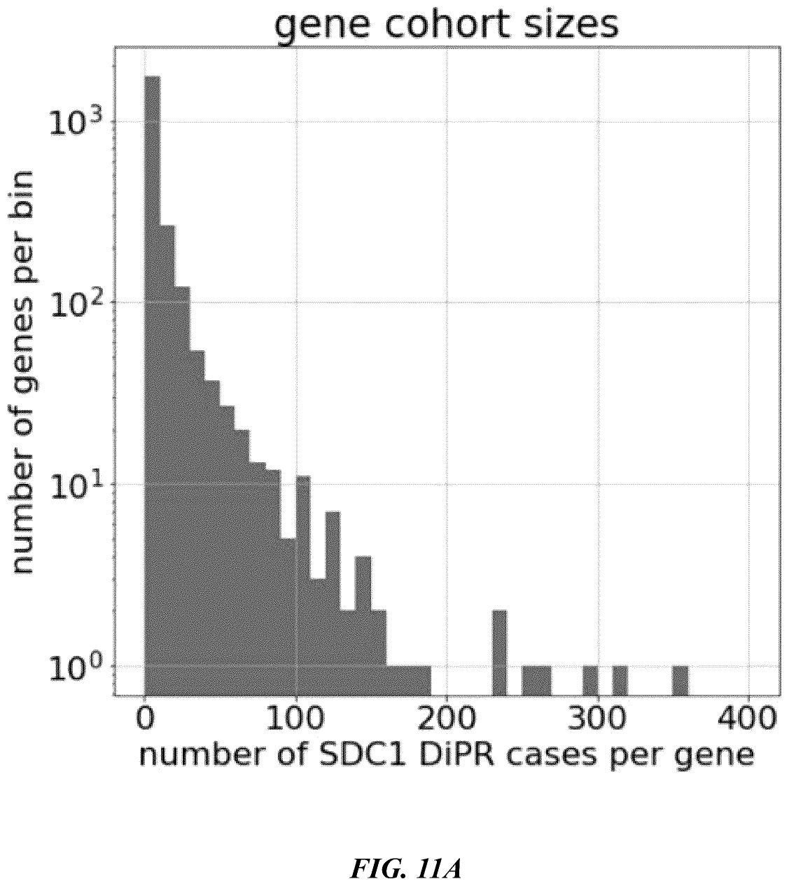

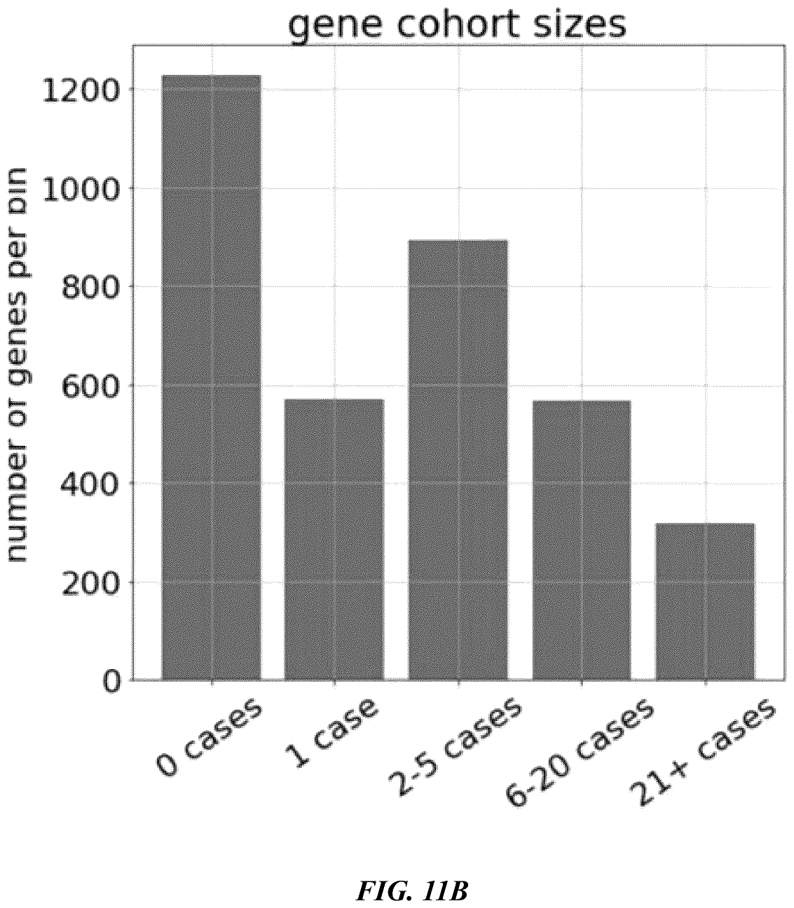

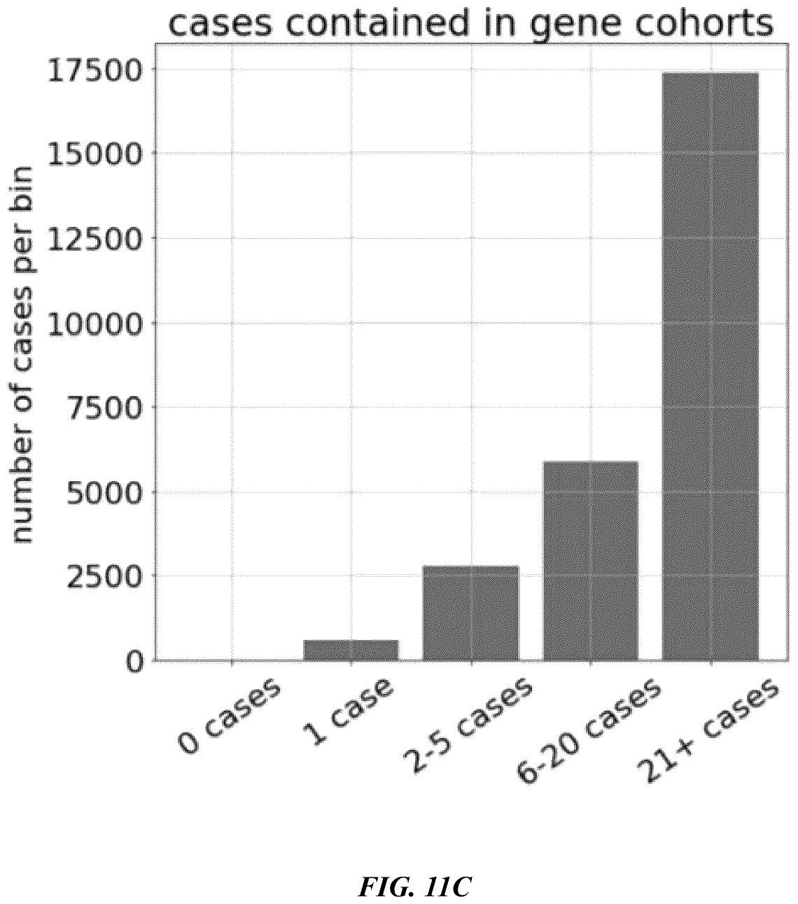

Having chosen to progress with a rank-based threshold, consider the rank thresholds for filtering and the FN rate that results from these thresholds. The percent FNs is the number of diagnostic genes that are hidden divided by the case count (4,973). Table 11 gives the FN rate for decile thresholds. Imagine the rank threshold as a vertical line moving across FIG. 11A; anything to the right of the line represents a FN case. Comparing Table 5 to FIG. 11A, there are 1,593 cases to the right of rank=0.1. As the imagined vertical line moves right across FIG. 11A and down Table 11, the threshold becomes more conservative, FNs are reduced, and the proportion of genes removed from analysis are reduced. In practical terms, this illustrates the delicate balance that must be struck during ramping up of testing throughput: Aggressive filtering leads to reducing the time spent on clinical review of pipeline variants while potentially leading to some loss in diagnosis rate.

Next, it was considered whether all case genes ought to be treated equally in the filtering process. At a given timepoint, there are 2,350 unique genes with SDC1 cases. In the analysis, all SDC1 cases closed before the month of a case contribute to the DiPR-based gene-phenotype annotations. There is a wide disparity of gene disease representation in DiPR; see FIG. 11A for details. A gene cohort was defined as the SDC1 cases with diagnostic variants on a given gene, and the size of the gene cohort is defined by the number of SDC1 cases in that cohort. FIG. 11A is a histogram of all gene cohort sizes represented with a log scale vertical axis. FIG. 11B is a bar chart of 5 bins showing the number of empty gene cohorts (0 cases) singleton gene cohorts (1 case), small gene cohorts (2-5 cases), medium gene cohorts (6-20 cases), and large gene cohorts (21 cases). Empty gene cohorts are genes with “validated” status in GeneMb but no SDC1 cases in DiPR. Over 62% of non-empty gene cohorts (1,451/2,350) have five or fewer cases. On the other hand, over 87% of SDC1 cases (23,238/26,586) are from gene cohorts with 6 or more cases per cohort. FIG. 11C shows a bar chart showing the number of cases represented by each of the above cohort size bins. Therefore, the genes are weighted towards smaller cohorts, but the cases are weighted towards larger cohorts.

FIGS. 11A-11C show gene cohort sizes for 2,350 unique genes across 26,586 Single Diagnosis Category 1 (SDC1) cases in DiPR. FIG. 11A shows a histogram of number of SDC1 DiPR cases per gene. Height of bar is the number of gene cohorts with the given bin size, represented on a log scale. Bin size=10 cases. FIG. 11B shows a bar chart of gene counts per cohort size, grouped into singleton (1 case), small cohort (2-5 cases), medium cohort (6-20), and large cohort (more than 21 cases). FIG. 11C shows the number of cases represented by the genes in the cohort sizes in FIG. 11B. These data indicate that most of the 2,350 genes with SDC1 cases are members of singleton or small gene cohorts, however, most of the SDC1 cases are members of large gene cohorts.

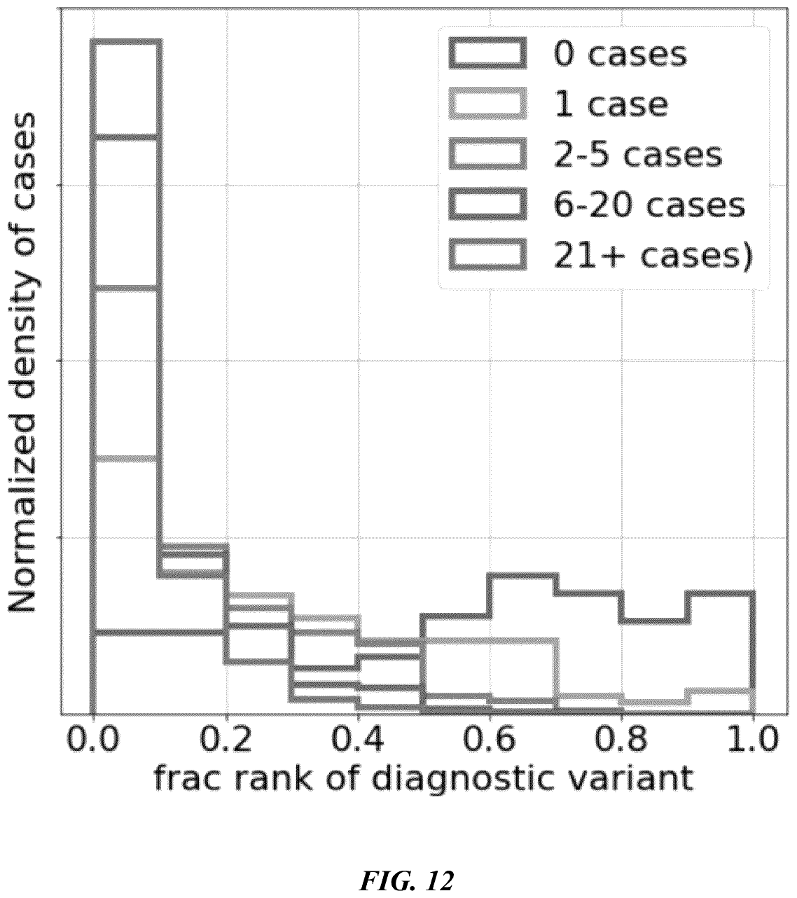

FIG. 12 shows case performance separated into gene cohort size of the diagnostic gene. In the analysis, gene cohort sizes were assigned per case and were determined by SDC1 cases that were closed before the month of the case being tested (see main text for details). Diagnostic genes tended to have higher score ranks (worse performance) when there was a low number of SDC1 cases in the DiPR data table. The histograms have been normalized such that the area underneath the histogram is equal for all five cohort sizes.

During the batched testing of cases in the POC analysis, DiPR was filtered for SDC1 cases closed in or before the previous month. Gene cohorts can grow from month to month as new diagnostic cases are added. All case genes were assigned gene cohort sizes based on the month they were analyzed. See FIG. 12 for the histogram analysis of case performance separated by gene cohort size of the diagnostic gene. The performance improves monotonically as the gene cohort size increases, with increasingly larger proportions of cases shifting left (lower ranks of diagnostic genes) on the horizontal axis. This indicates that the phenotypic spectrum becomes more complete as more cases are added to a gene cohort. Cases where the diagnostic gene had never been seen in DiPR (blue histogram, 0 cases) relied on only OMIM and literature data for scoring: because there were no gene-phenotype annotations for such genes, the Scorer values w2v_dipr, hrss_dipr, and jaccard_dipr were guaranteed to be 0.0 for these genes.

These data support establishing a threshold for gene cohort size in the downstream gene filtering process. In this mode, a gene must have a minimum number of SDC1 cases in DiPR before the associated pipeline search variant may be filtered/hidden. For example, rather than the threshold “filter out the bottom 50% of genes,” a solution may be “filter out the bottom 50% of genes if and only if the gene of the gene has been seen at least once in DiPR SDC1 cases.” This may improve the sensitivity at the expense of selectivity. The “random” way pipeline search genes are distributed over genes requires that most genes evaluated are on small or singleton cohorts (See FIG. 4). In fact, 34% (1,228/3,578) total disease genes have empty gene cohorts, and a similar proportion of 27.1% (159,752/589,465) pipeline search genes associated with disease genes from the POC analysis landed on empty gene cohorts. See Table 12 for the combined rank threshold and minimum gene cohort size as it affects the FN rate and average percentage of genes filtered. The genes filtered is about equal to (1-rank) when there is no minimum gene cohort size. As the minimum gene cohort size increases, however, the average percentage of genes filtered decreases quickly. This is because there are a limited number of medium and large sized genes cohorts.

TABLE 12
Rank threshold combined with minimum gene cohort size threshold
compared to False Negative (FN) rate. Shown are rank threshold
0.3, 0.4, 0.5, minimum gene cohort size 0, 1, 5, 10. The number
of FNs is the number of diagnostic genes that would be hidden
using these thresholds and the percentage FNs is the number
of FNs divided by the POC case count (3108). The average percentage
of genes filtered is the proportion of genes on disease genes
filtered per case averaged over all cases.

| | minimum | | | average % |
|---|---|---|---|---|
| rank | gene | Number of | | genes |
| threshold | cohort size | FNs | % FNs | filtered |
| 0.3 | 0 | 424 | 8.6% | 69.6% |
| 0.3 | 1 | 311 | 6.3% | 43.9% |
| 0.3 | 2 | 247 | 5.0% | 31.6% |
| 0.3 | 5 | 156 | 3.2% | 15.0% |
| 0.3 | 10 | 95 | 1.9% | 7.0% |
| 0.4 | 0 | 267 | 5.4% | 59.8% |
| 0.4 | 1 | 166 | 3.4% | 34.4% |
| 0.4 | 2 | 120 | 2.4% | 23.5% |
| 0.4 | 5 | 73 | 1.5% | 10.2% |
| 0.4 | 10 | 38 | 0.8% | 4.3% |
| 0.5 | 0 | 164 | 3.3% | 50.1% |
| 0.5 | 1 | 71 | 1.4% | 25.3% |
| 0.5 | 2 | 40 | 0.8% | 16.0% |
| 0.5 | 5 | 24 | 0.5% | 6.1% |
| 0.5 | 10 | 14 | 0.3% | 2.3% |

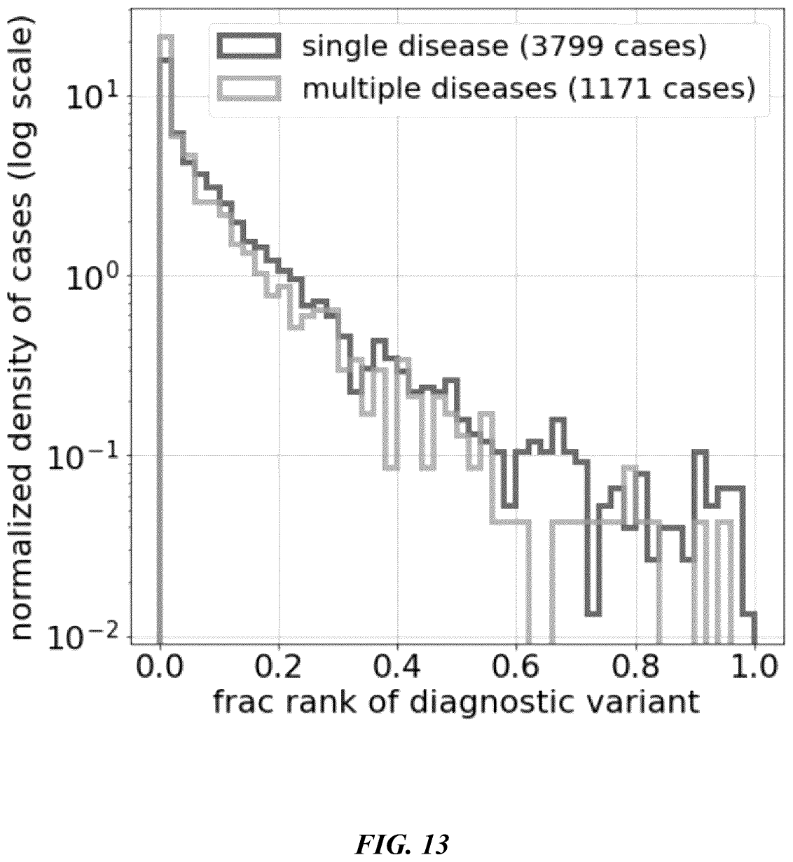

Further, there is an interest in pursuing disease-level phenotypic comparisons in Multiscore. When gene-level annotations are used, there may be concerns that the phenotypic information from several distinct diseases may be combined and affect scoring. Evaluation was performed on the performance of diagnostic variants on genes with a single disease and genes with multiple diseases. Disease entries included those in GeneMb with the status “validated” and the “is-mendelian” tag applied. See FIG. 13 for histograms of the diagnostic gene ranks for these two categories of genes. Diagnostic variants on genes with a single validated disease accounted for 3,799 cases, and diagnostic variants on genes with multiple validated diseases accounted for 1,171 cases. Qualitatively, the distribution of ranks of these two classes of gene were similar. There was a higher proportion of cases on multi-disease genes with a rank of 0.02 or better. There is no major concern related to scoring genes with more than one disease.

FIG. 13 shows case performance separated into genes with single diseases and genes with multiple diseases. Disease count was taken from GeneMb validated gene diseases. Blue: Genes with a single disease. Orange: genes with multiple diseases. Genes with multiple diseases were found to have a higher proportion of cases with rank less than 0.02. Vertical axis is visualized with log scale.

Some disease entries in GeneMb represent more than one disease in OMIM, however, this does not introduce complexities into our analysis. These lumped GeneMb disease entries represent a continuous spectrum of diseases, and based on case information, it is not always possible to say where in the spectrum a specific patient may fall. For example, “PNKP-related neurodegenerative disorder” https://genemb.genedx.com/diseases/GENEDX: 12909/is associated with three diseases in OMIM: microcephaly, seizures, and developmental delay (MCSZ); ataxia with oculomotor apraxia (AO4); Charcot-Marie-Tooth type 2 (CMT2). These diseases present within an age range that spans prenatally all the way through adulthood, with overlapping clinical features. When a patient is evaluated for this GeneDx disease, an overall clinical fit is determined in some cases, instead of defining a more specific match.

A set of thresholds and the False Negative created by those thresholds are examined. Multiscore serves as one arm variant filtering program in XA and other workflow platforms. A full validation of the filtering methods is performed before implementing any filtering thresholds in production, however, there is value in analyzing Multiscore-based thresholds only.

With Multiscore rank threshold=0.5, minimum gene cohort size=1, filtration of 25.1% of disease gene variants from the clinical analysis process. This leaves 71 FN cases, a rate of 1.4% of all cases.

General scientific modes of Multiscore were analyzed in some cases, rather than case-level analysis. Out of 71 FN cases, 27 had the phenotype was considered a strong fit for the diagnosed disease. The other 44 cases were categorized as, e.g., “general fit for NDD”, “nonspecific phenotype fit”, “low phenotype count”, “new disease, not in OMIM, low case count”.

In some cases, there were one or two highly specific phenotypes annotated to the patient that are considered diagnostic for the diagnosed disease. The list of DiPR- and literature-based gene-phenotype annotations can be quite long; see Table 13 for examples.

One possible solution to capture the frequency and specificity of HPO terms is to employ the Term Frequency inverse Document Frequency (TF-iDF) statistic. TF-iDF is a lumped term that captures frequency and specificity of, in this case, HPO terms across gene disease cohorts. TF-iDF values are maximal in the scenario of an HPO term that shows up on every case in a gene cohort and does not show up in any other gene cohort (100% frequency and 100% specificity).

The calculations of subscores may be weighted by the TF-iDF. Table 13 shows the TF-iDF scores of these diagnostic phenotypes in the DiPR and literature gene-phenotype annotation corpuses. The DiPR-based TF-iDF values in Table 7 were all significantly (greater than one standard deviation) above the average score across all 294,120 gene-phenotype annotations in the entire SDC1 exome cohort. Only one out of four literature TF-iDF scores were significantly above average, and one term was even missing from the literature data. This suggests that one may benefit from relying on retrospective case data.

TABLE 13

| | | Highly | | | | |
|---|---|---|---|---|---|---|
| | | specific | Number of | Number of | | |
| Gene | | phenotype | phenotypes | phenotypes | DiPR | Lit |
| Symbol | HGNC ID | present in case | in DiPR | in literature | TF-iDF | TF-iDF |
| G6PD | HGNC: 4057 | HP: 0410176 | 737 | 177 | 2.117 | Not |
| | | | | | | found! |
| CFTR | HGNC: 1884 | HP: 0004401 | 453 | 307 | 2.035 | 0.753 |
| MMACHC | HGNC: 24525 | HP: 0003223 | 158 | 144 | 1.175 | 0.590 |
| LMX1B | HGNC: 6654 | HP: 0002164 | 192 | 164 | 1.345 | 1.828 |
| AVERAGE | — | — | — | — | 0.701 | 0.576 |
| TF-iDF | | | | | | |

One further point is the limits of the HPO: there are not always HPO terms for important case phenotype features. For example, while many enzyme deficiencies are handled in the HPO, some diagnostic phenotypes like “3-methylcrotonyl coenzyme A carboxylase deficiency” are listed as an OMIM disease (www.omim.org/entry/210200) but not as a phenotype. One case had this former description in the medical notes with a diagnosis on the MCCC] HGNC: 6936 gene. Another example is “Lynch syndrome” in the unstructured text, which is consistent with reporting criteria for a cat1 diagnosis associated with MSH2 HGNC: 7325 but is not associated with an HPO term. Features like patient-to-patient doc2vec may be added, where these non-HPO descriptions are captured.

Further, the diagnostic variant(s) in all these False Negative cases given the set parameters for this analysis rank below the 50th percentile among all pipeline variants in a case, however, all of them are still present in Clinical Analysis mandatory searches. Multiscore may be used as an aid in evaluating phenotypic fit but may not replace the assessment of other variant properties examined within other pipelines searches, for instance, segregation evaluated in inheritance searches.

### Example 4: Multiscore Tool

Reporting in genomic testing may be phenotype driven; therefore, identifying variants in genes with a phenotypic fit may be fundamental. Current approaches may determine this measure by manual review by a clinical analyst and may be assisted by a pipeline search (e.g., “Phenotype” search) that assesses Human Phenotype Ontology (HPO) curated phenotypic data only. However, new disease genes, not yet curated in Online Mendelian Inheritance in Man (OMIM) have no phenotypic information and therefore may pose challenges.

Using systems and methods of the present disclosure, a multiscore laboratory workflow enhancement tool was developed to leverage machine learning to improve the efficiency and accuracy of trained clinical geneticists. The multiscore tool provides a framework for individualized geneticist assessment by ranking genetic variants based on the fit of a given gene with a subject's (e.g., patient's) reported clinical presentation. Multiscore utilizes information from OMIM-ORPHANET, peer-reviewed publications, and an internal database of patients that have been previously tested. This tool is used in conjunction with other data sources such as population databases, published literature, as well as the geneticist's clinical judgment, to aid the geneticist in decision making. This tool does not replace the human review, limit the genetic variants available for assessment by geneticists, nor limit the geneticist's ability to make an independent decision about the displayed variants.

An automated phenotype recognition algorithm, which may be referred to as Txt2hpo, was developed. It extracts phenotypes from text documents in the form of Human Phenotype Ontology (HPO) terms. This software algorithm leverages Python for natural language processing of biomedical text.

The Txt2hpo algorithm may break down input text into smaller manageable pieces for easier processing, which may be referred to as chunking.

The Txt2hpo algorithm may perform spell-checking, in which text tokens are spell-checked against a vocabulary of over 100,000 terms in the English language that includes biomedical words in addition to their frequency in the English language. The algorithm may be based on Peter Norvig's implementation of spell-checking (as described in norvig.com/spell-correct.html, which is incorporated by reference herein in its entirety). The algorithm considers up to two edits for terms that it doesn't immediately identify. Phenotype terms that are more complex or are simply missing from this vocabulary are incorporated manually into the vocabulary with each update to the Human Phenotype Ontology (HPO).

The Txt2hpo algorithm may perform tokenization and stemming, in which text is divided into individual tokens or words, and reduced to their base or root form (e.g., plural form of singular).

The Txt2hpo algorithm may perform identification of HPO terms. This operation leverage a pre-built search tree that the software creates based off HPO. The scanning of this tree is implemented using Python networkx, which may be used for network science-based applications.

The Txt2hpo algorithm may resolve conflicts. This operation leverages an internally built, patient-based doc2vec model, based on a neural network, that helps disambiguate conflicting terms. For example, if the free text includes the term “ASD”, this can match to either “Autism Spectrum Disorder” or “Atrial Septal Defect”. This doc2vec model leverages the surrounding context in the input text to determine which term the “ASD” abbreviation refers to.

The Txt2hpo algorithm may perform negation identification. This operation flags terms that are negated indicating the absence of the condition. This is implemented using a rules-based software, negspaCY, which identifies and processes negated terms within text, based on the NegEx algorithm. This software helps detect when text-extracted entities are negated by preceding or following negation phrases (e.g., “no”, “denied”).

The Txt2hpo algorithm leverages pre-built, rule-based methods and models, such as spaCy's language models and the HPO, which rely on established vocabularies and algorithms for processing. This doc2vec model was trained on clinical text from previously tested patients with suspected underlying genetic conditions that have been reported with causative variants.

A literature surveyor algorithm, which may be referred to as lit-surveyor, was developed that enables the automated downloading of all literature contained within the OA (Open Access) subset (PubMed, PubMed Central, NCBI GeneReviews) and subsequent aggregation with articles stored internally. This software algorithm leverages Python and multiple API endpoints. Multiscore leverages data and models produced by the lit-surveyor.

The lit-surveyor framework offers many tools to help mine biomedical text from publications and model them for use in downstream applications. For details, refer to the lit-surveyor design document. The lit-surveyor framework comprises downloading new articles, structuring of a corcpu, training a literature-based doc2vec model, scoring of gene-phenotype information, and retraction identification.

The lit-surveyor algorithm comprises downloading of all new articles, which leverages publicly available endpoints to download new literature that has been published. Abstracts and full-text articles are downloaded from PubMed and PubMed Central, respectively. The service also downloads files from NCBI's GeneReviews.

Further, the lit-surveyor algorithm comprises structuring of the corpus. The corpus refers to a large, structured collection of text data, represented as a massive data frame. Each entry in the corpus refers to a piece of individual text, extracted from downloaded articles and processed independently. Each entry is then subjected to txt2hpo for the identification of HPO terms, and associated with a PMID and a gene that is identified by leveraging the manually curated (by NCBI) gene2pubmed database (ftp://ftp.ncbi.nlm.nih.gov/gene/DATA/gene2pumbed.gz).

Further, the lit-surveyor algorithm comprises training the literature-based doc2vec model. All entries in the corpus that contain phenotype or gene information (˜0.3% of all articles) are subset into a smaller corpus that is used to train contextual models for downstream applications. This trained model is a neural network (doc2vec) that serves as a recommendation engine. A user can submit clinical text and gene information to the model and receive a similarity-based score dictating how contextually similar the submitted text is to literature that has been published for the gene of interest.

Further, the lit-surveyor algorithm comprises scoring of gene-phenotype information. Using the text2hpo identified phenotype terms (HPO) and the annotated gene information within the sub-setted corpus, the term-frequency inverse-document-frequency (TF-IDF) is calculated to score gene-phenotype associations (higher scores indicate a stronger association). These scores are then normalized to account for longer documents that may skew the scores due to over-representations between a set of genes and phenotype terms. Though this normalization technique has a lower bound of 0.4, the values are not strictly bound by an upper limit.

Further, the lit-surveyor algorithm comprises retraction identification. The PMIDs for all articles that have been flagged as retracted by NCBI are then compared to articles in an internal knowledgebase. If an article has been flagged and used for evidence in a case, then the clinical staff is notified, and the reference is removed from gene summaries. If the reference was used in variant interpretations, it is determined if an updated report would be needed.

Literature surveyor is trained using various data sources. For example, the literature-based doc2vec model is trained with articles from the PubMed, PubMed Central Full Text repositories, GeneReviews, and other clinical literature. PubMed comprises at least 35 million documents, PubMed Central comprises at least 7 million documents, and Gene Reviews comprises at least 856 chapters. Periodically, every literature abstract from PubMed and each full-text document from PubMed Central is processed by txt2hpo to identify publications that have human phenotypes mentioned. This doc2vec model is trained on the documents that contain HPO terms to develop a latent representation of the clinical literature on a periodic cadence.

Multiscore leverages a ML random forest (RF) model to learn associations between patient-gene similarity subscores and previously reported positive results to make a prediction on the association between a current patient and a gene associated with a variant call. Each subscore is a combination of a scoring algorithm and a data source. Three subscores leverage AI/ML, including word2vec algorithm plus a gene database data source, word2vec algorithm plus a literature surveyor data source, and a doc2vec algorithm plus a literature surveyor data source. Word2vec and doc2vec are natural language processing semantic representation algorithms that are trained.

Multiscore comprises an RF model that was trained on exome sequencing(ES) and genome sequencing (GS) case. Certain cases are removed, including 1) healthy probands, 2) prenatal probands, and 3) multiple reports per case. The RF model was trained to learn the relationship between the numerical feature scores and the positive/not positive status of a gene in a retrospective internal patient case.

### Example 5: Multiscore Algorithm Shows High Sensitivity for the Molecular Detection Of Mendelian Disorders in Nearly 10,000 Exomes and Genomes

Using methods and systems of the present disclosure, a multiscore algorithm was developed that demonstrated high sensitivity for the molecular detection of Mendelian disorders in nearly 10,000 exomes and genomes.

### Background

A challenge for clinical exome and genome sequencing analysis may be identifying similarities between cases being tested and gene-disease associations curated in various databases or reported in the literature. Multiscore, a gene prioritization tool, was developed to facilitate gene-level predictions of phenotypic fit.

### Methods

Multiscore combines data inputs and algorithms to generate similarity subscores that feed a random forest (RF) classifier trained to predict the probability of association between the patient's clinical features and the gene. The reference gene-phenotype knowledge is extracted from: 1) OMIM, 2) patient descriptions in the literature, and 3) GeneDx (GDx) clinical data. For example, 9,989 exome and genome cases were utilized to assess performance of the tool in combination with genotype filtering and describe the strategy for implementation in a clinical setting.

### Results

Genotype filters rendered an average of 173 genes with variants requiring clinical review. Multiscore prioritized the reported positive gene with a median rank of 3 and mean rank of 6.35. The average recall (sensitivity) of Multiscore was 33% in the top 1, 69% in the top 5, 83% in the top 10, and 93% in the top 20 ranked genes. Multiscore performance was not impacted when multiple diseases were associated with a gene and was able to handle non-exact HPO term matches allowing the use of real-world clinical data. 74 genes lacking OMIM entries were prioritized using only the GDx and literature datasets, contributing to the diagnosis of 257 cases associated with ultra-rare disorders.

By incorporating curated datasets of gene-phenotype associations and real-world clinical data, Multiscore removes the bias against those individuals with milder disease, disorders with variable expressivity, and newly described disease genes with phenotypes that are yet to be fully defined. Multiscore allows the phenotype review to prioritize the most relevant variants, increasing case throughput and broadening access to diagnoses for patients.

### Introduction

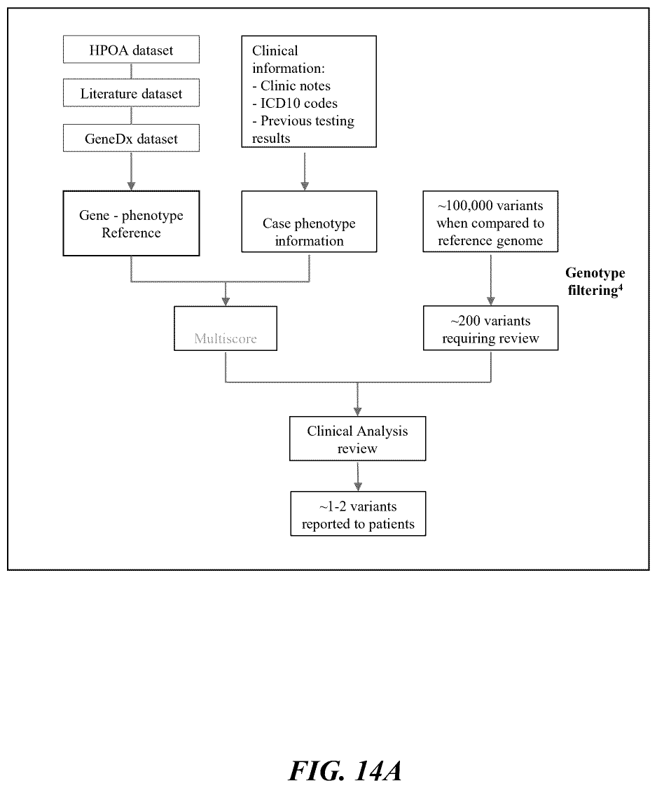

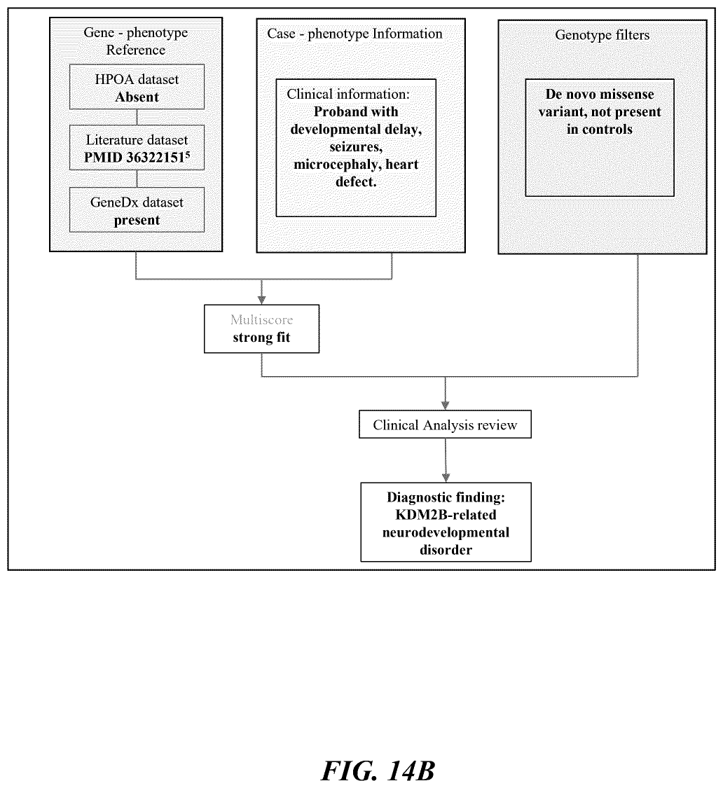

One of the foremost challenges of clinical exome and genome sequencing (ES/GS) is efficiently filtering down the thousands of variants identified in a patient's DNA to a relevant few that could be contributing to an individual's clinical symptoms (i.e., their phenotype). This may be accomplished by combining analysis of variant pathogenicity informed by multiple computational algorithms, frequency in the general population, and clinical review of the phenotype, which in turn requires a deep understanding of gene-disease relations (FIG. 14). Clinical information can be translated into a standard vocabulary, the Human Phenotype Ontology (HPO), then assessment of multiple databases of gene-phenotype associations (GPAs) such as the Online Mendelian Inheritance in Man (OMIM), Orphanet and GeneReviews, alone or in combination with literature reviews, may be needed. This clinical review remains a difficult task, complicated by long GPA sets, identification of relevant phenotypes in the literature and patient records, questions around non-exact phenotype matching, and consideration of disease-versus gene-level information.

Automated tools to assess phenotypic overlap rely on published curated datasets to highlight genes or variants likely to be associated with a particular term using similarity metrics, statistical models, or trained machine learning (ML) models. Some tools use only the patient phenotypes but supplement the reference GPAs with protein, gene, or pathway interactions. Other tools prioritize variants detected in a patient case and consider the phenotype similarity alongside variant frequency and other predictors of pathogenicity. Newer tools like AMELIE incorporate a literature search to construct associations between gene, variants, and phenotypes, thus addressing a major limitation of the previous models at the potential expense of deprioritizing unpublished variants. Few tools rely on case phenotypes or gene phenotypes alone and only recently have there been attempts at using electronic health records (EHR) to derive phenotype data. Even when using real patient data, cohorts are typically part of gene discovery programs or cases from the literature, which include deep phenotyping by genetic specialists and do not always translate to the clinical information provided to genomics testing laboratories performing case analysis.

For efficient abstraction of large amounts of clinical information and quantitative integration of these data into case analysis, Multiscore was developed, a gene prioritization tool that uses an ML model to combine multiple measures of case-gene phenotypic similarity. Multiscore is informed by a large dataset of well-characterized individuals in addition to external data sources. This artificial intelligence tool highlights phenotypes that may be missed by direct comparison with published curated datasets and allows the phenotype review process to focus on the variants in the most relevant genes, increasing diagnostic yield while reducing analysis time per case.

### Methods

Three sources of data were utilized, for example: (1) disease-phenotype annotations extracted from OMIM (HPOA dataset), (2) patient descriptions obtained from the relevant published literature (Lit dataset), and (3) a structured clinical dataset describing probands with a positive finding identified by exome or genome sequencing at GeneDx (GDx dataset). Clinical information for the GDx dataset was obtained from clinical notes submitted with the sample, ICD10 codes, and relevant client communications. A positive finding was defined as the presence of variant(s) matching the accepted mode of inheritance in a disease gene with good phenotypic fit and classified as pathogenic or likely pathogenic according to American College of Medicine classification criteria. Cases with dual molecular positive findings and cases where the positive finding was a multi-gene copy number variant were excluded from the GDx dataset, model training, and model testing.

### Literature Surveyor

There are over 100,000 new potentially relevant publications in PubMed every month. Literature Surveyor is a toolkit designed for data mining and managing scientific literature describing gene-phenotype relationships. This software enables the automated downloading and formatting of publicly available abstracts and full-text documents to construct a comprehensive corpus. Literature Surveyor leverages natural language processing (NLP) methodologies to identify associations between genes and phenotypes. Literature Surveyor performs three main functions: 1) downloading and processing all articles in the corpus, 2) calculating gene-phenotype annotations found in the corpus, and 3) training a doc2vec model to learn and generate embeddings from gene and phenotype-rich articles within the corpus (Supplementary methods). The sources of documents comprising the corpus include MEDLINE/Pubmed, PMC Articles from NCBI, and GeneReviews.

txt2hpo

For example, txt2hpo was utilized, an in-house developed automated phenotype recognition tool that leverages NLP to extract phenotypes from the Lit and the GDx datasets in the form of HPO terms.

### Knowledge Reference

A dynamic knowledge base of phenotype information, variant classifications, and gene-disease associations informs the reporting strategy. Some disease entries in the GeneDx knowledge reference represent more than one disease in OMIM. Diseases were consolidated or “lumped”. Disorders that shared a common inheritance pattern, mechanism of disease, and phenotypic features within and/or between families segregating the same variant(s), were reviewed by curators and lumped. These lumped disease entries represent a continuous spectrum of diseases, and based on case information, it is not always possible to say where in the spectrum a specific patient may fall. For example, the CEP290 gene is associated with five diseases in OMIM: Bardet-Biedl syndrome (MIM 615991), Joubert syndrome (MIM 610188), Leber congenital amaurosis (MIM 611755), Meckel syndrome (MIM 611134), and Senior-Loken syndrome (MIM 610189). These diseases share overlapping clinical features, inheritance, and mechanism of disease and thus were lumped under the CEP290-related ciliopathy umbrella. When lumping was not possible after curator review, the entries were kept separate.

### Data Processing

All three data sources were processed to produce GPAs which comprise all phenotypes associated with each gene. GDx dataset GPAs were obtained from the list of unique phenotypes across all the patients with single gene positive findings in that gene in the dataset. Literature dataset GPAs were collected from phenotypes extracted from publications using the gene2pubmed database. HPOA dataset GPAs were collected from the disease-level OMIM phenotype annotations. Prioritization scoring also depended on a word2vec (arxiv.org/abs/1301.3781) model and doc2vec (arxiv.org/abs/1405.4053) model trained in-house.

### Model Design

Multiscore considered the phenotypic profile of a patient against a list of genes. For each gene, data inputs and algorithms were mixed in combination to generate eight patient-gene similarity subscores (Table 17) used as features for the ML random forest (RF) classifier (arxiv.org/abs/1603.02754), which yielded a probability of association between a patient's clinical description and a gene. Multiscore allows non-exact HPO term matching by using: 1) doc2vec to convert patient medical text to vector embeddings and compare the embedded text to sentences from publications in the Literature Surveyor corpus, 2) Hybrid Relative Semantic Similarity (HRSS) to assess the distance between HPO terms in the HPO graph and their information content, and 3) word2vec to interpret the local context of HPO terms in literature. Exact HPO term matching between the case and the gene knowledge dataset is evaluated using Jaccard similarity.

TABLE 17
Description of Scorers in Multiscore. Scorers are
a combination of data source and scoring algorithm.

| | | Scoring | |
|---|---|---|---|
| Scorer Name | Data Source | Algorithm | Note |
| HRSS HPOA | HPOA-OMIM Disease | HRSS | Case HPO terms are compared |
| | Phenotype | | to all disease HPO terms in |
| | annotation file | | HPOA-OMIM, and the highest |
| | | | disease score for each gene |
| | | | is used. |
| HRSS GDx | GDx GPA file | HRSS | Case HPO terms are compared |
| | | | to all gene HPO terms from |
| | | | positive cases with a single |
| | | | diagnostic gene in the GDx |
| | | | dataset. |
| HRSS lit | Literature Surveyor | HRSS | Case HPO terms are compared |
| | GPA file | | to all gene HPO terms found |
| | | | in literature mining. |
| Jaccard GDx | GDx GPA file | Jaccard | Case HPO terms are compared |
| | | similarity | to all gene HPO terms from |
| | | | positive cases with a single |
| | | | diagnostic gene in the GDx |
| | | | dataset. |
| Jaccard lit | Literature Surveyor | Jaccard | Case HPO terms are compared |
| | GPA file | similarity | to all gene HPO terms found |
| | | | in literature mining. |
| w2v GDx | GDx GPA file, phenopy | word2vec | Case HPO terms are compared |
| | word2vec model file | | to all gene HPO terms from |
| | | | positive cases with a single |
| | | | diagnostic gene in the GDx |
| | | | dataset using HPO word |
| | | | embedding. |
| w2v lit | Literature Surveyor GPA | word2vec | Case HPO terms are compared |
| | file, phenopy word2vec | | to all gene HPO terms found |
| | model file | | in literature mining using |
| | | | HPO word embedding. |
| doc2vec | Literature surveyor | doc2vec | Embedded case text is compared |
| | doc2vec model file | | to embeddings of all documents; |
| | | | top score for each gene is the |
| | | | doc2vec score. |

The classifier did not learn information related to phenotypes or Mendelian disease; rather, it learned the relationship between the combinations of feature subscores that paired with a positive finding in a patient case. By training a classifier model on this combination of subscores from various datasets and algorithms, Multiscore learns to identify a strong signal of association between patient and gene from an ensemble of weaker signals.

The impact of incorporating the GDx dataset was evaluated by looking at the number of individuals included to create the gene reference information and the term frequency-inverse document frequency (TF-iDF) of all the HPO terms in each gene group. TF-iDF measures term importance within the cases associated with one gene, combining the frequency of a term within those cases with the presence of that term within all genes in the GPA corpus.

### Model Training and Testing

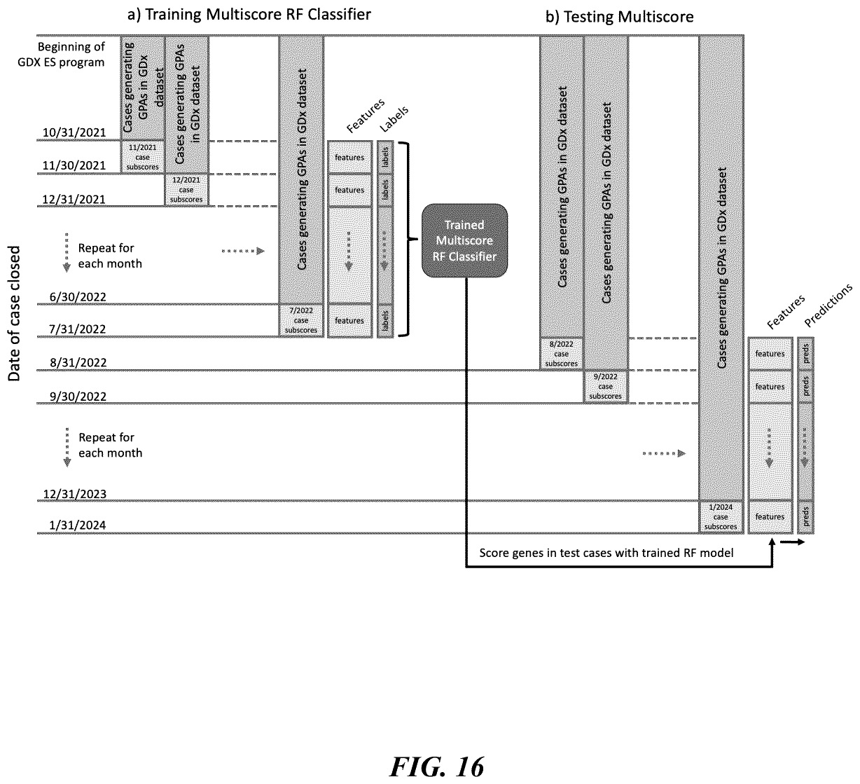

Training and testing were devised as a time-series experiment: the annotation data (GDx, HPOA, and Lit) and the subscores for training and testing cases were collected in monthly batches (FIG. 16). The testing case dataset was separate and distinct from the training case dataset. The training dataset comprised 3,654 positive cases and the testing dataset comprised 9,989 positive cases (Table 18).

TABLE 18
Characteristics of the GDx dataset used
for training and testing Multiscore.

| | Multiscore | Multiscore |
|---|---|---|
| | training | testing |
| | data | data |
| Number of cases | 3,654 | 9,989 |
| Age at testing (mean in years, SD) | 9.66, 11.3 | 9.62, 11.5 |
| Sex assigned at birth | | |
| Male (%) | 53.7% | 54.4% |
| Female (%) | 46.3% | 45.5% |
| Unknown (%) | 0.0% | 0.04% |
| Genetic ancestry prediction* | | |
| EUR (%) | 51.60% | 49.10% |
| AMR (%) | 24.60% | 25.80% |
| AFR (%) | 11.10% | 12.50% |
| MDE (%) | 6.94% | 6.87% |
| SAS (%) | 3.07% | 3.13% |
| EAS (%) | 2.69% | 2.54% |
| HPO branches represented per case (mean, SD) | | |
| abnormal cellular phenotype | 3.1% | 2.2% |
| abnormality of blood and blood-forming tissues | 15.0% | 16.6% |
| abnormality of head or neck | 70.6% | 67.4% |
| abnormality of limbs | 39.3% | 37.0% |
| abnormality of metabolism/homeostasis | 28.8% | 29.6% |
| abnormality of prenatal development or birth | 19.5% | 20.8% |
| abnormality of the breast | 4.30% | 3.6% |
| abnormality of the cardiovascular system | 38.6% | 39.7% |
| abnormality of the digestive system | 50.9% | 49.4% |
| abnormality of the ear | 33.3% | 32.9% |
| abnormality of the endocrine system | 13.0% | 12.3% |
| abnormality of the eye | 46.7% | 45.3% |
| abnormality of the genitourinary system | 26.0% | 26.1% |
| abnormality of the immune system | 31.4% | 32.1% |
| abnormality of the integument | 45.8% | 45.7% |
| abnormality of the musculoskeletal system | 79.1% | 77.2% |
| abnormality of the nervous system | 91.5% | 91.8% |
| abnormality of the respiratory system | 33.3% | 35.6% |
| abnormality of the thoracic cavity | 0.1% | 0.1% |
| abnormality of the voice | 3.9% | 3.3% |
| constitutional symptom | 19.2% | 18.0% |
| growth abnormality | 42.0% | 41.5% |
| neoplasm | 5.8% | 5.9% |

EUR = European, AMR = American admixed, AFR = African, MDE = Middle Eastern, SAS = Southeast Asian, EAS = East Asian.
HPO branch information shows the percentage of patients in each data set where the patient has one or more phenotypes in each branch.
*Genetic Ancestry Prediction was performed using Principal Component Analysis of the sample against a reference sample set from 1000 Genomes Project plus Middle Eastern patients from GeneDx. For details, see Lake et al. Table S3 (PMID 28777931).

FIG. 16 shows splitting retrospective training case set into gene-phenotype annotations, training data, and testing data. The vertical axis reflects the date of cases closed, which acts as a filter and grouping mechanism for case data. For each month of cases, separated by the date clinical genetic testing was completed, the eight subscores were generated based on the internal and external knowledge available on the last day of the previous month. The GDx and Lit GPAs and the Lit-Surveyor doc2vec model were updated for each monthly batch. The word2vec model trained internally and published in the GeneDx phenotype tool from phenopy was used for all batches. The HPOA release from Sep. 1, 2023 was used for scoring all batches. A key component is that the RF classifier model is trained once (blue box), but the data annotations that feed the subscores (GPAs and doc2vec model) are refreshed each month. This is possible because the RF classifier model only needed to learn the relationship between the combinations of feature subscores that paired with a positive finding in a patient case, as opposed to learning relationships between diseases and phenotypes.

Building training data for the Multiscore RF classifier. Green: GDx dataset GPAs (input to HRSS GDx, w2v GDx, Jaccard GDx subscores) built from single gene positive cases closed on the last day before the respective testing month. Yellow: Cases closed during the training month. Subscores were generated for the genes identified in genotype-filtering pipelines aka “pipeline genes” for the training cases. The subscores were generated in monthly batches from November 2021 to July 2022: a total of 3,654 cases. Orange: Labels for the pipeline genes in training cases (1 for genes reported positive, 0 for all other genes). The RF model is trained on these features and labels. During training, the negative labels were randomly downsampled to a negative: positive ratio of 5:1. The classifier model built during training made the predictions to give the final Multiscore probability during testing. The Multiscore probability was a probability of the association between a patient's clinical features and a gene.

Testing the machine learning classifier in a time-series analysis. Light green: GDx GPAs were built from cases closed before the current test month (see part a). Note that GPAs training cases were folded into the annotation data. Yellow: Cases closed during the testing month were used to test model performance. Subscores were generated for the genes identified in the pipeline genes for the training cases. The subscores are generated in monthly batches for a total of 9,989 cases. Orange: prediction values from the RF classifier model (the Multiscore probability; a probability of association between a patient's clinical features and a gene) based on the case feature subscores. The pipeline genes are ranked by the Multiscore and the rank of the reported positive gene is saved.

All genes with variants passing the genotype filters in a case were scored. The genes were ranked by the Multiscore probability and by the values of the eight subscores to compare the performance of Multiscore to each of the independent subscores.

The “recall at k” (or “sensitivity at k”) was utilized to measure how well Multiscore prioritized the positive finding gene identified by standard clinical analysis. The recall at k for a case describes whether the positive finding was ranked in the top k genes tested. The average over groups of cases was measured to give the average recall at k. In this analysis, where cases were tested with one relevant gene per case, average recall at k is equivalent to the fraction of patient cases where the reported positive finding gene ranked in the top k genes by Multiscore.

### Benchmarking

Analysis was performed of the same 9,989 cases using two other phenotype-only prioritization tools: Phrank using the GDx GPA knowledgebase, Phrank using the HPOA GPA knowledgebase and LIRICAL. Phrank was implemented in GORPipe. For Phrank GDx, the GDx knowledgebase was used to test cases in monthly batches as in Multiscore. For Phrank HPOA and LIRICAL, the HPOA release from Sep. 1, 2023 was used for scoring all batches. For LIRICAL, ORPHANET GPAs from the Sep. 1, 2023 release were included to supplement the OMIM entries. LIRICAL v2.0.2 was used to score cases using yaml files built for each case. LIRICAL was not run on 4 fetal cases with undetermined sex in the clinical information.

### Multiscore Implementation in a Clinical Setting

Genotype analysis filters focused on variant level indicators of pathogenicity and included: (1) segregation using joint calling with the parental genotypes, when available (i.e., de novo, compound heterozygous status), (2) variants reported in HGMD, ClinVar, or in an internal knowledge base, and (3) rare unclassified variants predicted to impact protein function. Variants with allele frequency >0.01 in gnomAD were excluded. These filters create a pool of variants for clinical review. Multiscore was not used to filter variants; instead, gene ranks of the Multiscore RF probabilities were displayed as an additional tool to inform that review. Since Multiscore assessed the phenotypic fit of genes, all variants in a gene are assigned the same score (i.e., compound heterozygous).

Multiscore Consistently Prioritized Genes with Positive Findings in 9,989 Retrospectively Analyzed Cases.

The average number of genes with variants requiring review in this analysis was 173 per case (90.1 for trios and 307 for non-trios) (Table 14). Multiscore prioritized the reported positive gene with a median rank of 3 and mean rank of 6.35 (standard deviation 10.2). Multiscore prioritized the positive finding in the top 1 gene in 33.1% of cases, in the top 3 genes in 57.1% of cases, in the top 5 genes in 69.1% of cases, in the top 10 genes in 83.5% of the cases, and in the top 20 genes in 93% of the 9,989 cases in the testing dataset. The recall performance was lower for non-trio cases which had less informed genotype filtering due to incomplete segregation information, but Multiscore still prioritized the positive finding in the top 10 genes in 76.4% of cases.

TABLE 14
Multiscore performance for all cases, trio cases, and non-trio cases.
Average recall at k, median rank, mean rank, and rank standard deviation
all refer to prioritization of the positive reported gene.

| | all cases | trios | non-trios |
|---|---|---|---|
| Cases (n) | 9,989 | 6,190 | 3,799 |
| Average recall at k | | | |
| 1 | 0.331 | 0.363 | 0.278 |
| 2 | 0.479 | 0.528 | 0.400 |
| 3 | 0.572 | 0.626 | 0.483 |
| 5 | 0.691 | 0.745 | 0.604 |
| 10 | 0.835 | 0.879 | 0.764 |
| 15 | 0.898 | 0.932 | 0.843 |
| 20 | 0.933 | 0.962 | 0.887 |
| Median rank | 3 | 2 | 4 |
| Mean rank | 6.35 | 4.99 | 8.57 |
| Rank standard | 10.2 | 7.35 | 13.42 |
| deviation | | | |
| Mean number of | 173 | 90.1 | 307 |
| genes per case | | | |
| Median number of | 108 | 85 | 284 |
| genes per case | | | |

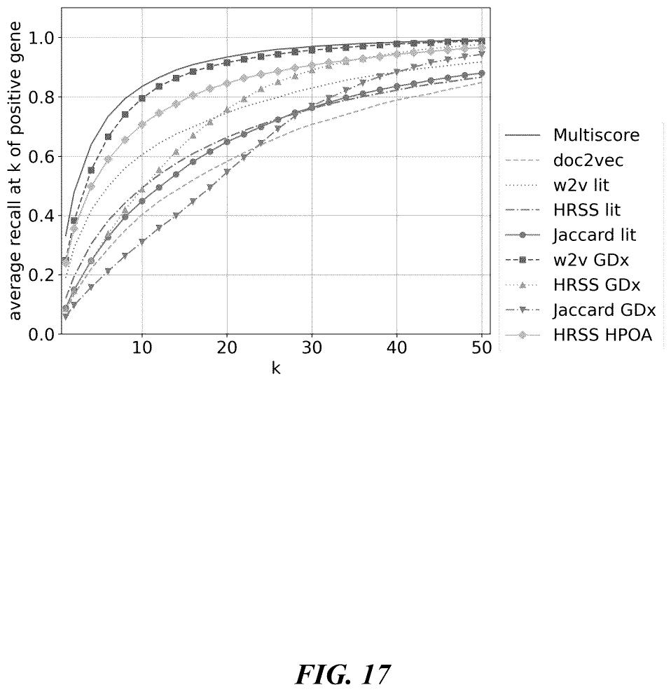

The performance of Multiscore was compared to rank the positive gene against each individual subscore for k=1 through k=50 (Table 19, FIG. 17). The average recall at k was always highest for Multiscore, followed by word2vec GDx, then by HRSS HPOA, until k=34, where the recall for HRSS GDx surpasses HRSS HPOA.

TABLE 19
Average recall at k of the reported positive gene for Multiscore and the
subscore features. Best average recall at each k value in bold.table

| | | | w2v | HRSS | Jaccard | w2v | HRSS | Jaccard | HRSS |
|---|---|---|---|---|---|---|---|---|---|
| k | Multiscore | doc2vec | lit | lit | lit | GDx | GDx | GDx | HPOA |
| 1 | 0.331 | 0.079 | 0.188 | 0.120 | 0.088 | 0.248 | 0.085 | 0.058 | 0.239 |
| 2 | 0.479 | 0.134 | 0.291 | 0.194 | 0.149 | 0.383 | 0.146 | 0.097 | 0.355 |
| 3 | 0.572 | 0.179 | 0.363 | 0.256 | 0.200 | 0.477 | 0.200 | 0.129 | 0.436 |
| 5 | 0.691 | 0.256 | 0.461 | 0.342 | 0.289 | 0.616 | 0.296 | 0.186 | 0.548 |
| 10 | 0.835 | 0.402 | 0.605 | 0.494 | 0.447 | 0.795 | 0.489 | 0.310 | 0.707 |
| 15 | 0.898 | 0.502 | 0.687 | 0.593 | 0.561 | 0.876 | 0.643 | 0.423 | 0.792 |
| 20 | 0.933 | 0.582 | 0.746 | 0.664 | 0.648 | 0.915 | 0.758 | 0.546 | 0.845 |
| 30 | 0.969 | 0.707 | 0.830 | 0.760 | 0.763 | 0.957 | 0.890 | 0.769 | 0.906 |
| 40 | 0.984 | 0.789 | 0.883 | 0.822 | 0.835 | 0.978 | 0.948 | 0.883 | 0.943 |
| 50 | 0.990 | 0.848 | 0.917 | 0.866 | 0.879 | 0.987 | 0.976 | 0.943 | 0.965 |

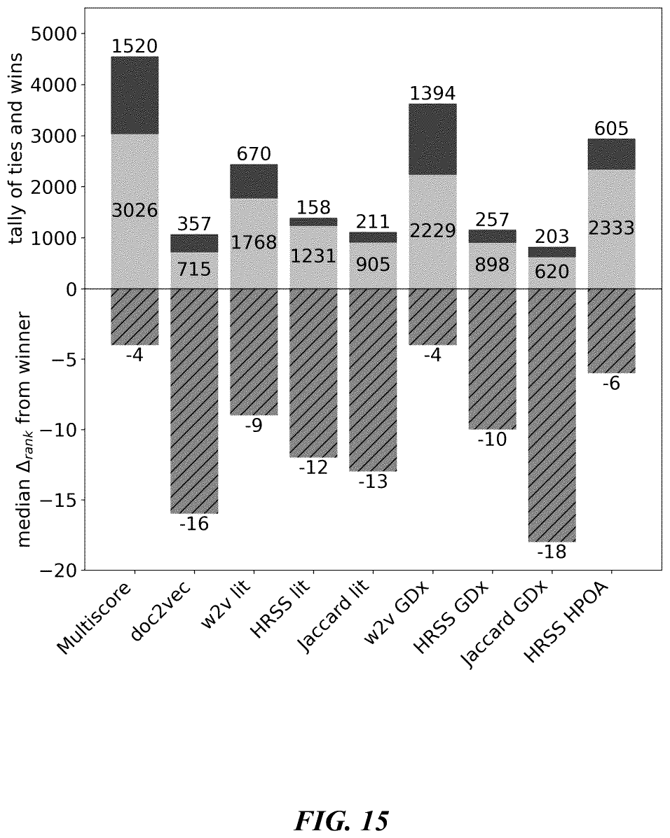

The overall wins tally of Multiscore and each subscore is shown in the top bar chart of FIG. 15. Multiscore gave the highest ranking for the positive finding compared to the subscores in 1,520 cases and tied for highest in an additional 3,026 cases, for a total of 4,546/9,989 (45.9%) cases tested, followed by word2vec GDx, which was the highest-ranking score in 1,394 cases and tied for highest in 2,229 cases, for a total of 3,623/9,989 (36.6%) cases. For cases where a score was not the highest rank (or tied for highest), tracking was performed for Dᵣₐₙₖ, the difference between that score's rank and the winning score's rank. The bottom bar chart of FIG. 15 shows the median Dᵣₐₙₖ for each score; the smallest median Dᵣₐₙₖ was −4 for both Multiscore and word2vec GDx.

As the GDx positive case count of the diagnostic gene (cc) increased, so did the predictive ability of Multiscore (Table 15). Genes with cc=0 have not been reported as positive results previously, thus there is no phenotypic information in the internal knowledge dataset, and the Multiscore model relies on literature and OMIM knowledge datasets.

TABLE 15
Average recall at k of Multiscore for all cases based on
GDx case count of the positive gene (cc). “n cases”
refers to the number of cases with the relevant cc values.

| k | all cases | cc = 0 | cc = 1 | cc = 2-5 | cc = 6-10 | cc³ 11 |
|---|---|---|---|---|---|---|
| 1 | 0.331 | 0.023 | 0.080 | 0.146 | 0.211 | 0.384 |
| 2 | 0.479 | 0.046 | 0.133 | 0.250 | 0.333 | 0.548 |
| 3 | 0.572 | 0.083 | 0.189 | 0.308 | 0.418 | 0.648 |
| 4 | 0.639 | 0.111 | 0.225 | 0.364 | 0.489 | 0.719 |
| 5 | 0.691 | 0.134 | 0.261 | 0.424 | 0.552 | 0.769 |
| 10 | 0.835 | 0.217 | 0.462 | 0.614 | 0.725 | 0.904 |
| 15 | 0.898 | 0.304 | 0.574 | 0.758 | 0.832 | 0.951 |
| 20 | 0.933 | 0.406 | 0.671 | 0.839 | 0.906 | 0.971 |
| n cases | 9,989 | 217 | 249 | 948 | 881 | 7,694 |

Multiscore can Prioritize Genes that are not Curated in Public Reference Databases

The Multiscore testing set included 74 genes without HPOA information, impacting 257 cases (Table 20). In 231/257 (90%) cases, the positive gene had been previously seen in the GDx dataset (61 genes). Data in the internal and literature datasets brought the Multiscore phenotypic rank within the 20 top genes in 48.2% of the cases (recall of 0.482 at k=20) allowing prioritization of newer and ultra rare disease associations. One of these genes, KDM2B, did not have a curated entry in OMIM, Orphanet, or GeneReviews, it had however one large publication reporting the phenotypic spectrum in 27 individuals and was included in the GeneDx knowledge reference; Multiscore ranked the KDM2B gene within the top 20 genes in 4/8 cases (FIG. 14B).

TABLE 20
Average recall at k of Multiscore for cases with no
HPOA based on internal case count of the positive gene
for the given case (cc). “n cases” refers to
the number of cases with the relevant cc values.

| k | all cases | cc = 0 | cc³1 | cc³5 |
|---|---|---|---|---|
| 1 | 0.000 | 0.000 | 0.000 | 0.000 |
| 2 | 0.000 | 0.000 | 0.000 | 0.000 |
| 3 | 0.004 | 0.000 | 0.004 | 0.009 |
| 5 | 0.027 | 0.000 | 0.030 | 0.036 |
| 10 | 0.140 | 0.000 | 0.156 | 0.205 |
| 15 | 0.315 | 0.000 | 0.351 | 0.446 |
| 20 | 0.482 | 0.000 | 0.537 | 0.616 |
| 30 | 0.708 | 0.000 | 0.788 | 0.795 |
| 40 | 0.837 | 0.192 | 0.909 | 0.929 |
| 50 | 0.899 | 0.462 | 0.948 | 0.973 |
| n cases | 257 | 26 | 231 | 112 |

Multiscore Performance is not Impacted when Multiple Diseases are Associated with a Gene

Multiscore performance were compared on genes with a single disease association (n=8,183, 73.4% cases) to those with multiple disease associations (n=2,972, 26.6% cases) (Table 21). Linear regression analysis suggested there is a statistically significant negative relationship between number of diseases and rank (not shown), however qualitatively, the distribution of ranks of these two classes of gene were similar.

TABLE 21
Average recall at k of Multiscore for all cases based on
GDx disease count of the positive gene. “n cases”
refers to the number of cases where the positive gene has
the relevant number of diseases in the GDx knowledgebase.

| | | 2+ |
|---|---|---|
| k | 1 disease | diseases |
| 1 | 0.306 | 0.401 |
| 2 | 0.451 | 0.556 |
| 3 | 0.540 | 0.657 |
| 5 | 0.664 | 0.766 |
| 10 | 0.821 | 0.874 |
| 15 | 0.890 | 0.922 |
| 20 | 0.927 | 0.951 |
| n cases | 7,311 | 2,677 |

### Multiscore Outperforms Other Phenotype-Only Prioritization Tools

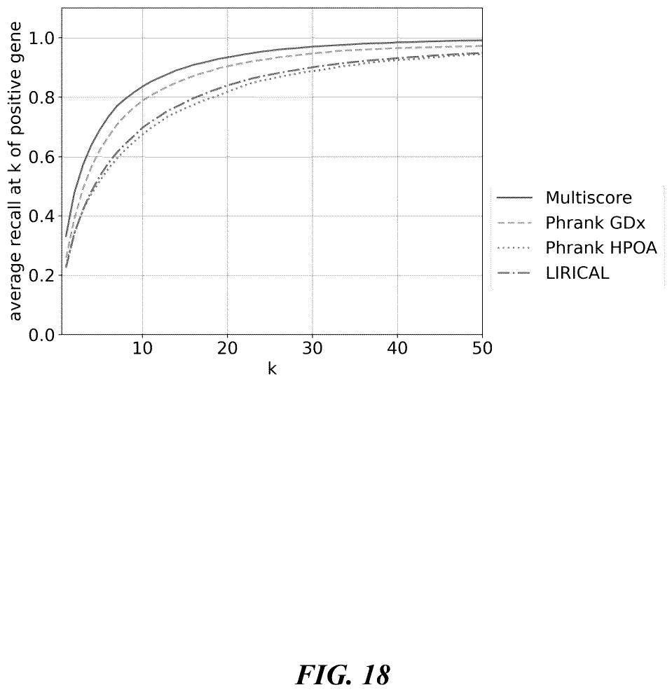

Multiscore showed higher average recall compared to Phrank GDx, Phrank HPOA, and LIRICAL (Table 16 and FIG. 18). At each k value, the best next performer was Phrank GDx; Phrank HPOA and LIRICAL average recall scores were lower and within 4% of each other for all tested k values.

TABLE 16
Ranking performance of Multiscore, Phrank with GDx GPA
knowledgebase (Phrank GDx), Phrank with HPOA GPA knowledgebase
(Phrank HPOA), and LIRICAL. Average recall at k, median
rank, mean rank, and rank standard deviation all refer
to prioritization of the positive reported gene.

| Average | | Phrank | Phrank | LIRICAL |
|---|---|---|---|---|
| recall at k | Multiscore | GDx | HPOA | HPOA |
| 1 | 0.331 | 0.258 | 0.231 | 0.225 |
| 2 | 0.479 | 0.392 | 0.342 | 0.341 |
| 3 | 0.572 | 0.492 | 0.419 | 0.420 |
| 5 | 0.691 | 0.623 | 0.519 | 0.535 |
| 10 | 0.835 | 0.788 | 0.673 | 0.696 |
| 15 | 0.898 | 0.861 | 0.762 | 0.782 |
| 20 | 0.933 | 0.904 | 0.818 | 0.839 |
| Median | 3 | 4 | 5 | 5 |
| rank | | | | |
| Mean rank | 6.35 | 10.8 | 15.4 | 14 |
| Rank | 10.24 | 35.64 | 38.7 | 35 |
| standard | | | | |
| deviation | | | | |

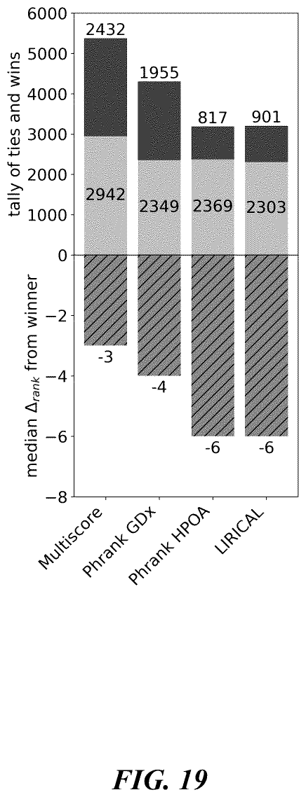

The overall wins tally of Multiscore and each algorithm is shown in the top bar chart of FIG. 19. Multiscore gave the highest ranking for the positive finding in 2,432 cases and tied for highest in an additional 2,942 cases, for a total of 5,374/9,989 (53.8%) cases tested, followed by Phrank GDx, which was the highest-ranking score in 1,955 cases and tied for highest in 2,349 cases, for a total of 4,304/9,989 (43.1%) cases. Multiscore showed the smallest median Dᵣₐₙₖ (see above) at −3.

A prioritization tool that uses a single GPA knowledgebase cannot prioritize a missing gene. In 217 cases without GDx GPA data and 257 cases without HPOA GPA data for the positive gene, Phrank GDx and Phrank HPOA provided no prioritization, respectively. LIRICAL includes ORPHANET disease entries which added phenotype annotations for the positive gene in an additional 30 cases.

### Multiscore can Handle Real World Patients

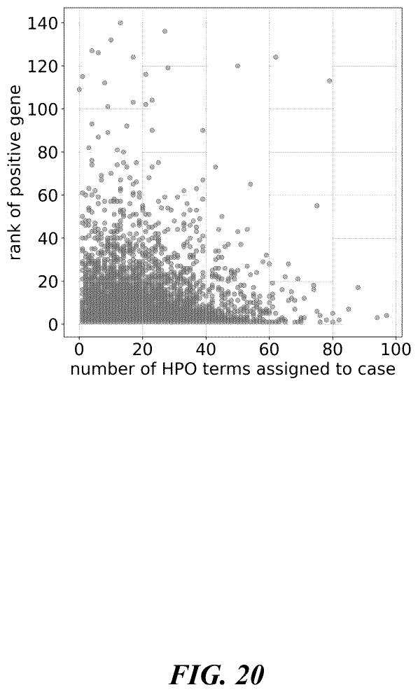

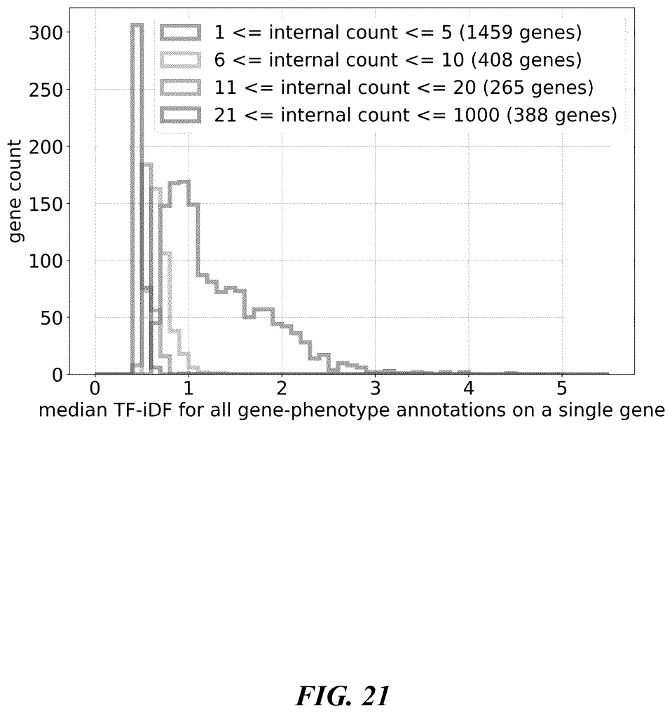

Clinical information for the knowledge reference and the testing set is abstracted from clinical notes of a diverse dataset (Table 18) without curation. The median number of HPO terms per case in the test set was 15; the performance of Multiscore was independent of the number of HPO terms (FIG. 20). As more cases are assigned to a given gene, the GPA set incorporates “random” phenotypes not directly related to the positive finding in a case. These phenotypes, although rare in the patients assigned to a gene, are common and nonspecific across other disease genes; in other words, those GPAs have a low TF-iDF for the given gene. FIG. 21 contains histogram data on the median TF-iDF GPA for genes in the GDx dataset.

### Discussion

Uncoupling genotype from phenotypic fit allows analysts to review molecularly strong findings that could represent phenotypic expansions or completely new disease associations; however, for known diseases, phenotype assessment is paramount. Multiscore combines curated datasets of gene-phenotype associations and real-world clinical data to rank the likelihood of a match between a gene and an individual's phenotype. Existing algorithms assess phenotypic similarity compared to a reference set of Mendelian conditions that need expert and often manual curation. Nonetheless, the phenotypes in individuals with a positive finding identified in clinical practice provide clues that can guide future molecular diagnoses, and it behooves us to leverage our internal knowledgebase.

The differences between Phrank GDx and Phrank HPOA illustrate the importance of the GPA knowledgebase and the fact that the GDx HRSS and Jaccard subscores overtake their Lit dataset counterparts at higher values of k also suggests that the GDx GPAs describe a more complete picture of the phenotypic spectrum of gene disease associations. Although future work is still needed to address phenotypic noise as our dynamic dataset continues to grow, Multiscore performance remained consistent despite incorporating low TF-iDF terms, since it combines both curated datasets and real-world data to create the knowledge reference. Combined with Literature Surveyor, the GDx clinical dataset also allows us to rescue those genes not yet curated in OMIM and contributed to the diagnosis of an additional 2.3% of cases.

Multiscore's ensemble architecture facilitates improvement by adding subscores from new algorithms like Phrank or new knowledgebases like the patient-level data from Monarch Initiative's Phenopacket Store. Newer transformer-based language model embeddings could be explored, such as ClinicalBERT, BioBERT, and PubMedBERT, to potentially capture nuanced gene-phenotype representations that may reside within their contextual frameworks. Including “negative phenotypes” from tests like MRIs and echocardiograms was considered. Finally, understanding the spectrum of ultra-rare disorders will continue to be a challenge that perhaps can be overcome using knowledge graphs and other architectures that explore disease space through protein-protein interactions, molecular functions, and biological networks, as in SHEPHERD, Phenolyzer, and phen2gene.

In this sense, Multiscore will contribute to scalability in case throughput, broader access and more diagnoses for patients.

Supplementary Methods I/O: Multiscore returns the following outputs:
Probability score for each gene
Eight subscore values for each gene
Internal case count for each gene

The range of possible subscore values is [0.0,1.0] with 0.0 representing no match and 1.0 representing a perfect match. The internal case count is defined as the number of Disease Phenotype Resource (DiPR, see below) single-diagnosis positive (SDP) cases found for each gene in the date-versioned DiPR case table that was used to build the DiPR-based gene-phenotype annotations. The rank is the fractional rank of the gene against all tested genes. The range for this value is (0.0, 1.0] where higher numbers indicate higher rank. Genes with equal probability scores get the highest possible ranks.

The RF model has been trained on retrospective case data to learn the relationship between the diagnostic/nondiagnostic disease state of a gene in a patient case (the labels) and the eight subscores (the features). The model prediction is therefore an inference on which genes could lead to a diagnostic finding in a patient case. The classifier is an RF model, an ensemble averaging of many decision trees trained on subsets of the data. Multiscore uses the scikit-learn implementation of RF, specifically sklearn.ensemble.RandomForestClassifier in scikit-learn version 1.1.2 (seikit-learn.org/stable/index.html). During training, the negative labels were randomly downsampled to a negative: positive ratio of 5:1. The classifier is trained on a time-window of SDP cases from the DiPR dataset; see below for details.

### Hybrid Relative Semantic Similarity (HRSS)

HRSS does not require exact phenotype matches to return a nonzero similarity score. If two phenotypes are on the same main branch of the HPO tree, the HRSS similarity of these two terms will be greater than zero. The “main branches” or “chapters” of the HPO tree are represented by the terms that are the child terms of HP:0000118 “Phenotypic Abnormality” which is the root term of the relevant subontology of the HPO: hpo.jax.org/app/browse/term/HP:0000118. Individual HRSS scores are defined between pairs of HPO terms. The HRSS subscore used in Multiscore is the best match average (BMA) of pairwise HRSS scores two lists of HPO terms (the case phenotype list and the gene phenotype list):
1. For two lists of HPO terms of length m and n, generate a matrix of size m*n of the pairwise HRSS scores.
2. Generate a best match vector of size m+n, comprising the top score in each of m rows and n columns of the pairwise matrix.
3. Calculate the BMA via the arithmetic mean of the best match vector.

HRSS in Multiscore is implemented using the Phenopy method phenopy.score.Scorer.score_hpo_pair_hrss. For HRSS HPOA, the most relevant disease per gene is used as the final score: each disease in the OMIM knowledgebase is scored, and for genes with more than one disease, the highest score is assigned as the HRSS HPOA subscore for that gene.

### Jaccard

Jaccard similarity considers the number of exact matches of members of two lists of HPO terms; it is defined as the length of the intersection of two lists divided by the length of the union of two lists. The Jaccard subscore between a case and a gene is defined by the case terms and gene terms in the gene-phenotype annotation file. Jaccard similarity in Multiscore is implemented using the Phenopy method phenopy.score.Scorer.score.

### HPO-Based Word2Vec Model

word2vec is a “bag-of-words” NLP method that measures a quantitative contextual similarity between two words using word embedding, the process of converting a word into a vector of numbers (“word to vector”). The vector can then be compared to other vector-embedded words using standard algorithms like cosine similarity. A word2vec model learns the context of words in a vocabulary by training a neural net. The context of a word is defined as the words that typically appear near it in a document.

In Multiscore a pretrained word2vec model was used. The vocabulary for this model is the phenotype terms in the HPO and was trained in-house to learn the context of phenotypes from analyzing the relevant literature. HPO terms were identified using the software tool txt2hpo (github.com/GeneDx/txt2hpo). A vocabulary of 6,630 HPO terms was learned by the word2vec model. Each HPO term is represented by a vector with a length of 400.

Related words tend to have similar contexts, therefore, the embeddings of related words tend to be “close” to each other by cosine similarity, and cosine similarity serves as a quantitative relationship between two words in the trained vocabulary. See Table 22 for the cosine similarity between two related words, “HP:0001250” (“Seizure”) and “HP:0007359” (“Focal-onset seizure”), and much lower cosine similarity between two unrelated words, “HP:0001250” (“Seizure”) and “HP:0010442” (“Polydactyly”).

TABLE 22
Cosine similarity of word embeddings (vectors)
between related and unrelated words in the
HPO word2vec model committed to phenopy.

| HPO | HPO term | HPO | HPO term | Cosine |
|---|---|---|---|---|
| term 1 | 1 name | term 2 | 2 name | similarity |
| HP: 0001250 | “Seizure” | HP: 0007359 | “Focal-onset | 0.505 |
| | | | seizure” | |
| HP: 0001250 | “Seizure” | HP: 0010442 | “Polydactyly” | 0.118 |

In Multiscore, the word2vec similarity is implemented in phenopy. The word2vec similarity between a list of case phenotypes and a list of GPAs for a single gene is defined as the cosine similarity between the average vector embeddings for each of the two lists.

doc2vec

doc2vec is a neural network-based NLP algorithm for semantic similarity measurements. As the name implies, doc2vec creates document embeddings, where an entire document is converted into one vector (“document to vector”). All documents in the Literature Surveyor corpus are converted into vectors of the same size which allows documents to be compared regardless of their original length aka word count. In our implementation of the Literature Surveyor doc2vec model in Multiscore, a document is defined in two ways: 1) a single sentence from gene-linked publications in the Literature Surveyor corpus, 2) the medical text of a single patient case.

The doc2vec model is trained in-house to learn the embeddings of medical text describing patients diagnosed with genetic disease. Multiscore creates a document embedding of case medical text via the doc2vec model, then this embedded vector is compared to the embeddings of documents (sentences) in the corpus linked to the genes of interest. These document-gene relationships are defined by the gene2pubmed table from NCBI. For a given gene, the doc2vec subscore is the maximum cosine similarity value of all tested documents.

The case text is preprocessed by transforming multiword phenotypes into a single “word” aka token, e.g., “Global developmental delay” (“HP:0001263”) becomes “global_developmental_delay.” The case text is subsequently embedded using DOC2VEC_MODEL.infer_vector. The document embedding vector length for case text and surveyor corpus sentences is 200.

All relevant genes in the case gene list are scored simultaneously. The gene2pubmed data table contains HGNC IDs linked to document keys from Literature surveyor and is filtered down to contain only documents in the corpus that match the relevant genes. The word embeddings from these relevant documents are loaded into the object category_docvecs and are scored against the case medical text document embeddings using cosine similarity applied by category_docvecs.most_similar. The result is a table that contains the highest cosine similarity score for each gene aka the doc2vec subscores for each gene.

### More Data Dependencies

Other data dependencies for Multiscore include:
[1] The HPO graph. For example, the Sep. 1, 2023 release of the HPO: github.com/obophenotype/human-phenotype-ontology/releases/download/v2023-09-01/hp.obo
[2] The HPOA OMIM disease-phenotype annotations. For example, the Sep. 1, 2023 release of the HPOA: github.com/obophenotype/human-phenotype-ontology/releases/download/v2023-09-01/phenotype.hpoa
[3] xa_phen_filter.pkl: table of multi-word (complex) phenotype names in the HPO network. In Natural Language Processing (NLP) these are called “multi-word units” or “collocations”. During text encoding (word2vec and doc2vec scoring) multi-word units in the HPO network were considered, for example, the words “generalized”, “abnormality”, “of” and “skin” do not have significant meaning separately, however “generalized abnormality of skin” is the name of “HP:0011354”. Therefore, by scanning text for complex phenotype names and replacing whitespace with underscores (“generalized abnormality of skin” is converted to “generalized_abnormality_of_skin”) before encoding, a single token was created for a multi-word unit. This table is built by Literature surveyor.
[4] Mim2gene table: key of OMIM disease ID values to gene symbols. Used to flatten disease-level information to gene-level information. This file is provided by OMIM.
[5] Gene2pubmed: regularly updated database containing manually curated associations between NCBI Gene and PubMed literature.

### Phrank Implementation in GORPipe

Phrank was implemented in our GORdb query system, leveraging built-in commands for handling direct-acyclic-graph (DAGMAP) and parallelization. Our implementation was tested against the published algorithm and the source code and demo data provided for Phrank (github.com/meng-ma-biomedical-AI/F29_Phrank/blob/master/demo/demo_phrank.py) and found to give the same results.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A method comprising:
(a) extracting one or more nucleic acid fragments of the individual obtained or derived from a biological sample of the individual;
(b) isolating one or more exomes of the one or more nucleic acid fragments; and
(c) performing genomic sequencing of the extracted one or more nucleic acid fragments, or performing exome sequencing of the isolated one or more exomes, or both, of one or more genes.

2. The method of claim 1, wherein isolating the one or more exomes of the one or more nucleic acid fragments comprises isolating a portion of the extracted one or more nucleotide fragments.

3. The method of claim 1, wherein the genomic sequencing comprises clinical-site genome sequencing, clinical-site exome sequencing, genome sequencing remotely from the clinical site, exome sequencing remotely from the clinical site, genome sequencing at a sequencing provider facility, exome sequencing at a sequencing provider facility, or any combination thereof.
